(19) United States
(12) Patent Application Publication
(10) Pub. No.: US 2018/0256699 A1
(43) Pub. Date: Sep. 13, 2018

(54) BORDETELLA ADENYLATE CYCLASE TOXIN VACCINES AND NEUTRALIZING ANTIBODIES

(71) Applicant: BOARD OF REGENTS, THE UNIVERSITY OF TEXAS SYSTEM, Austin, TX (US)

(72) Inventors: Xianzhe WANG, Austin, TX (US); Jennifer A. MAYNARD, Austin, TX (US)

(21) Appl. No.: 15/533,766

(22) PCT Filed: Dec. 8, 2015
(86) PCT No.: PCT/US15/64547

§ 371 Date: Jun. 7, 2017

Related U.S. Application Data
(60) Provisional application No. 62/089,000, filed on Dec. 8, 2014.

(51) Int. Cl. A61K 39/02; C07K 16/12; C07K 16/40; C12N 9/88; A61K 39/00

(52) U.S. Cl. CPC A61K 39/099; C07K 16/1225; C07K 16/40; C07K 2317/34; C12N 9/88; A61K 2039/575; C07K 2317/76; C12Y 406/01001

(57) ABSTRACT

The present disclosure describes immunogenic portions of Bordetella adenylate cyclase toxin (ACT), and neutralizing antibodies specific for such polypeptides. The antibodies can be used for diagnosis and anti-Bordetella therapies. Further provided are vaccine compositions including recombinant Bordetella adenylate cyclase toxin polypeptides.

### CROSS-REFERENCES TO RELATED APPLICATIONS

This application claims the benefit of U.S. Provisional Patent Application No. 62/089,000, filed Dec. 8, 2014, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under GM095638 awarded by the National Institutes of Health. The Government has certain rights in the invention. This work was further supported by a grant from the Welch Foundation (Grant No. F-1767).

### REFERENCE TO A “SEQUENCE LISTING,” A TABLE, OR A COMPUTER PROGRAM LISTING APPENDIX SUBMITTED ON A COMPACT DISK

The Sequence Listing written in file 48932-524001WO_ST25.TXT, created on Dec. 8, 2015, 129,614 bytes, machine format IBM-PC, MS-Windows operating system, is hereby incorporated by reference in its entirety for all purposes.

### BACKGROUND OF THE INVENTION

Whooping cough is a highly infectious disease caused primarily by the bacteria Bordetella pertussis. While incidence has dropped dramatically since the initiation of widespread vaccination programs using killed bacteria in the 1940s, in recent years rates have rebounded dramatically, reaching a 60-year high in the US in 2012 (1-3). This trend is especially troubling for unimmunized infants, who are most susceptible to the disease and exhibit the highest rates of morbidity and mortality. Modified vaccination strategies, including booster immunization of adolescents, adults and pregnant women, have been implemented to reduce transmission to neonates.

This increase in disease incidence coincides with the switch from whole cell to acellular vaccines in the 1990s, and has been attributed to several factors, including increased awareness, mismatch between vaccine and circulating strains, a Th1/Th2 immune response instead of the more effective Th1 response and a shorter duration of protection conferred by acellular vaccines (4). Recently, Warfel et al. demonstrated that acellular vaccines protect against disease symptoms but not subclinical infection and transmission in a novel non-human primate model (5). Taken together, these data provide a compelling argument for modification of the current vaccine.

Currently licensed acellular vaccines contain chemically detoxified pertussis toxin (PTx) and up to four surface adhesins, including filamentous hemagglutinin, pertactin and fimbriae 2/3. Exciting approaches in development to enhance vaccine-mediated protective immunity include a genetically attenuated B. pertussis for intranasal delivery (6); nanoparticle formulations including purified antigens and novel adjuvant formulations (7); as well as inclusion of additional highly conserved antigens in the current vaccine (8,9). A strong candidate for inclusion in any of these is the adenylate cyclase toxin (ACT) which aids in immune evasion and is produced by three closely related Bordetella species, including B. pertussis, B. parapertussis, and B. bronchiseptica (10,11).

ACT-deficient Bordetella strains have shown significantly compromised colonization and persistence in various mouse models (12-14), while some hypervirulent strains express higher ACT levels (15). Moreover, active or passive immunization with polyclonal anti-ACT antibodies protected mice against lethal respiratory challenges by B. pertussis and B. parapertussis (15) and shortened the period of bacterial colonization in the respiratory tract (16). Finally, natural infection of humans results in a strong anti-ACT antibody response (17).

ACT is a large, ˜177 kDa protein, consisting of two functionally discrete regions: the catalytic domain (residues 1-385), and a pore-forming or hemolysin region which is part of the larger repeat in toxin (RTX) family, represented in >250 bacterial strains (FIG. 1A). After translocation into the cytosol, the catalytic domain binds eukaryotic calmodulin with low nanomolar affinity (18) and rapidly converts available ATP to cAMP via its adenylate cyclase activity (19). The resulting supraphysiological cAMP levels disrupt signaling and bactericidal activities in phagocytic cells (20-22). The C-terminal 1300 residues exhibit homology to the E. coli α-hemolysin. This region consists of a hydrophobic domain capable of forming a cation-selective transmembrane channel (residues 525-715) (23), a modification region bearing two acylation sites at residues Lys860 and Lys983 (24), the RTX domain (residues 1006-1600), consisting of ˜40 calcium binding sites formed by glycine- and aspartate-rich nonapeptide repeats and finally, a C-terminal secretion signal (1600-1706). The RTX region also harbors the receptor-binding site, with specificity for the α_{M}β₂ integrin (also called CR3, Mac-1 and CD11b/CD18) present on phagocytic leukocytes (25,26). Both post-translational acylation by the co-expressed enzyme CyaC and calcium ion-mediated structural changes are essential for receptor binding, cAMP intoxication and pore-forming activities (24,27).

Despite evidence indicating ACT is a protective antigen, few neutralizing antibodies have been described and the location of neutralizing epitopes remains unclear. Moreover, ACT is prone to aggregation and degradation when produced by Bordetella or recombinantly by E. coli, precluding its inclusion in current acellular vaccine formulations (28). Therefore, we aimed to identify neutralizing antibodies and their domain specificity and determine whether any single domain, possessing desirable expression and protein stability characteristics, can recapitulate the antibody responses induced by the holo-toxin.

### BRIEF SUMMARY OF THE INVENTION

The present disclosure provides antibodies specific for a single domain of ACT that recapitulate the antibody responses induced by the holo-toxin and provide a dominant neutralizing antibody response. Mice were immunized with ACT, and the resulting antibodies were screened for binding to purified ACT. The vast majority of unique antibodies identified bound the C-terminal repeat-in-toxin (RTX) domain. Representative antibodies binding two non-overlapping, neutralizing epitopes in the RTX domain prevent ACT association with J774A.1 macrophages and soluble α_{M}β₂ integrin, suggesting that these antibodies inhibit the ACT-receptor interaction. Sera from mice immunized with the RTX domain showed similar neutralizing activity as ACT-immunized mice, indicating that this domain induced an antibody response similar to that induced by ACT. These data show that RTX can elicit neutralizing antibodies, which can in turn be used to treat Bordetella infection.

Provided herein are antibodies that compete for binding to the RTX domain of Bordetella ACT with an antibody comprising sequences selected from the group consisting of: light chain CDR1, CDR2, and CDR3 sequences of SEQ ID NO:1 and heavy chain CDR1, CDR2, and CDR3 sequences of SEQ ID NO:2; light chain CDR1, CDR2, and CDR3 sequences of SEQ ID NO:3 and heavy chain CDR1, CDR2, and CDR3 sequences of SEQ ID NO:4; light chain CDR1, CDR2, and CDR3 sequences of SEQ ID NO:5 and heavy chain CDR1, CDR2, and CDR3 sequences of SEQ ID NO:6; light chain CDR1, CDR2, and CDR3 sequences of SEQ ID NO:7 and heavy chain CDR1, CDR2, and CDR3 sequences of SEQ ID NO:9; light chain CDR1, CDR2, and CDR3 sequences of SEQ ID NO:9 and heavy chain CDR1, CDR2, and CDR3 sequences of SEQ ID NO:10; and light chain CDR1, CDR2, and CDR3 sequences of SEQ ID NO:11 and heavy chain CDR1, CDR2, and CDR3 sequences of SEQ ID NO:12. In embodiments, the antibody comprises sequences selected from the group consisting of: light chain CDR1, CDR2, and CDR3 sequences of SEQ ID NO:1 and heavy chain CDR1, CDR2, and CDR3 sequences of SEQ ID NO:2; light chain CDR1, CDR2, and CDR3 sequences of SEQ ID NO:3 and heavy chain CDR1, CDR2, and CDR3 sequences of SEQ ID NO:4; light chain CDR1, CDR2, and CDR3 sequences of SEQ ID NO:5 and heavy chain CDR1, CDR2, and CDR3 sequences of SEQ ID NO:6; light chain CDR1, CDR2, and CDR3 sequences of SEQ ID NO:7 and heavy chain CDR1, CDR2, and CDR3 sequences of SEQ ID NO:9; light chain CDR1, CDR2, and CDR3 sequences of SEQ ID NO:9 and heavy chain CDR1, CDR2, and CDR3 sequences of SEQ ID NO:10; and light chain CDR1, CDR2, and CDR3 sequences of SEQ ID NO:11 and heavy chain CDR1, CDR2, and CDR3 sequences of SEQ ID NO:12. In embodiments, the antibody comprises sequences selected from the group consisting of: light chain variable region sequence of SEQ ID NO:1 and heavy chain variable region sequence of SEQ ID NO:2; light chain variable region sequence of SEQ ID NO:3 and heavy chain variable region sequence of SEQ ID NO:4; light chain variable region sequence of SEQ ID NO:5 and heavy chain variable region sequence of SEQ ID NO:6; light chain variable region sequence of SEQ ID NO:7 and heavy chain variable region sequence of SEQ ID NO:9; light chain variable region sequence of SEQ ID NO:9 and heavy chain variable region sequence of SEQ ID NO:10; and light chain variable region sequence of SEQ ID NO:11 and heavy chain variable region sequence of SEQ ID NO:12.

In embodiments, the antibody comprises light chain CDR1, CDR2, and CDR3 sequences of SEQ ID NO:1 and heavy chain CDR1, CDR2, and CDR3 sequences of SEQ ID NO:2. In embodiments, the antibody comprises light chain CDR1, CDR2, and CDR3 sequences of SEQ ID NO:3 and heavy chain CDR1, CDR2, and CDR3 sequences of SEQ ID NO:4. In embodiments, the antibody comprises light chain CDR1, CDR2, and CDR3 sequences of SEQ ID NO:5 and heavy chain CDR1, CDR2, and CDR3 sequences of SEQ ID NO:6. In embodiments, the antibody comprises light chain CDR1, CDR2, and CDR3 sequences of SEQ ID NO:7 and heavy chain CDR1, CDR2, and CDR3 sequences of SEQ ID NO:9. In embodiments, the antibody comprises light chain CDR1, CDR2, and CDR3 sequences of SEQ ID NO:9 and heavy chain CDR1, CDR2, and CDR3 sequences of SEQ ID NO:10. In embodiments, the antibody comprises light chain CDR1, CDR2, and CDR3 sequences of SEQ ID NO:11 and heavy chain CDR1, CDR2, and CDR3 sequences of SEQ ID NO:12.

Also provided herein are antibodies that compete for binding to the RTX domain of Bordetella ACT with an antibody comprising sequences selected from the group consisting of: light chain CDR1, CDR2, and CDR3 sequences of SEQ ID NO:13 and heavy chain CDR1, CDR2, and CDR3 sequences of SEQ ID NO:14; light chain CDR1, CDR2, and CDR3 sequences of SEQ ID NO:15 and heavy chain CDR1, CDR2, and CDR3 sequences of SEQ ID NO:16; and light chain CDR1, CDR2, and CDR3 sequences of SEQ ID NO:17 and heavy chain CDR1, CDR2, and CDR3 sequences of SEQ ID NO:18. In embodiments, the antibody comprises sequences selected from the group consisting of CDR1, CDR2, and CDR3 sequences of SEQ ID NO:13 and heavy chain CDR1, CDR2, and CDR3 sequences of SEQ ID NO:14; light chain CDR1, CDR2, and CDR3 sequences of SEQ ID NO:15 and heavy chain CDR1, CDR2, and CDR3 sequences of SEQ ID NO:16; and light chain CDR1, CDR2, and CDR3 sequences of SEQ ID NO:17 and heavy chain CDR1, CDR2, and CDR3 sequences of SEQ ID NO:18. In embodiments, the antibody comprises sequences selected from the group consisting of light chain variable region sequence of SEQ ID NO:13 and heavy chain variable region sequence of SEQ ID NO:14; light chain variable region sequence of SEQ ID NO:15 and heavy chain variable region sequence of SEQ ID NO:16; and light chain variable region sequence of SEQ ID NO:17 and heavy chain variable region sequence of SEQ ID NO:18.

In embodiments, the antibody comprises light chain CDR1, CDR2, and CDR3 sequences of SEQ ID NO:13 and heavy chain CDR1, CDR2, and CDR3 sequences of SEQ ID NO:14. In embodiments, the antibody comprises light chain CDR1, CDR2, and CDR3 sequences of SEQ ID NO:15 and heavy chain CDR1, CDR2, and CDR3 sequences of SEQ ID NO:16. In embodiments, the antibody comprises light chain CDR1, CDR2, and CDR3 sequences of SEQ ID NO:17 and heavy chain CDR1, CDR2, and CDR3 sequences of SEQ ID NO:18.

In embodiments, the antibody binds the RTX domain of Bordetella ACT with a Kd of 0.01-100 nM, e.g., 0.1-100 nM, 0.5-50 nM, or 0.1-20 nM. In embodiments, the antibody is humanized, e.g., with 1, 2, 3, 4, 5, 6, 7, or 8 framework regions derived from a human antibody. In embodiments, the antibody further comprises a constant region, e.g., derived from a human antibody.

In embodiments, the antibody is a single chain antibody. In embodiments, the antibody is an Fab, Fab′, or F(ab′)₂. In embodiments, the antibody is a humanized single chain antibody. In embodiments, the antibody is a humanized Fab, Fab′, or F(ab′)₂. In embodiments, the single chain antibody comprises a light chain variable region (optionally humanized), a linker consisting of 4-100 amino acids, and a heavy chain variable region (optionally humanized). In embodiments, the linker is 4-40 amino acids, e.g., 10-30, or 16-24 amino acids.

In embodiments, the antibody is attached to a detectable label, e.g., a fluorescent label or affinity tag. In embodiments, the antibody is attached to a stabilizing agent, e.g., PEG or PEG derivative.

Provided herein are methods for diagnosing a Bordetella infection in an individual comprising contacting a biological sample from the individual with an antibody as described above; determining if the antibody binds a component of the biological sample (i.e., if the antibody binds Bordetella ACT in the biological sample); and diagnosing Bordetella infection in the individual when the antibody binds a component of the biological sample. In embodiments, the biological sample is saliva, mucus, sputum, blood (or serum), taken from a nasal pharangeal swab or biopsy, a cellular sample, or cell lysate. In embodiments, the determining is carried out using an ELISA (e.g., a sandwich ELISA using antibodies specific for different epitopes of ACT).

Further provided are pharmaceutical compositions comprising an antibody as described above and a pharmaceutically acceptable excipient. In embodiments, the pharmaceutical composition comprises more than one antibody as described above, e.g., antibodies that bind different epitopes of the RTX domain. In embodiments, the pharmaceutical composition further comprises an antibacterial agent.

Provided are methods for treating or preventing Bordetella infection in an individual comprising administering a pharmaceutical composition as described above to the individual, thereby treating or preventing Bordetella infection in an individual. In embodiments, the administering is to a mucosal surface, e.g., in a nasal or oral aerosol. In embodiments, the administering is parenteral, e.g., intraperitoneal, intravenous, or subcutaneous. In embodiments, the individual is a human. In embodiments, the pharmaceutical composition is administered more than once. In embodiments, the pharmaceutical composition is administered within about two weeks of exposure to Bordetella, e.g., within one week, three days, or one day from exposure. In embodiments, the pharmaceutical composition is administered with an additional therapeutic agent, e.g., an antibacterial agent, either simultaneously or separately.

In one aspect a vaccine composition is provided. The composition includes a recombinant Bordetella adenylate cyclase toxin polypeptide consisting essentially of a sequence corresponding to amino acid residues 700-1706 of Bordetella adenylate cyclase toxin protein or an immunogenic fragment thereof and a pharmaceutically acceptable vaccine excipient.

In one aspect a method for immunizing a subject in need thereof against Bordetella pertussis is provided. The method includes administering to the subject an effective amount of the vaccine as provided herein including embodiments thereof.

In another aspect, a method of preventing or treating whooping cough in a subject in need thereof is provided. The method includes administering to the subject a therapeutically effective amount of the vaccine as provided herein including embodiments thereof.

### BRIEF DESCRIPTION OF THE DRAWINGS

FIG. 1A-1C. Expression and purification of intact ACT and domains. FIG. 1 A, Adenylate cyclase toxin domain architecture. ACT is a 177 kDa protein toxin, consisting of four sequential domains: the catalytically active N-terminal adenylate cyclase (CAT) domain, the central hydrophobic (HP) domain, modification (Mod) region carrying two acylation sites K860 and K983, the C-terminal repeat-in-toxin (RTX) domain and finally a C-terminal secretion signal (SEC). The five shaded blocks in the RTX region represent tandem Gly-Asp repeats. FIG. 1 B, ACT forms high molecular weight species (>600 kDa) when urea is removed by dialysis or dilution. Size exclusion chromatograms of ˜120 ug ACT using a Superdex 200 column were collected directly after a 1:10 dilution (final urea concentration 0.8 M) or overnight dialysis into HBSC with 1 M urea. Arrow indicates the size expected for monomer, 177 kDa. FIG. 1 C, SDS-PAGE gel comparing full-length ACT with different domain constructs after purification from E. coli. Three versions of the CAT domain (residues 1-373 [CAT₃₇₃], 1-385 [CAT₃₈₅] and 1-400 [CAT₄₀₀]) and three versions of the RTX domain (residues 985-1706 [RTX₉₈₅], 751-1706 [RTX₇₅₁] and acylated 751-1706 [RTX₇₅₁]) were expressed from plasmid pET28a with an N-terminal His-tag. The HP domains (residues 399-1096 [HP₁₀₉₆] and acylated 399-1096 [HP₁₀₉₆]) were expressed from plasmid pMalc-5× with an N-terminal maltose binding protein (MBP) fusion for enhanced solubility and C-terminal His-tag.

FIG. 2A-2D. ACT domain oligomeric state and secondary structure. Purified domains were separated by size exclusion chromatography (Superdex200 column, except Superdex75 for CAT₄₀₀), with far UV circular dichroism spectra (Jasco J-815) used to assess secondary structure in the presence of 2 mM CaCl₂ or absence of calcium ions (10 mM EGTA). FIG. 2A, The catalytic domain, spanning residues 1-400 (CAT₄₀₀) eluted as a single peak of expected size. The secondary structure is similar to that observed in the CAT₃₇₃ crystal structure (56% helix, 14% strands, 13% turns and 17% unordered). FIG. 2B, The HP domain, spanning residues 399-1096, with an N-terminal MBP fusion protein eluted off SEC as high molecular weight aggregates whether acylated (*) or non-acylated. FIG. 2C, The RTX₉₈₅ formed high molecular weight aggregates in the absence of calcium ions, but eluted as two peaks, one corresponding to the expected molecular weight, 78 kDa in the presence of calcium. Circular dichroism revealed significant conformational change upon addition of 2 mM CaCl₂ corresponding to an increase in beta content. FIG. 2D, The RTX₇₅₁ domain exhibits a similar calcium-dependent delay in elution volume, which is more pronounced when the protein is acylated (*) and under these conditions yields a single monomer peak. Inset SDS-PAGE gels show proteins present in indicted peaks, with arrows indicating the expected monomer size.

FIG. 3A-3B. ACT and RTX domains bind purified α_{M}β₂ receptor. Soluble murine α_{M}β₂ receptor was coated onto ELISA plates, blocked and ACT or domains serially diluted in M-PBST. Bound protein was detected with polyclonal rabbit anti-ACT antibody and goat-anti-rabbit-HRP. To assess non-specific binding, control wells were not coated with α_{M}β₂ receptor but blocked with M-PBST only. (FIG. 3A) RTX₇₅₁* and (FIG. 3B) acylated ACT* showed receptor-dependent binding, although ACT also exhibited significant non-specific binding. All other domains showed no specific or non-specific binding.

FIG. 4A-4D. ACT immunization induces a diverse antibody response. FIG. 4A, A phylogenetic tree depicting antibody sequence relatedness was generated using the light and heavy variable region amino acid sequences. Neutralizing scAbs are colored gray, with unique shapes denoting recognition of distinct epitopes among this antibody group as determined by competition ELISA. Hollow shapes denote antibodies whose binding does not depend on the presence of calcium. Antibodies competing with previously characterized monoclonal antibodies are indicated; all antibodies bind RTX except M1C5, M1F11 and M2G5 which bind CAT₄₀₀. FIG. 4B, Representative SDS-PAGE of scAbs after purification by IMAC and Superdex S200. Arrow indicates expected size of ˜48 kDa, 2 μg each of M1F11, M1C12, M1H5, and M2B10 scAbs were loaded. FIG. 4C, Twenty-one unique scAbs identified from the immune phage libraries were tested for ACT neutralization of cAMP intoxication. ACT was incubated with scAb at a 160-fold molar excess before adding to J774A.1 cells. The resulting cAMP concentrations in the cellular lysate was determined by cAMP ELISA, with the total cAMP concentration divided by the protein concentration in lysate, and normalized to control cells treated with ACT only (empty bar). Error bars indicate range of duplicate assays. FIG. 4D, The 21 scAbs were evaluated for their ability to rescue J774A.1 macrophages from ACT-induced lysis. The protocol was similar to cAMP neutralization, used a 160-fold molar excess of antibody and a longer incubation time (2 hours). Cell lysis was measured via lactate dehydrogenase release using the Cytotox 96 kit (Promega).

FIG. 5A-5D. Two novel, neutralizing epitopes are present in the RTX domain. FIG. 5A, Two representative neutralizing scAbs were converted to chimeric IgG1/κ antibodies, in which the murine variable regions are appended by human constant domains. These were transiently expressed in CHO-K1 cells and purified by (NH₄)₂SO₄ precipitation and protein A affinity chromatography. SDS-PAGE (4-20% gradient gel, 2 μg loaded) shows high purity and expected size of the recombinant IgG: 25 kDa (light chain) and 50 kDa (heavy chain) when reduced, 150 kDa under non-reducing condition. ELISA demonstrates the ACT domain specificity of the FIG. 5B, M2B10 and FIG. 5C, M1H5 IgG antibodies. Microtiter plates were coated with ACT or ACT domains at equimolar concentrations, followed by serial dilution of antibody from 1 nM followed by detection with anti-human Fc antibody-HRP conjugate. FIG. 5D, Competition ELISA determined that the M2B10 and M1H5 antibodies bind novel, non-overlapping epitopes. A 200-fold molar excess (20 nM) of previously described murine mAbs (3D1, 2A12, 10A1, 2B12, 9D4, 6E1, 7C7, 1H6) (47) or scAb (M1H5, M2B10) proteins were mixed with M2B10 and M1H5 IgG (0.1 nM) and incubated on ACT-coated ELISA plate, with bound M2B10 or M1H5 detected as above. The absorbance is normalized to that of M2B10 or M1H5 with no competitor. Absorbance significantly <1.0 indicates competition between the antibody pair.

FIG. 6A-6D. M2B10 and M1H5 antibodies block ACT—α_{M}β₂ integrin interaction. FIG. 6A, Antibody neutralization of cAMP intoxication using J774A.1 presenting the α_{M}β₁ receptor and CHO-K1 cells lacking the receptor. Both assays were performed with a 160-fold molar excess of scAb over ACT. FIG. 6B, Antibody blockade of ACT binding to J774A.1 cells assessed by FACS. Biotinylated ACT was incubated with a 120-fold molar excess of scAb and added to 4×10⁵ J774A.1 suspension cells on ice. After washing, bound biotinylated ACT (B-ACT) was detected with streptavidin-PE and analyzed by FACS (mean fluorescence noted next to each peak). Controls include untreated cells (“Cells only”) and cells treated with non-biotinylated ACT followed by streptavidin-PE (“ACT”). FIG. 6C, Antibody blockade of ACT binding to soluble α_{M}β₂ integrin by ELISA. ACT (0.5 μg/mL) was incubated with serial dilutions of M2B10, M1H5, 3D1 and 2A12 antibodies at 5-, 1.6- and 0.5-fold molar excess, before transfer to an ELISA plate coated with murine α_{M}β₂ integrin. Bound ACT was detected with rabbit anti-ACT polyclonal antibody followed by HRP-conjugated goat anti-rabbit IgG antibody. FIG. 6D, Antibody neutralization of ACT secreted by B. pertussis. Antibodies at 5-, 1.6-, 0.5- and 0-fold molar ratios were incubated with live B. pertussis before adding to adherent J774A.1 cells. The resulting intracellular cAMP concentrations were measured, normalized to total protein concentration, and expressed as % intoxication.

FIG. 7A-7C. The RTX domain is immunodominant and elicits neutralizing antibodies. FIG. 7A, Immunization with ACT yields sera preferentially recognizing the RTX domain. Purified domains were coated at equal moles on microtiter plates, with sera serially diluted starting at 1:200. The average EC₅₀ for individual domains is shown. Error bars are the standard deviations of the EC₅₀ among four mice. FIG. 7B, Immunogenicity of purified domains. Mice were immunized with intact ACT or individual domains, with the serum EC₅₀ for ACT measured by ELISA after the first boost (6 weeks) or second boost (8 weeks). FIG. 7C, Sera from mice immunized with ACT and RTX₉₈₅ domain neutralize cAMP intoxication similarly. Sera from each immunization group at a 1:400 dilution were incubated with 125 ng/mL ACT in DMEM before adding to J774A.1 cells. Intracellular cAMP levels were measured by cAMP ELISA, divided by the total protein concentrations, and normalized to cells treated with ACT alone. “Pre” indicates baseline sera collected prior to immunization. Statistical significance was determined by one-way ANOVA with Tukey's test; ** indicates p≤0.01, *** p≤0.001. For all panels, an * indicates an acylated domain.

FIG. 8A-8B. Mice produce antibodies binding the M1H5 and M2B10 neutralizing epitopes whether immunized with (FIG. 8A) ACT or (FIG. 8B) RTX₉₈₅. Sera at a 1:200 dilution were incubated on ELISA plates coated with ACT, before addition of 0.1 nM of M2B10, M1H5, or M1F11 as a competitor. After incubation, immobilized monoclonal antibody was detected with anti-human Fc-HRP, with absorbance normalized to wells without sera. Lower absorbance indicates a higher concentration of epitope-specific murine antibodies.

FIG. 9. RTX dominates the human immune response to ACT. Nine serum samples from humans exposed to pertussis were tested for reactivity to the catalytic domain, RTX₇₅₁, or intact ACT by ELISA. Absorbance values at a 100-fold dilution of the sera was normalized to that of ACT at the same dilution. A paired t-test was used to determine the statistical significance between signals for CAT and RTX domains binding.

FIG. 10A-10B. Neutralizing antibodies specific for the RTX domain identified from antibody phage library. The antibodies were obtained from mice immunized with ACT or RTX. FIG. 10A, Similarity of RTX-specific antibody variable region sequences specific. Antibodies M1H5, M2C2, M1F10, M1E12, M2F9, and M2B5 bind to one epitope region of RTX, while M1E2, M1H1, and M2B10 bind to a distinct non-overlapping epitope region on RTX. FIG. 10B, Variable region sequences of antibodies specific for RTX. Residues are numbered according to Kabat numbering system, and CDRs assigned according to Kabat definitions (see the website available at bioinf.org.uk/abysis/sequence input/key annotation/key annotation.html).

M1H5 has light and heavy chain variable region sequences of SEQ ID NOs:1 and 2, respectively. M2C2 has light and heavy chain variable region sequences of SEQ ID NOs:3 and 4, respectively. M1F10 has light and heavy chain variable region sequences of SEQ ID NOs:5 and 6, respectively. M1E12 has light and heavy chain variable region sequences of SEQ ID NOs:7 and 8, respectively. M2F9 has light and heavy chain variable region sequences of SEQ ID NOs:9 and 10, respectively. M2B5 has light and heavy chain variable region sequences of SEQ ID NOs:11 and 12, respectively. M1E2 has light and heavy chain variable region sequences of SEQ ID NOs:13 and 14, respectively. M1H1 has light and heavy chain variable region sequences of SEQ ID NOs:15 and 16, respectively. M2B10 has light and heavy chain variable region sequences of SEQ ID NOs:17 and 18, respectively.

M1H5 binds RTX751 with an affinity of ˜1.6 nM, and inhibits ACT-receptor interaction with IC50=2.5 nM in J774. A cell neutralization (ACT=125 ng/mL). M2B10 binds RTX751 with an affinity of ˜1.2 nM, and inhibits ACT-receptor interaction with an IC50=6.0 nM. M2C2 binds RTX751 with an affinity of ˜1.5 nM, and inhibits ACT-receptor interaction with an IC50=8.1 nM. IC50 values were measured in J774A.1 macrophage cell line intoxication assay with 125 ng/mL ACT (0.7 nM).

FIG. 11A-11B. RTX fragments are equally immunogenic as intact ACT (FIG. 11A) and more immunogenic than the N-terminal catalytic and hydrophobic fragments (FIG. 11B).

FIG. 12. Mice immunized with ACT or RTX751 neutralize ACT in an in vitro toxin neutralization assay to a similar extent.

FIGS. 13A and 13B. Two receptor blocking antibodies bind distinct epitopes. FIG. 13A, ELISA to assess binding of the M1H5 (left panel) and M2B10 (right panel) antibodies to RTX from different Bordetella species. FIG. 13B, Intoxication assay to assess neutralization of ACT secreted by B. pertussis and B. Bronchi. In spite of similar mechanism of receptor blockade, two broadly neutralizing antibodies have distinct epitopes and antibody M2B10 recognizes ACT produced by all major Bordetella strains, while antibody M1H5 is specific for B. pertussis ACT. Left panel: B. pertussis; right panel: B. bronchioseptica.

FIG. 14. Epitopes of neutralizing antibodies: Predicted RTX751 structure model generated by RaptorX server. Dark gray residues implicated in M1H5 by mutagenesis studies; specific for B. pertussis ACT (major human pathogenic strain). Black: residues implicated in M2B10 antibody binding; conserved among all three major Bordetella strains. Arrow: presumably involved in structure/stability. Residues identified using a high throughput yeast display scheme; which was validated by purifying a number of variants with predicted residues followed by ELISA analysis.

FIG. 15. RTX751 structure model generated by RaptorX server. Dark gray: M1H5 epitope; black: M2B10 epitope.

### DETAILED DESCRIPTION OF THE INVENTION

### I. Introduction

The adenylate cyclase toxin (ACT) is a multifunctional virulence factor secreted by Bordetella species. Upon interaction of its C-terminal hemolysin moiety with the cell surface receptor α_{M}β₂ integrin, the N-terminal cyclase domain translocates into the host cell cytosol where it rapidly generates supra-physiological cAMP concentrations, which inhibit host cell anti-bacterial activities. Although ACT has been shown to induce protective immunity in mice, it is not included in any current acellular pertussis vaccines due to protein stability issues and poor understanding of its role as a protective antigen.

Provided herein are neutralizing antibodies that specifically bind to ACT, in particular the RTX (repeat in toxin) domain. Such antibodies can be used for passive immunotherapy and diagnosis of Bordetella infections. Further provided herein are highly protective vaccines including recombinant Bordetella adenylate cyclase toxin polypeptides corresponding to C-terminal fragments of ACT and uses thereof for the treatment and prevention of whooping cough.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “antibody” refers to a polypeptide comprising a framework region from an immunoglobulin gene, or fragments thereof, that specifically bind and recognize an antigen, e.g., β8, a particular cell surface marker, or any desired target. Typically, the “variable region” contains the antigen-binding region of the antibody (or its functional equivalent) and is most critical in specificity and affinity of binding. See Paul, Fundamental Immunology (2003). The term includes antibody fragments and antibody variants that specifically bind to the antigen.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

An “isotype” is a class of antibodies defined by the heavy chain constant region. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “V-region” refers to an antibody variable region domain comprising the segments of Framework 1, CDR1, Framework 2, CDR2, Framework 3, CDR3, and Framework 4. These segments are included in the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikani et al., J. Mol. Biol 1997, 273(4)). Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl Acad. Sci. USA, 86, 9268-9272 (1989); Martin, et al, Methods Enzymol., 203: 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” is an antibody molecule in which (a) the constant region, or a portion thereof, is altered, replaced or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody, polypeptide or fragment thereof) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds ACT will typically bind to ACT with at least a 2-fold greater affinity than a non-ACT target.

The term “binds” with respect to a particular target (e.g., ACT or α_{M}β₂ integrin) typically indicates that an antibody or recombinant Bordetella adenylate cyclase toxin polypeptide as provided binds a majority of the targets in a pure population of those targets. For example, an antibody that binds a given cell type typically binds to at least 2/3 of the cells in a population of the indicated cells (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

Similarly, a first recombinant polypeptide as provided herein “competes” for binding to a target (α_{M}β₂ integrin) with an antibody or a second recombinant polypeptide, when binding of the antibody or second recombinant polypeptide with the target is detectably decreased in the presence of the first recombinant polypeptide compared to the binding of the antibody or second polypeptide in the absence of the first recombinant polypeptide.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a polypeptide, cell or nucleic acid, or vector, indicates that the polypeptide, cell, nucleic acid or vector, has been modified by (i) the introduction of a heterologous polypeptide, nucleic acid or protein, or (ii) the alteration of a native polypeptide, nucleic acid or protein, or (iii) that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

The term “diagnosis” refers to a relative probability that a disorder such as infection or an inflammatory condition is present in the subject. Similarly, the term “prognosis” refers to a relative probability that a certain future outcome may occur in the subject. For example, prognosis can refer to the likelihood that an individual will develop a Bordetella infection, have recurrence, or the likely severity of the infection (e.g., severity of symptoms, rate of functional decline, survival, etc.). The terms are not intended to be absolute, as will be appreciated by any one of skill in the field of medical diagnostics.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, sputum, mucus, urine, feces, etc.

The terms “therapy,” “treatment,” and “amelioration” refer to any reduction in the severity of symptoms or duration of infection. As used herein, the terms “treat” and “prevent” are not intended to be absolute terms. Treatment and prevention can refer to any delay in onset of symptoms, amelioration of symptoms, reduced duration of infection or symptoms, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of infection or symptoms is reduced by at least 10%, as compared, e.g., to the individual before administration or to a control individual not undergoing treatment. In some aspects the severity of infection or symptoms is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the concentration of the antibody and/or associated components (e.g., excipients). The dose will vary depending on a number of factors, including frequency of administration; size and tolerance of the individual; severity of the infection; risk of side effects; and the route of administration. One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

### III. Antibodies

Provided herein are neutralizing antibodies that bind to Bordetella ACT, in particular the RTX domain. A neutralizing antibody can inhibit or block is the ability of the ACT protein to enter a cell, increase cAMP levels, and/or bind α_{M}β₂ integrin. In embodiments, the antibody binds with a Kd of less than about 100 nM, e.g., 0.01-100 nM, 0.1-20 nM, 0.5-10 nM. In embodiments, the antibody is humanized. The antibody can be of any isotype, e.g., IgG (IgG1, IgG2, IgG3, IgG4), IgM, IgA (IgA1, IgA2), IgD, and IgE isotype. In embodiments, the antibody is an antibody fragment (e.g., Fab, Fab′, F(ab)₂, scAb).

A. Antibody Fragments

Antibodies as provided herein may be Fab, Fab′, or F(ab)₂′ fragments. Where the antibodies are Fab, Fab′, or F(ab)₂′ fragments, the antibodies include a heavy chain (e.g. including a partial constant and a variable region) and a light chain (e.g. including a constant and a variable region). In embodiments, the antibody is a humanized Fab, Fab′, or F(ab)₂′ fragment.

In embodiments, the humanized antibody is a single chain antibody (scAb or scFv). A single chain antibody includes a variable light chain and a variable heavy chain. That is, a single chain antibody includes a single light chain and a single heavy chain, in contrast to an immunoglobulin (Ig) antibody, which includes two identical pairs of polypeptide chains, each pair having one light chain and one heavy chain. Each light chain and heavy chain (whether in an Ig or scAb) includes two regions: a variable (“V”) region (i.e. variable light chain and variable heavy chain) involved in binding the target antigen, and a constant (“C”) region that interacts with other components of the immune system. The variable light chain and the variable heavy chain in a single chain antibody can be linked through a linker peptide. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S, and Whitlow, M. (1988).

Methods of making scFv antibodies have been described. See, Huse et al., Science 246:1275-1281 (1989); Ward et al., Nature 341:544-546 (1989); and Vaughan et al., Nature Biotech. 14:309-314 (1996). For example, mRNA from B-cells from an immunized animal can be isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

B. Competitive Antibodies

In embodiments, the antibodies described herein compete with an antibody that is capable of binding Bordetella ACT, or a fragment thereof (e.g., comprising an immunogenic and soluble fragment of ACT, e.g., from the RTX domain). Where the antibody competes with an antibody (competitor antibody) for binding a target antigen, the antibody inhibits (completely or partially) binding of the competitor antibody to a measurable extent, e.g., binding of the competitor antibody to the antigen is reduced by at least 30%, usually at least about 40%, 50%, 60% or 75%, and often by at least about 90%, 95%, or more. Binding can be measured by any of the methods described herein.

In embodiments, the humanized antibody is an antibody which competes with an antibody that is capable of binding the RTX domain of Bordetella ACT. Thus, in embodiments, antibodies are provided that compete for binding to ACT with an antibody comprising sequences selected from the group consisting of: light chain CDR1, CDR2, and CDR3 sequences of SEQ ID NO:1 and heavy chain CDR1, CDR2, and CDR3 sequences of SEQ ID NO:2; light chain CDR1, CDR2, and CDR3 sequences of SEQ ID NO:3 and heavy chain CDR1, CDR2, and CDR3 sequences of SEQ ID NO:4; light chain CDR1, CDR2, and CDR3 sequences of SEQ ID NO:5 and heavy chain CDR1, CDR2, and CDR3 sequences of SEQ ID NO:6; light chain CDR1, CDR2, and CDR3 sequences of SEQ ID NO:7 and heavy chain CDR1, CDR2, and CDR3 sequences of SEQ ID NO:9; light chain CDR1, CDR2, and CDR3 sequences of SEQ ID NO:9 and heavy chain CDR1, CDR2, and CDR3 sequences of SEQ ID NO:10; and light chain CDR1, CDR2, and CDR3 sequences of SEQ ID NO:11 and heavy chain CDR1, CDR2, and CDR3 sequences of SEQ ID NO:12. In embodiments, antibodies are provided that compete for binding to ACT with an antibody comprising sequences selected from the group consisting of: light chain CDR1, CDR2, and CDR3 sequences of SEQ ID NO:13 and heavy chain CDR1, CDR2, and CDR3 sequences of SEQ ID NO:14; light chain CDR1, CDR2, and CDR3 sequences of SEQ ID NO:15 and heavy chain CDR1, CDR2, and CDR3 sequences of SEQ ID NO:16; light chain CDR1, CDR2, and CDR3 sequences of SEQ ID NO:17 and heavy chain CDR1, CDR2, and CDR3 sequences of SEQ ID NO:18. In embodiments, the antibody inhibits (completely or partially) ACT activity, e.g., by about 10%, 20%, 30%, 40%, 50%, 60%, 70%, 80%, 90% or 100%. In some further embodiments, the antibody does not compete for binding with an antibody selected from 3D1, 2A12, 10A1.

C. Humanized Antibodies

A humanized antibody is a genetically engineered antibody in which at least one CDR (or functional fragment thereof) from a mouse antibody (“donor antibody”, which can also be rat, hamster or other non-human species) are grafted onto a human antibody (“acceptor antibody”). In embodiments, more than one mouse CDR is grafted (e.g. all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and variable region framework (FR). The FR may form part of a constant region within a human antibody. In addition, in order to retain high binding affinity, amino acids in the human acceptor sequence may be replaced by the corresponding amino acids from the donor sequence, for example where: (1) the amino acid is in a CDR; (2) the amino acid is in the human framework region (e.g. the amino acid is immediately adjacent to one of the CDR's) (see, e.g., U.S. Pat. Nos. 5,530,101 and 5,585,089). Although humanized antibodies often incorporate all six CDRs (e.g. as defined by Kabat, Chothia, IMTG, etc.) from a mouse antibody, they can also be made with fewer mouse CDRs and/or less than the complete mouse CDR sequence (e.g. a functional fragment of a CDR). Thus, in embodiments only part of the donor CDRs, namely the subset of CDR residues required for binding, termed the SDRs, is incorporated into the humanized antibody.

Typically a humanized antibody can include (i) a light chain comprising at least one CDR (often three CDRs) from a mouse antibody (also referred to herein as a mouse CDR) and a human variable region framework; and (ii) a heavy chain comprising at least one CDR (often three CDRs) from the mouse antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In embodiments, the humanized antibody includes a light chain as described in (i), a heavy chain as described in (ii) together with a light chain human constant region and a heavy chain constant region.

In embodiments, the humanized antibody includes a human constant region. In embodiments, the humanized antibody is selected from an IgG (including IgG1, IgG2, IgG3, IgG4), IgM, IgA (IgA1, IgA2), IgD, and IgE isotype.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

Other approaches to design humanized antibodies may also be used to achieve the same result as the methods in U.S. Pat. Nos. 5,530,101 and 5,585,089 described above, for example, “super-humanization” (see Tan et al. J. Immunol. 169: 1119, 2002, and U.S. Pat. No. 6,881,557).

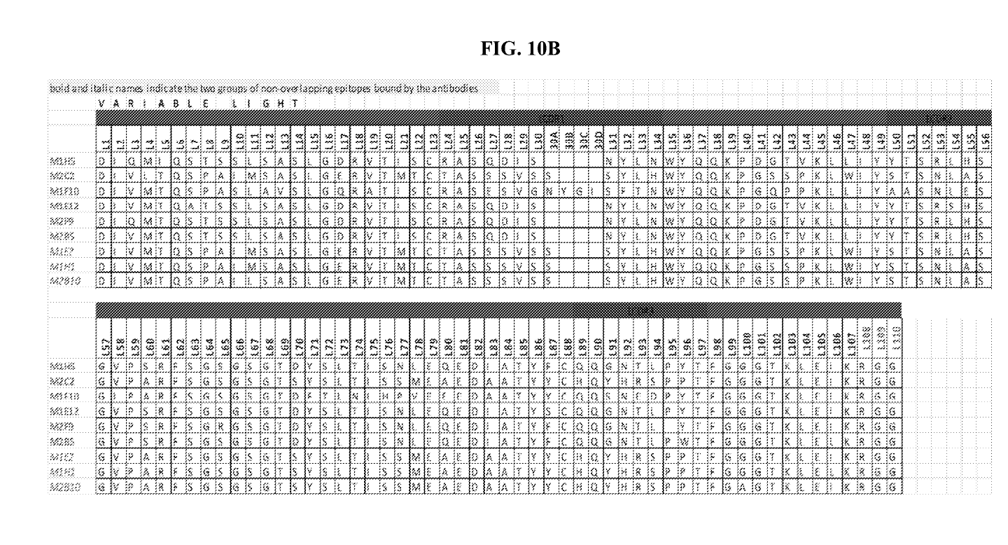

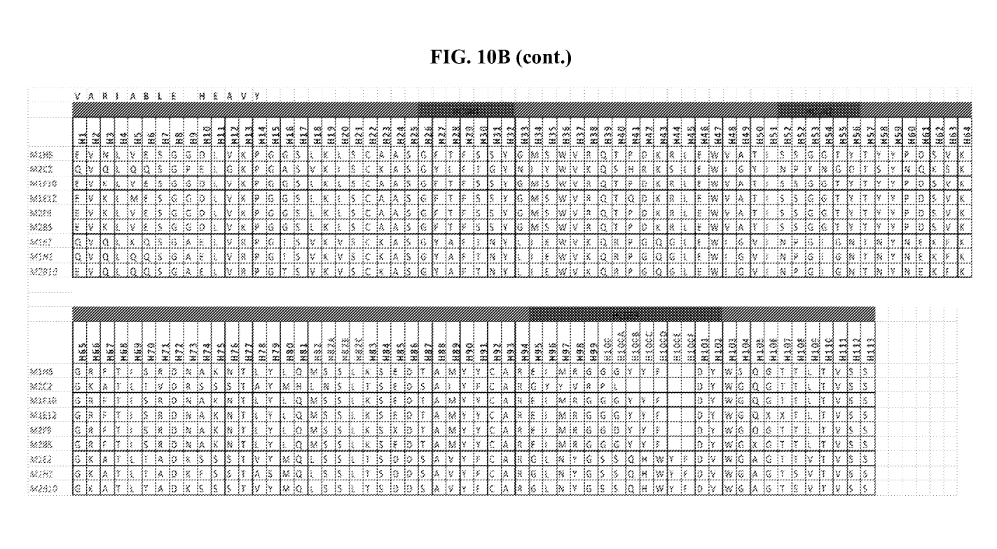

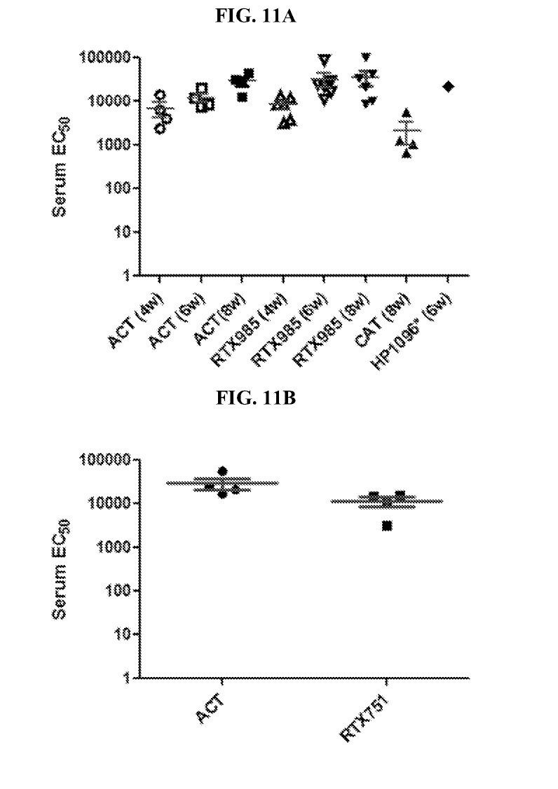

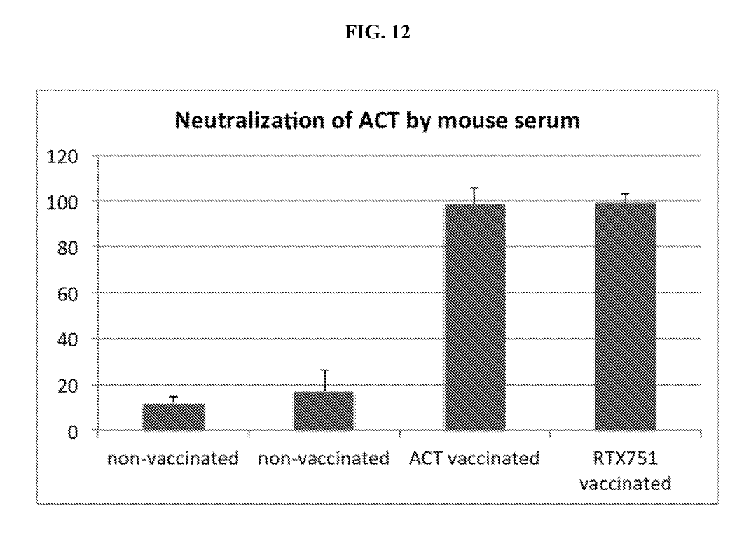

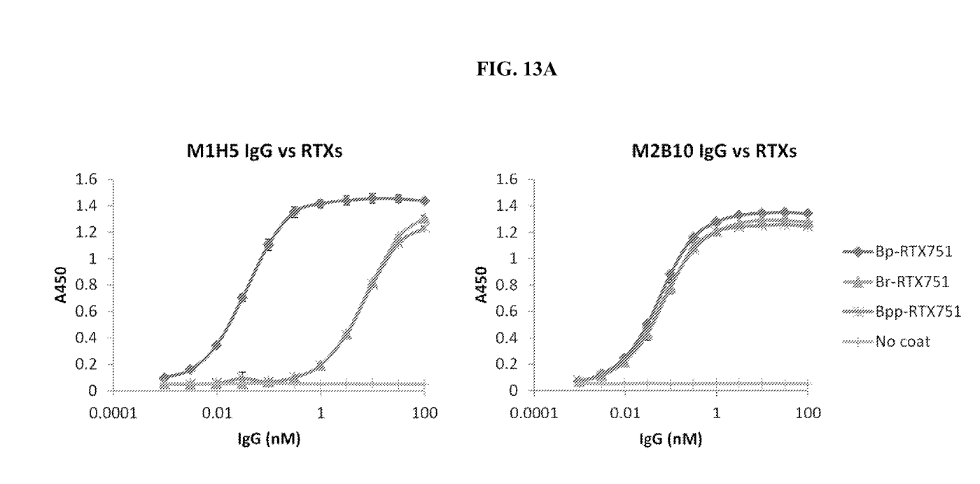

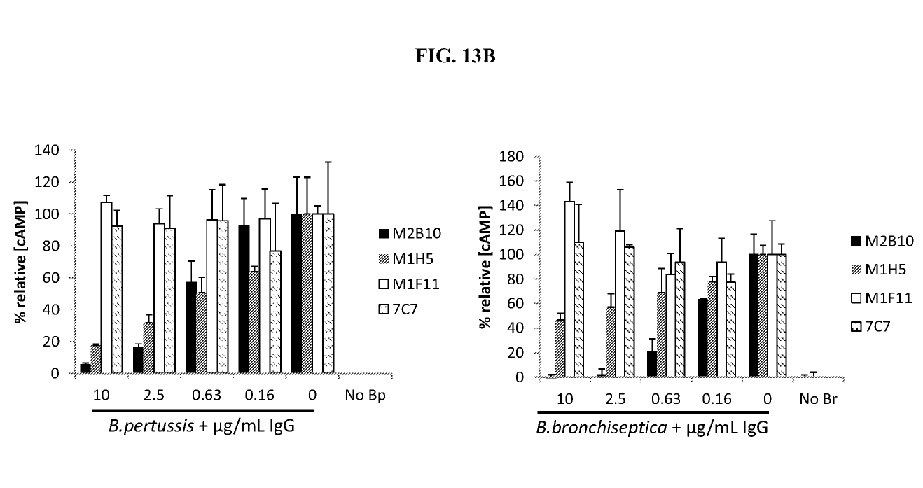

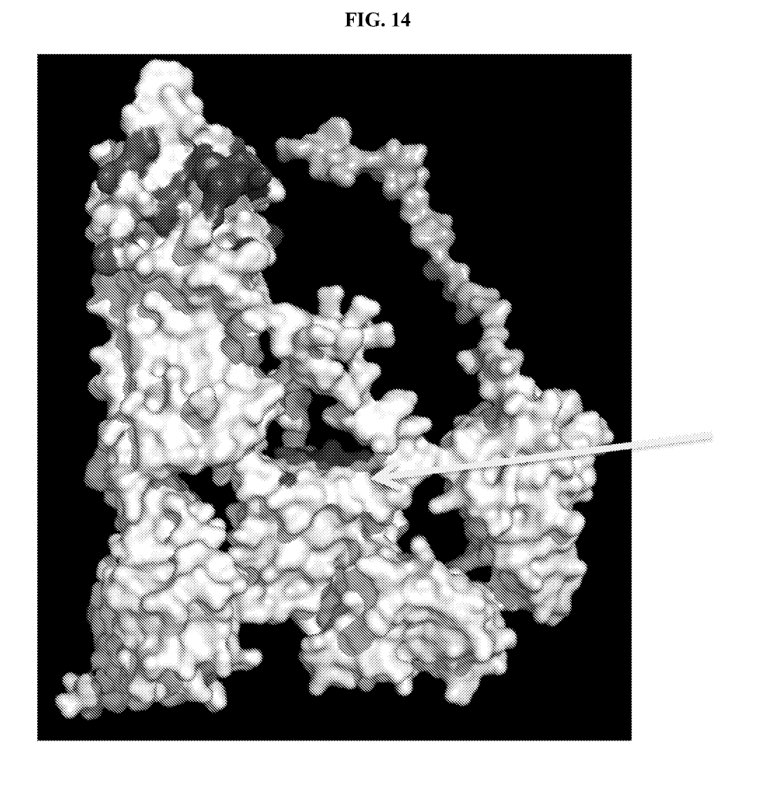

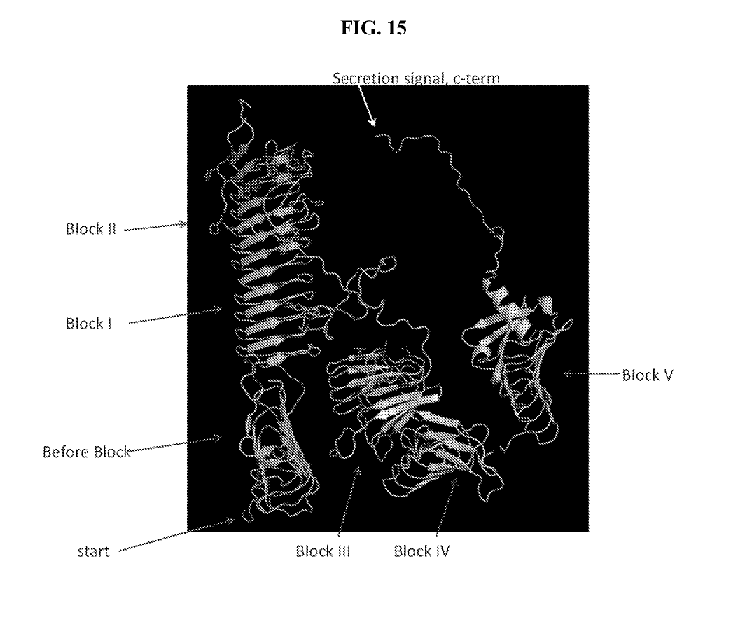

In embodiments, an antibody as described herein includes a humanized heavy chain and a humanized light chain. As described above the variable regions of the heavy chain and the light chain of an antibody include complementarity determining regions (CDRs). CDRs are defined as regions within an antibody that are directly involved in antigen binding. Proceeding from the amino-terminus, these regions are designated CDR H1, CDR H2 and CDR H3 for the heavy chain, and CDR L1, CDR L2, and CDR L3 for the light chain, respectively. The CDRs are held in place by more conserved framework regions (FRs). Proceeding from the amino-terminus, these regions are designated FR H1, FR H2, FR H3, and FR H4 for the heavy chain and FR L1, FR L2, FR L3, and FR L4, for the light chain, respectively. For humanized antibodies, one or more of the CDRs are derived from a donor antibody (also referred to herein as a donor CDR, such as a mouse CDR), whereas the FRs are of human origin. The locations of CDR and FR regions and the numbering system can be defined by any system known in the art, e.g., Kabat, Chothia, or IMGT (see, e.g., the website at bioinforg.uk/abs or Lefranc et al. (2009) Nucl. Acids Res. 26:D1006). The numbering and CDRs shown in FIG. 10 are based on Kabat.

D. Antibody Binding Assays

The specificity and affinity of the binding can be defined in terms of the comparative dissociation constants (Kd) of an for a target antigen, as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

An antibody will typically bind with a Kd of less than about 1000 nM, e.g., less than 250, 100, 50, 20 or lower nM. In embodiments, the Kd of the antibody is less than 15, 10, 5, or 1 nM. In embodiments, the Kd is 1-100 nM, 0.1-50 nM, 0.1-10 nM, or 1-20 nM. The value of the dissociation constant (Kd) can be determined by well-known methods, and can be computed even for complex mixtures by methods as disclosed, e.g., in Caceci et al., Byte (1984) 9:340-362.

Affinity of an antibody for a target antigen can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The Kd, Kon, and Koff can also be determined using surface plasmon resonance (SPR), e.g., as measured by using a Biacore T100 system. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptavidin (SA) sensor chip. The other interactant is then contacted with the chip and detected, e.g., as described in Abdessamad et al. (2002) Nuc. Acids Res. 30:e45.

The ability of a particular antibody to recognize the same epitope as another antibody can be determined by the ability of one antibody to competitively inhibit binding of a second antibody to an antigen, e.g., Bordetella ACT or a fragment thereof. Any of a number of competitive binding assays can be used to measure competition between two antibodies to the same antigen. An exemplary assay is a Biacore assay. Briefly, in these assays, binding sites can be mapped in structural terms by testing the ability of interactants, e.g. different antibodies, to inhibit the binding of another. Two consecutive antibody samples are injected in sufficient concentration to determine if the antibodies compete for binding of the same binding epitope.

Antibodies can be differentiated by the epitope to which they bind using a sandwich ELISA assay. This is carried out by using a capture antibody (e.g., such as an antibody listed in Table 2 or FIG. 10) to coat the surface of a well. A sub-saturating concentration of tagged-antigen is then added to the capture surface. The antigen will be bound to the antibody through a specific antibody:epitope interaction. After washing, a second antibody labeled with a detectable moiety (e.g., HRP, with the labeled antibody being defined as the detection antibody) is added to the ELISA. If this antibody recognizes the same epitope as the capture antibody it will be unable to bind to the target antigen, as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

### E. Methods of Making Antibodies

For preparation of the presently described antibodies, e.g., recombinant or monoclonal antibodies, techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell or phage display library, e.g., the genes encoding a monoclonal antibody can be cloned from a hybridoma or library and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)). Techniques for the production of single chain antibodies or recombinant antibodies (U.S. Pat. No. 4,946,778, U.S. Pat. No. 4,816,567) can be adapted to produce the presently described antibodies. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Any of the presently disclosed antibodies can also be included in a bispecific antibody that isable to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} and V_{L} region as described herein may optionally comprise a methionine at the N-terminus.

As noted above, the antibody can be produced in various formats, including as a Fab, a Fab′, a F(ab′)₂, a scFv, or a dAB. The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. An antibody of the invention can also include a human constant region. See, e.g., Fundamental Immunology (Paul ed., 4d ed. 1999); Bird, et al., Science 242:423 (1988); and Huston, et al., Proc. Natl. Acad. Sci. USA 85:5879 (1988).

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

Techniques for conjugating detectable agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

For labeled antibodies, the label can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY derivatives. Fluorescent dyes are discussed, for example, in U.S. Pat. No. 4,452,720, U.S. Pat. No. 5,227,487, and U.S. Pat. No. 5,543,295.

### IV. Diagnostic and Therapeutic Compositions and Methods

Any of the presently disclosed antibodies can be used for in vitro diagnostic or monitoring methods, e.g., using cells or tissue from a patient sample. A labeled antibody can also be provided to an individual in vivo to determine the applicability of an intended therapy. For example, a labeled antibody may be used to detect the severity of bacterial infection in an affected area.

Effective neutralization, e.g., for treatment, generally relies on an appropriately high affinity, based on a slow kinetic off-rate. If a given antibody off-rate is slower than the clearance time of the antibody-toxin complex in human sera, then the antibody can effectively remove toxin stoichiometrically. Even if clearance rates are slower than antibody off-rates, higher affinity antibodies will be better able to neutralize smaller amounts of a toxin. Thus, high affinity antibodies can be used at much lower doses, reducing the chance for side effects such as unwanted immune reactions to the antibody therapeutic reagent. Antibodies with dissociation constants (Kd) of about 70 nM or lower are generally used therapeutically.

One of skill will appreciate that the nature of the pharmaceutical composition and route of administration will depend in part on the condition being treated.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

A pharmaceutical composition comprising an antibody described herein can be administered, alone or in combination with other suitable components, can be made into aerosol formulations (“nebulized”) to be administered via inhalation. Aerosol formulations can be placed into pressurized acceptable propellants, such as dichlorodifluoromethane, propane, nitrogen, etc.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Antibodies are typically administered by parenteral or intravenous administration. The formulations of compounds can be presented in unit-dose or multi-dose sealed containers, such as ampules and vials.

Injection solutions and suspensions can be prepared from sterile powders, granules, and tablets of the kind previously described. Methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions for administration typically comprises an antibody as described herein (a neutralizing anti-ACT antibody) dissolved in a pharmaceutically acceptable carrier, e.g., an aqueous carrier. A variety of aqueous carriers can be used, e.g., buffered saline. These solutions are sterile and generally free of undesirable matter. These compositions may be sterilized by conventional, well known sterilization techniques. The compositions can contain pharmaceutically acceptable auxiliary substances as required to approximate physiological conditions such as pH adjusting and buffering agents, for example, sodium acetate, sodium chloride, potassium chloride, calcium chloride, sodium lactate, etc. The concentration of active agent in these formulations can vary widely, and will be selected primarily based on fluid volumes, viscosities, body weight and the like in accordance with the particular mode of administration selected and the patient's needs.

The formulation may also provide additional active compounds, including, e.g., antibacterial agents. The active ingredients can be prepared as sustained-release preparations (e.g., semi-permeable matrices of solid hydrophobic polymers (e.g., polyesters, hydrogels (for example, poly (2-hydroxyethyl-methacrylate), poly-vinylalcohol, polylactides). Antibodies and associated compounds can also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

The compositions can be administered for therapeutic or prophylactic treatments. In therapeutic applications, compositions are administered to a patient exposed to Bordetella in a “therapeutically effective dose.” Amounts effective for this use will depend upon the disorder to be treated, the route of administration, the severity of the condition, and the general state of the patient's health. Single or multiple administrations of the compositions may be administered depending on the dosage and frequency as required and tolerated by the patient. The presently described compositions can be administered to humans and other animals, particularly mammals. Thus the methods are applicable to both human therapy and veterinary applications. Other known cancer therapies can be used in combination with the methods of the invention. For example, the compositions for use as described herein can also be used to increase a patient's immune response to Bordetella.

Combination therapies contemplate coadministration, using separate formulations or a single pharmaceutical formulation, and consecutive administration in either order.

To determine a therapeutically effective dose, a low dose of an anti-Bordetella antibody can be initially administered to the individual, and the dose can be incrementally increased until the condition of the individual begins to improve. For example, the initial dosage can be about 0.001 mg/kg to about 1 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used at later time points if the condition of the individual does not change at the lowest dose. As noted above, one of skill will appreciate that a number of variables must be considered when determining a therapeutically effective dose. The dose administered to a patient should be sufficient to effect a beneficial therapeutic response in the patient over time. The size of the dose also will be determined by the existence, nature, and extent of any adverse side-effects that accompany the administration of a particular composition (or combination therapy) in a particular patient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and public database entries cited herein are hereby incorporated by reference in their entireties for all purposes.

### V. Vaccines and Methods of Immunizing

### Definitions

“Bordetella adenylate cyclase toxin protein” or “Bordetella ACT protein” is used according to its common, ordinary meaning and refers to proteins of the same or similar names and functional fragments and homologs thereof. The term includes recombinant or naturally occurring forms of Bordetella ACT protein (e.g., UniProt accession number C8C508; SEQ ID NO:28), or variants thereof that maintain Bordetella ACT protein activity (e.g. within at least 30%, 40%, 50%, 60%, 70%, 80%, 90%, 95%, or 100% as compared to SEQ ID NO:28). The term includes recombinant or naturally occurring forms of Bordetella ACT protein or variants thereof that have sequence identity to SEQ ID NO:28 (e.g., about 65%, 70%, 75%, 80%, 85%, 90%, 95%, 99% or 100% identity to SEQ ID NO:28). Bordetella ACT protein may refer to variants having mutated amino acid residues that modulate (e.g. increase or decrease when compared to Bordetella ACT protein) Bordetella ACT protein activity, expression, cellular targeting, or protein translocation. As provided herein Bordetella ACT protein includes, without limitation, ACT proteins of Bordetella pertussis, Bordetella bronchioseptica, Bordetella ansorpii, Bordetella avium, Bordetella hinzii, Bordetella holmesii, Bordetellapetrii, Bordetella trematum or Bordetella parapertussis. Thus, in embodiments, the Bordetella adenylate cyclase toxin protein has the sequence of SEQ ID NO:28, SEQ ID NO:29 or SEQ ID NO:30.

The terms “α_{M}β₂ integrin” or “CD11B” as referred to herein include any of the recombinant or naturally-occurring forms of the Cluster of Differentiation 11B (CD11B) also known as Integrin alpha M (ITGAM), heterodimeric integrin alpha-M beta-2 (α_{M}β2) molecule, macrophage-1 antigen (Mac-1) or complement receptor 3 (CR3), or variants or homologs thereof that maintain CD11B activity (e.g., within at least 50%, 80%, 90%, 95%, 96%, 97%, 98%, 99% or 100% activity compared to CD11B). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD11B protein. In embodiments, the CD11B protein is substantially identical to the protein identified by the UniProt reference number P11215 or a variant or homolog having substantial identity thereto. In embodiments, the CD11B protein is substantially identical to the protein identified by the UniProt reference number P05555 or a variant or homolog having substantial identity thereto.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to a reference sequence. In embodiments, the reference sequence is the sequence of amino acid residue 700-1076 of Bordetella adenylate cyclase toxin protein. In embodiments, the reference sequence is SEQ ID NO:28. In embodiments, the reference sequence is SEQ ID NO:29. In embodiments, the reference sequence is SEQ ID NO:30. In embodiments, the comparison to the reference sequence is a sequence alignment between the given amino acid or polynucleotide sequence and the reference sequence.

The sequence of the recombinant Bordetella adenylate cyclase toxin polypeptide as described herein corresponds to residues corresponding to positions 700-1706 of the Bordetella adenylate cyclase toxin protein or an immunogenic fragment thereof. One skilled in the art will immediately recognize the identity and location of residues corresponding to positions 700-1706 of the Bordetella adenylate cyclase toxin protein in other Bordetella adenylate cyclase toxin proteins with different numbering systems. For example, by performing a simple sequence alignment with Bordetella adenylate cyclase toxin protein the identity and location of residues corresponding to positions 700-1706 of the Bordetella adenylate cyclase toxin protein are identified in other Bordetella adenylate cyclase toxin proteins (e.g., Bordetella pertussis, Bordetella bronchioseptica or Bordetella parapertussis).

### Vaccine Compositions

In one aspect a vaccine composition is provided. The composition includes a recombinant Bordetella adenylate cyclase toxin polypeptide consisting essentially of a sequence corresponding to amino acid residues 700-1706 of Bordetella adenylate cyclase toxin protein or an immunogenic fragment thereof and a pharmaceutically acceptable vaccine excipient. The recombinant Bordetella adenylate cyclase toxin polypeptide or immunogenic fragment thereof provided herein are immunogenic polypeptides that upon administration to a human patient or animal generate antibodies that specifically bind to the Bordetella adenylate cyclase toxin protein. An immunogenic peptide is capable of inducing an immunological response against itself upon administration to a mammal, optionally in conjunction with an adjuvant.

The term “consisting essentially of” in this context refers to a recombinant Bordetella adenylate cyclase toxin polypeptide including a sequence corresponding to amino acid residues 700-1706 of Bordetella adenylate cyclase toxin protein or an immunogenic fragment thereof and no other sequence that is independently capable of generating antibodies that specifically bind to the Bordetella adenylate cyclase toxin protein or a portion of the Bordetella adenylate cyclase toxin protein.

In embodiments, the recombinant Bordetella adenylate cyclase toxin polypeptide consists of a sequence corresponding to amino acid residues 700-1706 of Bordetella adenylate cyclase toxin protein or an immunogenic fragment thereof and a pharmaceutically acceptable vaccine excipient. Where the Bordetella adenylate cyclase toxin polypeptide consists of a sequence corresponding to amino acid residues 700-1706 of Bordetella adenylate cyclase toxin protein or an immunogenic fragment thereof, the recombinant polypeptide includes a sequence corresponding to amino acid residues 700-1706 of Bordetella adenylate cyclase toxin protein or an immunogenic fragment thereof and no other sequence. Thus, in embodiments, the sequence corresponds to amino acid residues 700-1706. In embodiments, the sequence corresponds to amino acid residues 705-1706. In embodiments, the sequence corresponds to amino acid residues 710-1706. In embodiments, the sequence corresponds to amino acid residues 715-1706. In embodiments, the sequence corresponds to amino acid residues 720-1706. In embodiments, the sequence corresponds to amino acid residues 725-1706. In embodiments, the sequence corresponds to amino acid residues 730-1706. In embodiments, the sequence corresponds to amino acid residues 735-1706. In embodiments, the sequence corresponds to amino acid residues 740-1706. In embodiments, the sequence corresponds to amino acid residues 745-1706. In embodiments, the sequence corresponds to amino acid residues 750-1706. In embodiments, the sequence corresponds to amino acid residues 751-1706. In embodiments, the sequence corresponds to amino acid residues 752-1706. In embodiments, the sequence corresponds to amino acid residues 753-1706. In embodiments, the sequence corresponds to amino acid residues 754-1706. In embodiments, the sequence corresponds to amino acid residues 755-1706. In embodiments, the sequence corresponds to amino acid residues 756-1706. In embodiments, the sequence corresponds to amino acid residues 757-1706. In embodiments, the sequence corresponds to amino acid residues 758-1706. In embodiments, the sequence corresponds to amino acid residues 759-1706. In embodiments, the sequence corresponds to amino acid residues 760-1706.

In embodiments, the sequence corresponds to amino acid residues 765-1706. In embodiments, the sequence corresponds to amino acid residues 770-1706. In embodiments, the sequence corresponds to amino acid residues 775-1706. In embodiments, the sequence corresponds to amino acid residues 780-1706. In embodiments, the sequence corresponds to amino acid residues 785-1706. In embodiments, the sequence corresponds to amino acid residues 790-1706. In embodiments, the sequence corresponds to amino acid residues 795-1706. In embodiments, the sequence corresponds to amino acid residues 800-1706. In embodiments, the sequence corresponds to amino acid residues 805-1706. In embodiments, the sequence corresponds to amino acid residues 810-1706. In embodiments, the sequence corresponds to amino acid residues 815-1706. In embodiments, the sequence corresponds to amino acid residues 820-1706. In embodiments, the sequence corresponds to amino acid residues 825-1706. In embodiments, the sequence corresponds to amino acid residues 830-1706. In embodiments, the sequence corresponds to amino acid residues 835-1706. In embodiments, the sequence corresponds to amino acid residues 840-1706. In embodiments, the sequence corresponds to amino acid residues 845-1706. In embodiments, the sequence corresponds to amino acid residues 850-1706. In embodiments, the sequence corresponds to amino acid residues 855-1706. In embodiments, the sequence corresponds to amino acid residues 860-1706. In embodiments, the sequence corresponds to amino acid residues 865-1706. In embodiments, the sequence corresponds to amino acid residues 870-1706. In embodiments, the sequence corresponds to amino acid residues 875-1706. In embodiments, the sequence corresponds to amino acid residues 880-1706. In embodiments, the sequence corresponds to amino acid residues 885-1706. In embodiments, the sequence corresponds to amino acid residues 890-1706. In embodiments, the sequence corresponds to amino acid residues 895-1706.

In embodiments, the sequence corresponds to amino acid residues 900-1706. In embodiments, the sequence corresponds to amino acid residues 910-1706. In embodiments, the sequence corresponds to amino acid residues 920-1706. In embodiments, the sequence corresponds to amino acid residues 930-1706. In embodiments, the sequence corresponds to amino acid residues 940-1706. In embodiments, the sequence corresponds to amino acid residues 950-1706. In embodiments, the sequence corresponds to amino acid residues 960-1706. In embodiments, the sequence corresponds to amino acid residues 970-1706. In embodiments, the sequence corresponds to amino acid residues 975-1706. In embodiments, the sequence corresponds to amino acid residues 980-1706. In embodiments, the sequence corresponds to amino acid residues 981-1706. In embodiments, the sequence corresponds to amino acid residues 982-1706. In embodiments, the sequence corresponds to amino acid residues 983-1706. In embodiments, the sequence corresponds to amino acid residues 984-1706. In embodiments, the sequence corresponds to amino acid residues 985-1706. In embodiments, the sequence corresponds to amino acid residues 986-1706. In embodiments, the sequence corresponds to amino acid residues 987-1706. In embodiments, the sequence corresponds to amino acid residues 988-1706. In embodiments, the sequence corresponds to amino acid residues 989-1706. In embodiments, the sequence corresponds to amino acid residues 990-1706. In embodiments, the sequence corresponds to amino acid residues 995-1706. In embodiments, the sequence corresponds to amino acid residues 996-1706. In embodiments, the sequence corresponds to amino acid residues 997-1706. In embodiments, the sequence corresponds to amino acid residues 998-1706. In embodiments, the sequence corresponds to amino acid residues 999-1706.

In embodiments, the sequence corresponds to amino acid residues 1000-1706. In embodiments, the sequence corresponds to amino acid residues 1001-1706. In embodiments, the sequence corresponds to amino acid residues 1002-1706. In embodiments, the sequence corresponds to amino acid residues 1003-1706. In embodiments, the sequence corresponds to amino acid residues 1004-1706. In embodiments, the sequence corresponds to amino acid residues 1005-1706. In embodiments, the sequence corresponds to amino acid residues 1006-1706. In embodiments, the sequence corresponds to amino acid residues 1007-1706. In embodiments, the sequence corresponds to amino acid residues 1008-1706. In embodiments, the sequence corresponds to amino acid residues 1009-1706. In embodiments, the sequence corresponds to amino acid residues 1010-1706. In embodiments, the sequence corresponds to amino acid residues 1020-1706. In embodiments, the sequence corresponds to amino acid residues 1030-1706. In embodiments, the sequence corresponds to amino acid residues 1040-1706. In embodiments, the sequence corresponds to amino acid residues 1050-1706. In embodiments, the sequence corresponds to amino acid residues 1060-1706. In embodiments, the sequence corresponds to amino acid residues 1070-1706. In embodiments, the sequence corresponds to amino acid residues 1080-1706. In embodiments, the sequence corresponds to amino acid residues 1090-1706. In embodiments, the sequence corresponds to amino acid residues 1091-1706. In embodiments, the sequence corresponds to amino acid residues 1092-1706. In embodiments, the sequence corresponds to amino acid residues 1093-1706. In embodiments, the sequence corresponds to amino acid residues 1094-1706. In embodiments, the sequence corresponds to amino acid residues 1095-1706. In embodiments, the sequence corresponds to amino acid residues 1096-1706. In embodiments, the sequence corresponds to amino acid residues 1097-1706. In embodiments, the sequence corresponds to amino acid residues 1098-1706. In embodiments, the sequence corresponds to amino acid residues 1099-1706. In embodiments, the sequence corresponds to amino acid residues 1100-1706.

In embodiments, the sequence corresponds to amino acid residues 700-1705. In embodiments, the sequence corresponds to amino acid residues 705-1705. In embodiments, the sequence corresponds to amino acid residues 710-1705. In embodiments, the sequence corresponds to amino acid residues 715-1705. In embodiments, the sequence corresponds to amino acid residues 720-1705. In embodiments, the sequence corresponds to amino acid residues 725-1705. In embodiments, the sequence corresponds to amino acid residues 730-1705. In embodiments, the sequence corresponds to amino acid residues 735-1705. In embodiments, the sequence corresponds to amino acid residues 740-1705. In embodiments, the sequence corresponds to amino acid residues 745-1705. In embodiments, the sequence corresponds to amino acid residues 750-1705. In embodiments, the sequence corresponds to amino acid residues 751-1705. In embodiments, the sequence corresponds to amino acid residues 752-1705. In embodiments, the sequence corresponds to amino acid residues 753-1705. In embodiments, the sequence corresponds to amino acid residues 754-1705. In embodiments, the sequence corresponds to amino acid residues 755-1705. In embodiments, the sequence corresponds to amino acid residues 756-1705. In embodiments, the sequence corresponds to amino acid residues 757-1705. In embodiments, the sequence corresponds to amino acid residues 758-1705. In embodiments, the sequence corresponds to amino acid residues 759-1705. In embodiments, the sequence corresponds to amino acid residues 760-1705.

In embodiments, the sequence corresponds to amino acid residues 765-1705. In embodiments, the sequence corresponds to amino acid residues 770-1705. In embodiments, the sequence corresponds to amino acid residues 775-1705. In embodiments, the sequence corresponds to amino acid residues 780-1705. In embodiments, the sequence corresponds to amino acid residues 785-1705. In embodiments, the sequence corresponds to amino acid residues 790-1705. In embodiments, the sequence corresponds to amino acid residues 795-1705. In embodiments, the sequence corresponds to amino acid residues 800-1705. In embodiments, the sequence corresponds to amino acid residues 805-1705. In embodiments, the sequence corresponds to amino acid residues 810-1705. In embodiments, the sequence corresponds to amino acid residues 815-1705. In embodiments, the sequence corresponds to amino acid residues 820-1705. In embodiments, the sequence corresponds to amino acid residues 825-1705. In embodiments, the sequence corresponds to amino acid residues 830-1705. In embodiments, the sequence corresponds to amino acid residues 835-1705. In embodiments, the sequence corresponds to amino acid residues 840-1705. In embodiments, the sequence corresponds to amino acid residues 845-1705. In embodiments, the sequence corresponds to amino acid residues 850-1705. In embodiments, the sequence corresponds to amino acid residues 855-1705. In embodiments, the sequence corresponds to amino acid residues 860-1705. In embodiments, the sequence corresponds to amino acid residues 865-1705. In embodiments, the sequence corresponds to amino acid residues 870-1705. In embodiments, the sequence corresponds to amino acid residues 875-1705. In embodiments, the sequence corresponds to amino acid residues 880-1705. In embodiments, the sequence corresponds to amino acid residues 885-1705. In embodiments, the sequence corresponds to amino acid residues 890-1705. In embodiments, the sequence corresponds to amino acid residues 895-1705.

In embodiments, the sequence corresponds to amino acid residues 900-1705. In embodiments, the sequence corresponds to amino acid residues 910-1705. In embodiments, the sequence corresponds to amino acid residues 920-1705. In embodiments, the sequence corresponds to amino acid residues 930-1705. In embodiments, the sequence corresponds to amino acid residues 940-1705. In embodiments, the sequence corresponds to amino acid residues 950-1705. In embodiments, the sequence corresponds to amino acid residues 960-1705. In embodiments, the sequence corresponds to amino acid residues 970-1705. In embodiments, the sequence corresponds to amino acid residues 975-1705. In embodiments, the sequence corresponds to amino acid residues 980-1705. In embodiments, the sequence corresponds to amino acid residues 981-1705. In embodiments, the sequence corresponds to amino acid residues 982-1705. In embodiments, the sequence corresponds to amino acid residues 983-1705. In embodiments, the sequence corresponds to amino acid residues 984-1705. In embodiments, the sequence corresponds to amino acid residues 985-1705. In embodiments, the sequence corresponds to amino acid residues 986-1705. In embodiments, the sequence corresponds to amino acid residues 987-1705. In embodiments, the sequence corresponds to amino acid residues 988-1705. In embodiments, the sequence corresponds to amino acid residues 989-1705. In embodiments, the sequence corresponds to amino acid residues 990-1705. In embodiments, the sequence corresponds to amino acid residues 995-1705. In embodiments, the sequence corresponds to amino acid residues 996-1705. In embodiments, the sequence corresponds to amino acid residues 997-1705. In embodiments, the sequence corresponds to amino acid residues 998-1705. In embodiments, the sequence corresponds to amino acid residues 999-1705.

In embodiments, the sequence corresponds to amino acid residues 1000-1705. In embodiments, the sequence corresponds to amino acid residues 1001-1705. In embodiments, the sequence corresponds to amino acid residues 1002-1705. In embodiments, the sequence corresponds to amino acid residues 1003-1705. In embodiments, the sequence corresponds to amino acid residues 1004-1705. In embodiments, the sequence corresponds to amino acid residues 1005-1705. In embodiments, the sequence corresponds to amino acid residues 1006-1705. In embodiments, the sequence corresponds to amino acid residues 1007-1705. In embodiments, the sequence corresponds to amino acid residues 1008-1705. In embodiments, the sequence corresponds to amino acid residues 1009-1705. In embodiments, the sequence corresponds to amino acid residues 1010-1705. In embodiments, the sequence corresponds to amino acid residues 1020-1705. In embodiments, the sequence corresponds to amino acid residues 1030-1705. In embodiments, the sequence corresponds to amino acid residues 1040-1705. In embodiments, the sequence corresponds to amino acid residues 1050-1705. In embodiments, the sequence corresponds to amino acid residues 1060-1705. In embodiments, the sequence corresponds to amino acid residues 1070-1705. In embodiments, the sequence corresponds to amino acid residues 1080-1705. In embodiments, the sequence corresponds to amino acid residues 1090-1705. In embodiments, the sequence corresponds to amino acid residues 1091-1705. In embodiments, the sequence corresponds to amino acid residues 1092-1705. In embodiments, the sequence corresponds to amino acid residues 1093-1705. In embodiments, the sequence corresponds to amino acid residues 1094-1705. In embodiments, the sequence corresponds to amino acid residues 1095-1705. In embodiments, the sequence corresponds to amino acid residues 1096-1705. In embodiments, the sequence corresponds to amino acid residues 1097-1705. In embodiments, the sequence corresponds to amino acid residues 1098-1705. In embodiments, the sequence corresponds to amino acid residues 1099-1705. In embodiments, the sequence corresponds to amino acid residues 1100-1705.

In embodiments, the sequence corresponds to amino acid residues 700-1600. In embodiments, the sequence corresponds to amino acid residues 705-1600. In embodiments, the sequence corresponds to amino acid residues 710-1600. In embodiments, the sequence corresponds to amino acid residues 715-1600. In embodiments, the sequence corresponds to amino acid residues 720-1600. In embodiments, the sequence corresponds to amino acid residues 725-1600. In embodiments, the sequence corresponds to amino acid residues 730-1600. In embodiments, the sequence corresponds to amino acid residues 735-1600. In embodiments, the sequence corresponds to amino acid residues 740-1600. In embodiments, the sequence corresponds to amino acid residues 745-1600. In embodiments, the sequence corresponds to amino acid residues 750-1600. In embodiments, the sequence corresponds to amino acid residues 751-1600. In embodiments, the sequence corresponds to amino acid residues 752-1600. In embodiments, the sequence corresponds to amino acid residues 753-1600. In embodiments, the sequence corresponds to amino acid residues 754-1600. In embodiments, the sequence corresponds to amino acid residues 755-1600. In embodiments, the sequence corresponds to amino acid residues 756-1600. In embodiments, the sequence corresponds to amino acid residues 757-1600. In embodiments, the sequence corresponds to amino acid residues 758-1600. In embodiments, the sequence corresponds to amino acid residues 759-1600. In embodiments, the sequence corresponds to amino acid residues 760-1600.

In embodiments, the sequence corresponds to amino acid residues 765-1600. In embodiments, the sequence corresponds to amino acid residues 770-1600. In embodiments, the sequence corresponds to amino acid residues 775-1600. In embodiments, the sequence corresponds to amino acid residues 780-1600. In embodiments, the sequence corresponds to amino acid residues 785-1600. In embodiments, the sequence corresponds to amino acid residues 790-1600. In embodiments, the sequence corresponds to amino acid residues 795-1600. In embodiments, the sequence corresponds to amino acid residues 800-1600. In embodiments, the sequence corresponds to amino acid residues 805-1600. In embodiments, the sequence corresponds to amino acid residues 810-1600. In embodiments, the sequence corresponds to amino acid residues 815-1600. In embodiments, the sequence corresponds to amino acid residues 820-1600. In embodiments, the sequence corresponds to amino acid residues 825-1600. In embodiments, the sequence corresponds to amino acid residues 830-1600. In embodiments, the sequence corresponds to amino acid residues 835-1600. In embodiments, the sequence corresponds to amino acid residues 840-1600. In embodiments, the sequence corresponds to amino acid residues 845-1600. In embodiments, the sequence corresponds to amino acid residues 850-1600. In embodiments, the sequence corresponds to amino acid residues 855-1600. In embodiments, the sequence corresponds to amino acid residues 860-1600. In embodiments, the sequence corresponds to amino acid residues 865-1600. In embodiments, the sequence corresponds to amino acid residues 870-1600. In embodiments, the sequence corresponds to amino acid residues 875-1600. In embodiments, the sequence corresponds to amino acid residues 880-1600. In embodiments, the sequence corresponds to amino acid residues 885-1600. In embodiments, the sequence corresponds to amino acid residues 890-1600. In embodiments, the sequence corresponds to amino acid residues 895-1600.

In embodiments, the sequence corresponds to amino acid residues 900-1600. In embodiments, the sequence corresponds to amino acid residues 910-1600. In embodiments, the sequence corresponds to amino acid residues 920-1600. In embodiments, the sequence corresponds to amino acid residues 930-1600. In embodiments, the sequence corresponds to amino acid residues 940-1600. In embodiments, the sequence corresponds to amino acid residues 950-1600. In embodiments, the sequence corresponds to amino acid residues 960-1600. In embodiments, the sequence corresponds to amino acid residues 970-1600. In embodiments, the sequence corresponds to amino acid residues 975-1600. In embodiments, the sequence corresponds to amino acid residues 980-1600. In embodiments, the sequence corresponds to amino acid residues 981-1600. In embodiments, the sequence corresponds to amino acid residues 982-1600. In embodiments, the sequence corresponds to amino acid residues 983-1600. In embodiments, the sequence corresponds to amino acid residues 984-1600. In embodiments, the sequence corresponds to amino acid residues 985-1600. In embodiments, the sequence corresponds to amino acid residues 986-1600. In embodiments, the sequence corresponds to amino acid residues 987-1600. In embodiments, the sequence corresponds to amino acid residues 988-1600. In embodiments, the sequence corresponds to amino acid residues 989-1600. In embodiments, the sequence corresponds to amino acid residues 990-1600. In embodiments, the sequence corresponds to amino acid residues 995-1600. In embodiments, the sequence corresponds to amino acid residues 996-1600. In embodiments, the sequence corresponds to amino acid residues 997-1600. In embodiments, the sequence corresponds to amino acid residues 998-1600. In embodiments, the sequence corresponds to amino acid residues 999-1600.

In embodiments, the sequence corresponds to amino acid residues 1000-1600. In embodiments, the sequence corresponds to amino acid residues 1001-1600. In embodiments, the sequence corresponds to amino acid residues 1002-1600. In embodiments, the sequence corresponds to amino acid residues 1003-1600. In embodiments, the sequence corresponds to amino acid residues 1004-1600. In embodiments, the sequence corresponds to amino acid residues 1005-1600. In embodiments, the sequence corresponds to amino acid residues 1006-1600. In embodiments, the sequence corresponds to amino acid residues 1007-1600. In embodiments, the sequence corresponds to amino acid residues 1008-1600. In embodiments, the sequence corresponds to amino acid residues 1009-1600. In embodiments, the sequence corresponds to amino acid residues 1010-1600. In embodiments, the sequence corresponds to amino acid residues 1020-1600. In embodiments, the sequence corresponds to amino acid residues 1030-1600. In embodiments, the sequence corresponds to amino acid residues 1040-1600. In embodiments, the sequence corresponds to amino acid residues 1050-1600. In embodiments, the sequence corresponds to amino acid residues 1060-1600. In embodiments, the sequence corresponds to amino acid residues 1070-1600. In embodiments, the sequence corresponds to amino acid residues 1080-1600. In embodiments, the sequence corresponds to amino acid residues 1090-1600. In embodiments, the sequence corresponds to amino acid residues 1091-1600. In embodiments, the sequence corresponds to amino acid residues 1092-1600. In embodiments, the sequence corresponds to amino acid residues 1093-1600. In embodiments, the sequence corresponds to amino acid residues 1094-1600. In embodiments, the sequence corresponds to amino acid residues 1095-1600. In embodiments, the sequence corresponds to amino acid residues 1096-1600. In embodiments, the sequence corresponds to amino acid residues 1097-1600. In embodiments, the sequence corresponds to amino acid residues 1098-1600. In embodiments, the sequence corresponds to amino acid residues 1099-1600. In embodiments, the sequence corresponds to amino acid residues 1100-1600.

As described herein the polypeptide provided herein may consist essentially of a sequence corresponding to amino acid residues 700-1706 of Bordetella adenylate cyclase toxin protein or an immunogenic fragment thereof. In embodiments, the sequence includes a detectable moiety. In embodiments, the detectable moiety is a peptide. In embodiments, the detectable moiety is a poly-histidine peptide. In embodiments, the sequence is SEQ ID NO:31, SEQ ID NO:32, SEQ ID NO:33, SEQ ID NO:34, SEQ ID NO:35, SEQ ID NO:36, SEQ ID NO:37 or SEQ ID NO:38. In embodiments, the sequence is SEQ ID NO:31. In embodiments, the sequence is SEQ ID NO:32. In embodiments, the sequence is SEQ ID NO:33. In embodiments, the sequence is SEQ ID NO:34. In embodiments, the sequence is SEQ ID NO:35. In embodiments, the sequence is SEQ ID NO:36. In embodiments, the sequence is SEQ ID NO:37. In embodiments, the sequence is SEQ ID NO:38.

The sequences of the recombinant Bordetella adenylate cyclase toxin polypeptide provided herein may include an acylated amino acid residue. An “acylated amino acid residue” as provided herein refers to an amino acid covalently attached to an acyl group (R—C(O)—). Thus, in embodiments, the sequence includes an acylated amino acid residue. In embodiments, the sequence includes an acylated amino acid residue at a position corresponding to K983 or K860. In further embodiments, the Bordetella adenylate cyclase toxin polypeptide binds α_{M}β₂ integrin. In embodiments, the sequence includes an acylated amino acid residue at a position corresponding to K860. In embodiments, the sequence includes an acylated amino acid residue at a position corresponding to K983. In embodiments, the acylated residue is a lysine.

In embodiments, the Bordetella adenylate cyclase toxin polypeptide binds α_{M}β₂ integrin. In embodiment, where the Bordetella adenylate cyclase toxin polypeptide binds α_{M}β₂ integrin, the polypeptide or immunogenic fragment thereof is internalized if bound to α_{M}β₂ integrin expressed on a cell (e.g., a leukocyte involved in the innate immune system, including monocyte, granulocyte, macrophage, and natural killer cell). In embodiments, where the Bordetella adenylate cyclase toxin polypeptide binds α_{M}β₂ integrin, the polypeptide or immunogenic fragment thereof is capable of competitively inhibiting the binding of a α_{M}β₂ integrin antibody (i.e., an antibody that specifically binds α_{M}β₂ integrin). Thus, the Bordetella adenylate cyclase toxin polypeptide that binds α_{M}β₂ integrin may recognize the same epitope as a α_{M}β₂ integrin antibody. Any of a number of competitive binding assays can be used to measure competition between the polypeptide and the antibody to the same antigen (α_{M}β₂ integrin). An exemplary assay is a Biacore® assay. Briefly, in these assays, binding sites can be mapped in structural terms by testing the ability of interactants, e.g. different polypeptides to inhibit the binding of another. Other conventional immunoassays known in the art can be used in the present invention. For example, antibodies and polypeptides can be differentiated by the epitope to which they bind using a sandwich ELISA assay. This is carried out by using a capture antibody (e.g. a mouse α_{M}β₂ integrin antibody) to coat the surface of a well. A sub-saturating concentration of tagged-antigen (α_{M}β₂ integrin) is then added to the capture surface. This protein will be bound to the antibody through a specific antibody:epitope interaction. After washing a recombinant Bordetella adenylate cyclase toxin polypeptide as provided herein (e.g. a polypeptide capable of binding α_{M}β₂ integrin), which has been covalently linked to a detectable moiety (e.g., HRP, polyhistidine (HIS-tag)) is added to the ELISA. If the polypeptide recognizes the same epitope as the capture antibody it will be unable to bind to the target protein as that particular epitope will no longer be available for binding and no signal will be detected. Alternatively, the α_{M}β₂ integrin may be allowed to bind the Bordetella adenylate cyclase toxin polypeptide as provided herein, thereby forming a receptor-polypeptide complex and preventing subsequent interaction with a α_{M}β₂ integrin specific antibody.

In embodiments, the Bordetella adenylate cyclase toxin polypeptide as provided herein competes with an antibody that is capable of binding α_{M}β₂ integrin. In embodiments, where the Bordetella adenylate cyclase toxin polypeptide competes with an antibody (competitor antibody) for binding α_{M}β₂ integrin, the Bordetella adenylate cyclase toxin polypeptide inhibits (completely or partially) binding of the competitor antibody to a measurable extent. The inhibition of binding may be measured by any of the methods described above. In general, a Bordetella adenylate cyclase toxin polypeptide is considered to competitively inhibit binding of a competitor antibody, if binding of the competitor antibody to the antigen (α_{M}β₂ integrin) is reduced by at least 30%, usually at least about 40%, 50%, 60% or 75%, and often by at least about 90%, in the presence of the Bordetella adenylate cyclase toxin polypeptide using any of the assays described above. In embodiments. where the Bordetella adenylate cyclase toxin polypeptide binds α_{M}β2 integrin, the polypeptide binds with a Kd of less than 1 μM. In embodiments. where the Bordetella adenylate cyclase toxin polypeptide binds α_{M}β2 integrin, the polypeptide binds with a Kd of less than 0.5 μM.

The sequences of the recombinant Bordetella adenylate cyclase toxin polypeptide provided herein may not include an acylated amino acid residue. Thus, in embodiments, the sequence is a non-acylated sequence. In embodiments, the sequence includes a non-acylated amino acid residue at a position corresponding to K983 or K860. In embodiments, the sequence includes a non-acylated amino acid residue at a position corresponding to K983. In embodiments, the sequence includes a non-acylated amino acid residue at a position corresponding to K860. In embodiments, the non-acylated residue is a lysine. In embodiments, the Bordetella adenylate cyclase toxin polypeptide does not bind α_{M}β₂ integrin. In further, embodiments the sequence is a non-acylated sequence.

The Bordetella adenylate cyclase toxin polypeptide provided herein may not bind α_{M}β₂ integrin. In embodiments, where the Bordetella adenylate cyclase toxin polypeptide does not bind α_{M}β₂ integrin, the polypeptide or immunogenic fragment thereof is not internalized if bound to α_{M}β₂ integrin expressed on the surface of a cell (e.g., leukocyte involved in the innate immune system, including monocyte, granulocyte, macrophage, and natural killer cell). In embodiments, where the Bordetella adenylate cyclase toxin polypeptide does not bind α_{M}β₂ integrin, the polypeptide or immunogenic fragment thereof does not competitively inhibit the binding of a α_{M}β₂ integrin antibody. Thus, the Bordetella adenylate cyclase toxin polypeptide that does not bind α_{M}β₂ integrin may not recognize the same epitope as a α_{M}β₂ integrin antibody. In embodiments. where the Bordetella adenylate cyclase toxin polypeptide does not bind α_{M}β₂ integrin, the polypeptide does not bind with a Kd of less than 10 μM. In embodiments. where the Bordetella adenylate cyclase toxin polypeptide does not bind α_{M}β₂ integrin, the polypeptide does not bind with a Kd of less than 5 μM. In embodiments. where the Bordetella adenylate cyclase toxin polypeptide does not bind α_{M}β₂ integrin, the polypeptide does not bind with a Kd of less than 1 μM.

### Methods of Immunizing

In one aspect a method for immunizing a subject in need thereof against Bordetella pertussis is provided. The method includes administering to the subject an effective amount of the vaccine as provided herein including embodiments thereof. In embodiments, the subject is a human. Method provided is performed under conditions such that antibodies directed to the recombinant Bordetella adenylate cyclase toxin polypeptide or immunogenic fragment thereof are produced. In embodiments, the antibodies prevent binding of Bordetella adenylate cyclase toxin protein of Bordetella pertussis, Bordetella bronchioseptica or Bordetella parapertussis to α_{M}β₂ integrin. In embodiments, the antibodies are capable of binding adenylate cyclase toxin of Bordetella pertussis, Bordetella bronchioseptica or Bordetella parapertussis. In embodiments, the recombinant Bordetella adenylate cyclase toxin polypeptide fragment forms part of a vaccine.

The recombinant Bordetella adenylate cyclase toxin polypeptides as provided herein may be formulated and introduced as a vaccine through oral, intradermal, intramuscular, intraperitoneal, intravenous, subcutaneous, intranasal, and via scarification (scratching through the top layers of skin, e.g., using a bifurcated needle) or any other standard route of immunization. Vaccine formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient or any other oral composition as listed above. Alternatively, the vaccines may be administered parenterally as injections (intravenous, intramuscular or subcutaneous). Alternatively, recombinant Bordetella adenylate cyclase toxin polypeptides of the present invention may be encapsulated in liposomes and administered via injection. Commercially available liposome delivery systems are available from Novavax, Inc. of Rockville, Md., commercially available under the name Novasomes™. These liposomes are specifically formulated for immunogen delivery. The amount of recombinant Bordetella adenylate cyclase toxin polypeptides used in a vaccine can depend upon a variety of factors including the route of administration, species, and use of booster administration. However, a person of ordinary skill in the art would immediately recognize appropriate and/or equivalent doses looking at dosages of approved whopping cough vaccines for guidance.

The term “adjuvant” refers to a compound that when administered in conjunction with an antigen (e.g. recombinant Bordetella adenylate cyclase toxin polypeptide) augments the immune response to the antigen, but when administered alone does not generate an immune response to the antigen. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the recombinant Bordetella adenylate cyclase toxin polypeptides as provided herein, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa) or monophosphoryl lipid-A (/MPL) formulated with cationic dimethyldioctadecylammonium (DDA) liposomes (DDA/MPL). Further contemplated are oligodeoxynucleotides containing specific CpG motifs (CpG ODNs such as ODN 1826 and ODN 2006), synthetic analogs of double-stranded RNA such as Poly (I:C), Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, N Y, 1995); U.S. Pat. No. 5,057,540), (Aquila BioPharmaceuticals, Framingham, Mass.), imidazoquinolines (i.e. imiquimod, gardiquimod and R848) and curdlan as a TH17 stimulating adjuvant. Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other examples of adjuvants are aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, Mont.) containing 2% squalene, 0.2% Tween 80, and one or more bacterial cell wall components from the group consisting of monophosphoryllipid A (MPL), trehalose dimycolate (TDM), and cell wall skeleton (CWS), preferably MPL+CWS (Detox™).

Other adjuvants contemplated for the invention are saponin adjuvants, such as Stimulon™ (QS-21, Aquila, Framingham, Mass.) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

Agents for inducing an immune response can be administered by parenteral, topical, intravenous, oral, subcutaneous, intraarterial, intracranial, intraperitoneal, intranasal or intramuscular means for prophylactic and/or therapeutic treatment. The most typical route of administration of an immunogenic agent is subcutaneous although other routes can be equally effective. The next most common route is intramuscular injection. This type of injection is most typically performed in the arm or leg muscles.

For parenteral administration, agents of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Polypeptides can be administered in the form of a depot injection or implant preparation which can be formulated in such a manner as to permit a sustained release of the active ingredient. An exemplary composition comprises recombinant Bordetella adenylate cyclase toxin polypeptide at 5 mg/ml, formulated in aqueous buffer consisting of 50 mM L-histidine, 150 mM NaCl, adjusted to pH 6.0 with HCl. Composition for parenteral administration are typically substantially sterile, isotonic and manufactured under GMP conditions of the FDA or similar body.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

### VI. Methods of Treatment

In another aspect, a method of preventing or treating whooping cough in a subject in need thereof is provided. The method includes administering to the subject a therapeutically effective amount of the vaccine as provided herein including embodiments thereof.

The term “therapeutically effective amount” means the amount of the recombinant Bordetella adenylate cyclase toxin polypeptide that will elicit the biological or medical response of a cell, tissue, system, or animal, such as a human, that is being sought by the researcher, veterinarian, medical doctor or other treatment provider.

The term “vaccine excipient” as provided herein refers to any inactive substance appropriate as carried for active ingredients (e.g., a recombinant Bordetella adenylate cyclase toxin polypeptide) of a vaccine composition. Non limiting examples of a vaccine excipient include, acetone, alcohol, anhydrous lactose, castor oil, cellulose acetate phthalate, dextrose, D-fructose, D-mannose, FD&C Yellow #6 aluminum lake dye, fetal bovine serum, human serum albumin, magnesium stearate, micro crystalline cellulose, plasdone C, Polacrilin potassium, potassium phosphate, sodium bicarbonate, sucrose, aluminum hydroxide, amino acids, benzethonium chloride, formaldehyde, inorganic salts and sugars, vitamins, citric acid, lactose, glycerin, iron ammonium citrate, magnesium sulfate, beta-propiolactone, egg protein, neomycin, nonylphenol ethoxylate, polymyxin, thimerosal, and potassium phosphate. Acceptable vaccine excipients are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids such as glycine, carbohydrates, chelating agents, sugars, and other standard ingredients known to those skilled in the art (Remington's Pharmaceutical Science 16^{th} edition, Osol, A. Ed. 1980). The polypeptide is typically present at a concentration of 0.1-100 mg/ml, e.g., 1-10 mg/ml or 10-50 mg/ml, for example 5, 10, 20, 30, 40, 50 or 60 mg/ml.

A pharmaceutical composition including a recombinant Bordetella adenylate cyclase toxin polypeptide as described herein can be administered by a variety of methods known in the art. The route and/or mode of administration vary depending upon the desired results. In embodiments, administration is intravenous, intramuscular, intraperitoneal, or subcutaneous, or administered proximal to the site of the target. Pharmaceutically acceptable vaccine excipients can be suitable for intravenous, intramuscular, subcutaneous, parenteral, spinal or epidermal administration (e.g., by injection or infusion).

Pharmaceutical compositions of the recombinant Bordetella adenylate cyclase toxin polypeptide can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the polypeptide is employed in the pharmaceutical compositions of the invention. The polypeptide can be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It may be advantageous to formulate the recombinant Bordetella adenylate cyclase toxin polypeptide in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of recombinant Bordetella adenylate cyclase toxin polypeptide calculated to produce the desired therapeutic effect in association with the required pharmaceutical excipient.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the polypeptide being employed, the duration of the treatment, other drugs, compounds and/or materials used in combination with the particular compositions employed, the age, sex, weight, condition, general health and prior medical history of the patient being treated, and like factors.

A physician or veterinarian can start doses of the recombinant Bordetella adenylate cyclase toxin polypeptide of the invention employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with a polypeptide, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

Recombinant Bordetella adenylate cyclase toxin polypeptides can be administered on multiple occasions. Intervals between single dosages can be weekly, monthly or yearly. Intervals can also be irregular as indicated by measuring blood levels of polypeptide in the patient. In some methods, dosage is adjusted to achieve a plasma polypeptide concentration of 1-1000 μg/ml and in some methods 25-300 μg/ml. Alternatively, polypeptides can be administered as a sustained release formulation, in which case less frequent administration is required. Dosage and frequency vary depending on the half-life of the polypeptide in the patient.

### VII. Examples

A. Experimental Procedures

### Molecular Cloning of ACT Domains for Soluble Expression

Individual ACT domains were expressed in the bacterial cytoplasm of E. coli with His₆-tags. To generate plasmids expressing only the catalytic domain (residues 1-373, 1-385 or 1-400), the corresponding coding regions were amplified from pT7CACT3 (29) by PCR, with common forward primer 5′-aggaaacaCATATGcagcaatcgcatcaggctgg-3′ (SEQ ID NO:19) and reverse primers 5′-actaGAATTCttacgaacgtccgctcggcacg-3′ (SEQ ID NO:20), 5′-cacaGAATTCttacgccggcaccgtttccagtacatc-3′ (SEQ ID NO:21), and 5′-cataGAATTCttactggcgttccactgcgcc-3′ (SEQ ID NO:22) respectively (restriction sites in uppercase and underlined). The amplified fragments were gel purified and double digested with NdeI and EcoRI and ligated into similarly digested pET28a vector. To generate plasmids expressing the RTX domains (residues 751-1706 or 985-1706), DNA fragments encoding these regions were amplified using forward primers 5′-tcacgaaCATATGgccaattcggacg-3′ (SEQ ID NO:23) and 5′-ctacggcCATATGacggagaatgtcca-3′ (SEQ ID NO:24), and common reverse primer 5′-ataGGATCCtcagcgccagttgacag-3′ (SEQ ID NO:25). The resulting PCR products were gel purified, double digested with NdeI and BamHI, and ligated into similarly digested pET28a vector.

To enhance folding and solubility, the hydrophobic domain, encompassing the region between the catalytic and RTX domains (residues 399-1096) was cloned into pMalc-5× vector (NEB) between NdeI and BamHI sites, downstream of the maltose binding protein (MBP). The primers for PCR were 5′-gggcgcaCATATGcgccaggattccggct-3′ (SEQ ID NO:26) and 5′-atcggcGGATCCttaatggtgatgatggtgatgggcgctggcctcggaaggctggtgcac-3′ (SEQ ID NO:27); with the bold nucleotides encoding a C-terminal His₆-tag. The cyaC gene was inserted downstream of the hydrophobic domain between the BamHI and HindIII sites, with an upstream ribosome binding to allow for co-expression.

### ACT and Domain Expression and Purification

Full-length ACT was expressed from the plasmid pT7CACT3 with co-expression of the palmitoylating enzyme CyaC in Escherichia coli strain XL-1 Blue (29). The holo-toxin was purified using a single-step calmodulin agarose affinity chromatography as described by Sebo et al. (2) Purified ACT was stored in 50 mM Tris, 8M urea, 2 mM EDTA, pH 8.0 at 4° C. for short term or −80° C. for long term storage. The protein concentration was determined by absorbance at 280 nm using a molecular extinction coefficient of 143590 M⁻¹cm⁻¹ as calculated from its amino acid sequence (30). ACT from BEI Resources was used as a reference for purity and toxicity.

The catalytic and RTX domains of ACT were expressed in E. coli strain BL21(DE3). Briefly, 250 mL of TB media were inoculated from starter cultures to an optical density at 600 nm (OD₆₀₀) of 0.05, grown at 37° C. until OD₆₀₀=0.3-0.6, at which time 0.4 mM IPTG (isopropyl-β-D-thiogalactopyranoside) was added to induce expression. After 4 h of room temperature growth, the cells were harvested, resuspended in Buffer A (50 mM Hepes, 250 mM NaCl, 2 mM CaCl₂, 40 mM imidazole, pH 8.0), and lysed with a French Press (Thermo Scientific). After a 20 min centrifugation step at 20,000 rpm (JA-20 rotor), the supernatant was applied to a HisTrap column on an AKTA FPLC (GE healthcare), followed by elution with a linear gradient of Buffer B (Buffer A+500 mM imidazole). The hydrophobic domain was expressed in E. coli strain BL21 as above, although purification included immobilized metal affinity chromatography (IMAC) resin followed by an MBPTrap affinity column (GE healthcare) and elution with 10 mM maltose.

### Biophysical Characterization

To assess the oligomeric status of ACT and its domains, fractions eluted from Histrap or IMAC were supplemented with either 100 μL of 100 mM EGTA or 100 mM HBSC (HBS+2 mM CaCl₂), incubated on ice for 1 hr, and loaded onto Superdex S200 column equilibrated with HBS (50 mM Hepes, 150 mM NaCl, pH 7.8) or HBSC, respectively. Running buffers were HBS or HBSC to ensure the absence or presence of calcium ion. To assess secondary structure characteristics, purified ACT domains were dialyzed into 10 mM Tris-H₂SO₄ pH8.0 and the concentrations were adjusted to 100 μg/mL. To observe the effect of calcium ions on protein conformation, CaCl₂ were added to a final concentration of 2 mM and incubated at room temperature for 1 h. CD spectra between 180-260 nm were collected on a J-815 CD Spectrometer (JASCO) at 25° C., at lnm intervals using a 1 mm rectangular cell. Each spectrum represents the average of 3 scans subtracted with the spectrum of buffers (with or without CaCl₂). Data were fitted with the CDSSTR program on the DichroWeb server to estimate the percentage of secondary structures (31).

### Murine Immunization

All protocols were approved by the University of Texas at Austin IACUC (protocol #2012-00068) and all mice were handled in accordance with IACUC guidelines. As a source for antibody libraries, two 6-week BALB/c mice were primed intraperitoneally (i.p.) with 17 μg of ACT (dialyzed against PBS to remove urea) in complete Freud's adjuvant (CFA). Four weeks later, the mice were bled through tail vein, and boosted subcutaneously with the same amount of PBS-dialyzed ACT in incomplete Freund's adjuvant (IFA). Two weeks later, the mice were sacrificed and blood collected by cardiac puncture. Spleens were removed sterilely, sliced into pieces and immediately immersed in 1 ml of cold RNALater solution. After soaking overnight at 4° C., the solution was removed and the spleens were stored at −80° C.

To assess the immunogenicity of individual ACT domains, 4-6 BALB/c mice per group were immunized subcutaneously with equal moles of ACT and individual domains (10 μg for ACT, 2.6 μg for 1-400, 6.7 μg for 399-1096, and 4.4 μg for 985-1706) in complete Freund's adjuvant. Four weeks later, the mice were boosted subcutaneously with the same amount of antigen in incomplete Freud's adjuvant, a process which was repeated at 6 and 8 weeks. Blood was collected before immunization, four weeks after the first injection, and two weeks after each boost. Anti-ACT antibody titers were determined by ELISA (described below), with titer defined as the 50% effective concentration (EC₅₀) from a 4-parameter logistic fitting to the ELISA data. Neutralization of ACT-induced cAMP intoxication of J774A.1 macrophage cells (ATCC #TIB-67) was tested with a 1:400 sera dilution.

### Phage Display Antibody Library Construction

Total RNA was extracted from frozen spleens with TRIZol (Invitrogen) and the RNeasy Mini Kit (QIAgen) or PureLink RNA kit (Invitrogen) according to the manufacturers' instructions. The quality and concentration of total RNA was assessed by agarose gel electrophoresis and A_{230:260:280} ratio (approximately 1:2:1 for pure RNA) measured by NanoDrop 2000 (Thermo Scientific). For first-strand cDNA synthesis, 5 μg of total RNA was used. To maximize diversity, two separate reactions were performed using combinations of Superscript II+d(T)₂₃ VN primer or Superscript III (Invitrogen)+random hexamer (Thermo Scientific), following manufacturers' instructions. The two sets of cDNA were pooled as template for amplification of the V_{L} and V_{H} repertoires using the primer sets and PCR conditions described by Krebber et al. (32). The PCR products were gel purified, with 10 ng each of V_{L} and V_{H} used as template in an overlap PCR to generate V_{L}-linker-V_{H} fragments (scFv). This product was gel purified and digested overnight with SfiI prior to directional ligation with similarly SfiI-digested pMopac24 vector (33). Ten individual electroporations were performed to transform XL1-Blue cells. The transformants were pooled and an aliquot was 10-fold serially diluted and plated to count library size; the rest were plated on eight 150 mm 2×YT agar plates (10 μg/mL tetracycline, 200 μg/mL ampicillin, and 2% glucose). After incubation overnight at 37° C., the bacterial lawns were scraped off in 2×YT medium and pooled to form the master library.

### Phage Production, Purification, and Panning

Aliquots of the master library was used to inoculate 250 mL 2×YT medium with 10 μg/mL tetracycline, 200 μg/mL ampicillin, and 2% glucose in 1 L flasks to an OD₆₀₀ of ˜0.1. The cultures were grown at 37° C. for 2-3 hours until the OD₆₀₀ reached ˜0.6, induced and rescued by adding 1 mM IPTG and M13K07 helper phage (MOI ˜20), incubated for 30 min without shaking at 37° C., and then returned to a shaking incubator at room temperature. Three hours after adding helper phage, the culture was supplemented with 50 μg/mL kanamycin prior to overnight incubation with shaking. Phage were then purified by double precipitation with 1/5 volume of precipitation solution (2.5M NaCl, 20% PEG-8000). The concentration of viable phage was assessed as colony forming units (cfu), with serially diluted phage added to log-phase XL1-Blue cells, followed by plating on 2×YT agar plate with 200 μg/mL ampicillin, and enumeration of colonies after overnight incubation.

Two rounds of panning were performed using ACT as bait. Eight ELISA plate wells (Costar) were coated with 50 μL of 2 μg/mL and 1 μg/mL ACT in PBS at 4° C. overnight for the first and second round respectively. Input phage (100 ul) were diluted into 900 ut of 5% non-fat milk in PBST (PBS, 0.05% Tween-20) and incubated for 1 h before transferring 100 μL to each of the 8 wells. After a 1 hour incubation at room temperature, followed by five (or ten for round 2) washes with PBST, bound phage were eluted with 100 μL per well of 0.1 N HCl for 10 min at RT. The eluted phage was pooled and immediately neutralized with 48 μL of 2M Tris base. Half of the output phages were added into 5 ml of log-phase XL1-Blue culture grown with 10 μg/mL tetracycline at 37° C. to retain the F plasmid, incubated for 30 min without shaking and 1 hr with shaking at 225 rpm in 37° C., then spun down and plated on six 150 mm 2YT agar plate (200 μg/mL ampicillin, 10 μg/mL tetracycline, and 2% glucose). After overnight incubation at 37° C., colonies or lawn were scraped, pooled, mixed thoroughly, and aliquots used for second round of panning as described above.

Input and output phage titers (colony forming unit, cfu) were determined by infecting and plating E. coli as described above. Sequence diversity was monitored throughout all steps by performing colony PCR of random colonies on the phage titration plates, followed by BstNI fingerprinting and agarose gel electrophoresis. Clones with unique fingerprints were confirmed by DNA sequencing.

To produce monoclonal phage clones from panning outputs, single colonies from output plates were inoculated into sterile 96-well plate containing 100 μL 2YT medium (2% glucose, 200 μg/mL ampicillin, and 10 μg/mL tetracycline), grown at 37° C. overnight with shaking. The next morning, 10 μL of the overnight culture was inoculated into another plate with 90 μL per well of fresh medium containing 0.25% glucose and antibiotics, grown at 37° C. for 3 hr, then 50 μL 2YT (200 μg/mL Amp, 3 mM IPTG, and M13K07 helper phage) was added and let shaking at room temperature for 3 hours, before adding 50 μL of 2YT (200 μg/mL ampicillin, 1 mM IPTG, 200 μg/mL kanamycin). The plate was then shaken at room temperature overnight.

### Antibody Expression and Purification

To convert phage-displayed scFvs to soluble single chain antibody fragments (scAbs), consisting of a variable light chain domain (V_{L}) connected via a flexible (Gly₄Ser)₄ to a variable heavy chain domain (V_{H}) followed by a human kappa constant domain to enhance expression and solubility, the scFv region was removed from pMopac24 phagemid vector by SfiI digestion, and directionally ligated into SfiI-digested pMopac54 plasmid (34). For scAb production, 100 ml of TB supplemented with 200 μg/mL ampicillin and 1% glucose were inoculated at OD₆₀₀=0.02, grown overnight at room temperature. The next morning, cells were pelleted at 5000 g for 10 min at room temperature, resuspended in 100 ml of TB medium with ampicillin but no glucose, and grown at room temperature for 1 h before induction with 1 mM IPTG. After another 4 hours, cells were harvested by centrifugation at 5000 g, 10 min, 4° C. Osmotic shock was performed as described (34). scAbs in the dialyzed shockates were purified by IMAC resin followed by size exclusion chromatography with a Superdex 200 column on FPLC (GE Healthcare). Protein concentrations were measured by BCA assays (Pierce) using a BSA standard with purity assessed by SDS-PAGE.

To convert scAbs into full-length IgG with enhanced stabilities and in vivo half-lives, the V_{L} and V_{H} genes were subcloned onto Igκ-Abvec and IgG-Abvec vectors as described by Smith et al. (35). For IgG production, paired Igκ-Abvec and IgG-Abvec plasmids were transiently transfected into CHO-K1 cells using Lipofectamine 2000 (Life Technologies) according to manufacturer's instructions. Culture media were collected at 1-2 day intervals, neutralized with 1M Tris pH 8.0, pooled, and IgG purified by ammonium sulfate precipitation followed by HiTrap Protein A column. The purity and presence of aggregates were assessed by SDS-PAGE and size-exclusion chromatography using a Superdex S200 column. The concentration was determined by A₂₈₀ using extinction coefficients calculated from deduced amino acid sequences (30).

### Analysis of Antibody Binding by ELISA

For monoclonal phage screening, the 96-well phage production plates described above were spun at 3000 g for 20 min, with 40 μl of supernatant transferred to a coated (2 μg/ml ACT in PBS) and blocked (5% nonfat milk in PBST, M-PBST) ELISA plate containing 60 μL M-PBST per well, and incubated at room temperature for 1 hr. After 4 washes with PBST, 50 μL of 1:2000 HRP-conjugated anti-M13 antibody (GE Healthcare) was added, and incubated 1 hr at room temperature. The plate was washed 4 times, 50 μL/well TMB substrate added and incubated at room temperature. The reaction was quenched by adding 504/well 1M HCl, and the absorbance at 450 nm recorded with a SpectraMax M5 (Molecular Devices). Wells with absorbance higher than 2-fold of background were identified for further characterization.

Binding assays to assess domain specificity and relative affinity of soluble scAb or IgG proteins were performed in a similar manner. Purified ACT (1 μg/mL) or domains (equimolar with ACT) were coated on ELISA plates, followed by blocking and serial dilutions of purified antibodies, and finally detection with goat anti-mouse IgG HRP-conjugated antibody (to detect murine antibodies), goat anti-human κ chain HRP-conjugated antibody (to detect scAbs) or goat anti-human IgG (Fc-specific) HRP-conjugated antibody (to detect recombinant IgG). For competition ELISA, the antibody of interest was used at a fixed concentration determined to yield 70-80% of the maximal signal, and mixed with an equal volume of serially diluted competitor antibody, followed by detection as above.

To assess the reactivity of sera from humans exposed to pertussis, purified ACT (1 μg/mL) or domains (equimolar concentrations as ACT) were coated on ELISA plates. Plates were blocked as above, human sera serially diluted in M-PBST, and bound antibodies detected with goat anti-human IgG (Fc-specific) HRP-conjugated antibody. Nine randomly selected samples were tested in duplicate. The absorbance value for each sample binding to a domain was normalized to that sample's signal on an ACT-coated well at a 100-fold dilution (in the linear dose-response range) as follows: [A_{450(domain coated well)}−A_{450(uncoated well)}]/[A_{450(ACT coated well)}−A_{450(uncoated well)}]. Human sera were obtained from Vanderbilt University Medical Center under a protocol approved by the local institutional review board (IRB 061262, 070258 and 090806). Use of those samples was approved by the University of Texas at Austin (2009-05-0096). The study was conducted in accordance with the Declaration of Helsinki, with written informed consent obtained from each participant prior to study entry. The original consent forms allowed for sample use in subsequent studies. CaCl₂ levels in all ELISA assays were maintained at >2 mM unless indicated and each assay was performed at least twice.

In Vitro cAMP Intoxication & Neutralization Assay

J774A.1 cells were grown in DMEM (Sigma) supplemented with 10% fetal bovine serum, 1 mM sodium pyruvate, and penicillin/streptomycin. To measure the cAMP intoxication of J774A.1 cells by ACT or antibody neutralization, J774A.1 cells were seeded at 4×10⁴/cm² in 24-well plates (Costar) one day before the assay. ACT alone or with antibodies was diluted in DMEM without supplements in a final volume of 1 mL; base DMEM media contains 1.8 mM CaCl₂. ACT was used at 125 ng/mL in all assays unless otherwise specified; antibodies were present at 160-fold molar excess. While ACT and antibody mixtures incubated at room temperature for 30 min, cells were washed twice with plain DMEM prior to addition of 480 μL of antibody-ACT solution to duplicate wells. The plate was incubated at 37° C. for 30 min in a CO₂ incubator, followed by two washes with cold PBS. Lysis solution (500 μL; 0.1N HCl, 0.1% Triton X-100) was added into each well, and plate was rocked on ice for 10 min. The lysates were transferred into 1.5 ml tubes and boiled for 10 min to inactivate ACT and cAMP hydrolyzing enzymes. To assess cAMP intoxication in CHO cells, ACT was used at 250 ng/mL, with all other assay conditions kept constant.

The resulting lysates were clarified by centrifugation at 13,000 g for 5 min. The supernatant was diluted 6-fold with 200 mM Hepes, 150 mM NaCl, 0.05% Tween, pH 8.0 prior to cAMP measurement using a competition ELISA as described by Karimova et al. (36). All assays were performed at least in duplicate, with cAMP concentrations normalized to total protein concentration in the lysates as measured by BCA assay (Pierce).

To evaluate ACT neutralization in the context of the whole bacterium, B. pertussis Tohama I was grown on Bordet Gengou agar plate supplemented with 15% defibrinated sheep blood (BD) at 37° C. for 4 to 5 days. Bacteria were then inoculated into modified synthetic Stainer-Scholte medium (SSM) and grown at 37° C. with shaking at 225 rpm for 20˜24 h, to an OD600 of 0.7˜1.0. Bacteria were pelleted by centrifugation at 5,000 g for 10 min, resuspended in PBS, and diluted to an OD₆₀₀ of 0.4 in DMEM+10% heat-inactivated (HI) FBS; then mixed with equal volume of serially diluted neutralizing or control IgGs in DMEM+10% HI-FBS, and incubated at room temperature for 20 min before addition to adherent J774A.1 cells and incubation of 1 hr at 37° C. Intracellular cAMP level was then measured as above.

### J774A.1 Cell Lysis Assay

Cell lysis was monitored by enzymatic activity of lactate dehydrogenease released into the medium upon cell lysis. J774A.1 cells were seeded 10⁵ cells/well in 96-well round-bottom plates one day before the assay. 100 μL each of ACT alone (0.5 μg/mL) or ACT pre-incubated with antibodies (10 μg/mL, 160-fold molar excess antibody) in plain DMEM were added to triplicate wells and incubated for 2 hrs at 37° C. Then the plate was centrifuged at 250 g for 5 min and LDH activity in the supernatants were measured by a colorimetric assay with the CytoTox 96 kit (Promega, Wis., USA). After subtracting the background signal from control wells without ACT, the sample absorbance at 490 nm was normalized to ACT-only controls as follows: 100%*[A_{490 (ACT+antibody)}−A_{490 (no ACT)}]/[A_{490 (ACT only)}−A_{490 (no ACT)}].

### Analysis of ACT-Integrin Binding by ELISA

Recombinant murine α_{M}β₂ integrin (R&D Systems) was coated at 1 μg/mL in PBS at 4° C. overnight, and blocked with M-PBST. ACT or purified domains were serially diluted and incubated for 1 h at room temperature, followed by detection with polyclonal rabbit anti-ACT antibody and HRP-conjugated goat anti-rabbit antibody. To assess the effect of antibodies on ACT binding to integrin, ACT (1 μg/mL) was mixed with equal volume of serially diluted antibody (5, 1.6, 0.5 and 0-fold molar excess) and incubated for 1 h at room temperature before transferring to blocked α_{M}β₂ ELISA plate. The bound ACT was detected as described above.

### Flow Cytometry Analysis of ACT Binding to Cells

ACT was dialyzed against HBSC (50 mM Hepes, 150 mM NaCl, 2 mM CaCl₂ pH 8.0) to remove urea, and then biotinylated with 100-fold molar excess of EZ-Link Sulfo-NHS-LC-Biotin (Thermo Scientific) at room temperature for 2-3 h before quenching with 1M Tris (pH 8.0) and dialysis against HBSC overnight in 4° C. Biotinylated ACT (B-ACT; 210 μL of 0.8 μg/mL) was incubated with equal volume containing 60 μg/mL purified scAb (M2B10, M1H5, M1F11, and M1C12) in DMEM+1% BSA at room temperature for 30 min. Then, 2004 of the incubated mixtures was added to 4×10⁵ washed J774A.1 cells in duplicate, and incubated on ice for 30 min to allow ACT binding but not internalization. After two washes with FACS buffer (HBSC+2% FBS), 2004 of 1:500 phycoerythrin (PE)-conjugated Streptavidin (BioLegend) was used to detect cell-associated biotinylated ACT. After 20 min incubation on ice and three washes, the cells were finally resuspended in 6004 FACS buffer and analyzed on an LSR Fortessa II. Data analysis was performed with Flowjo software (V10).

B. Results

### Example 1: ACT is Prone to Aggregation and Proteolysis

The native ACT holotoxin secreted from B. pertussis readily aggregates at the bacterial surface and is prone to proteolytic degradation (28), while recombinant toxin expressed in the E. coli cytoplasm forms inclusion bodies (37-39). To increase the yield of purified protein, 8M urea is used to extract the aggregated protein from bacterial cell pellets; even so the protein is highly susceptible to proteolysis with early reports observing enzymatic activity in 43 and 45 kDa fragments (40). Efforts to remove urea, such as dialysis and dilution, result in significant aggregation and fragmentation (41).

As a result, standard purification protocols include solubilization of the cell pellet with urea, followed by calmodulin affinity or sequential anionic and hydrophobic interaction chromatographic steps followed by storage in 8M urea. Assays using the toxin call for dialysis or dilution of urea-solubilized ACT into assay media immediately before experimentation, most likely resulting in an ensemble of fully and partially folded ACT molecules and uncertainty regarding the exact concentration of active toxin molecules. ACT purified in our lab exhibited similar activity as that described by Eby et al (42): 125 ng/mL produced 10,000˜40,000 pmoles cAMP per mg of J774A.1 total cellular protein in 30 min at 37° C.

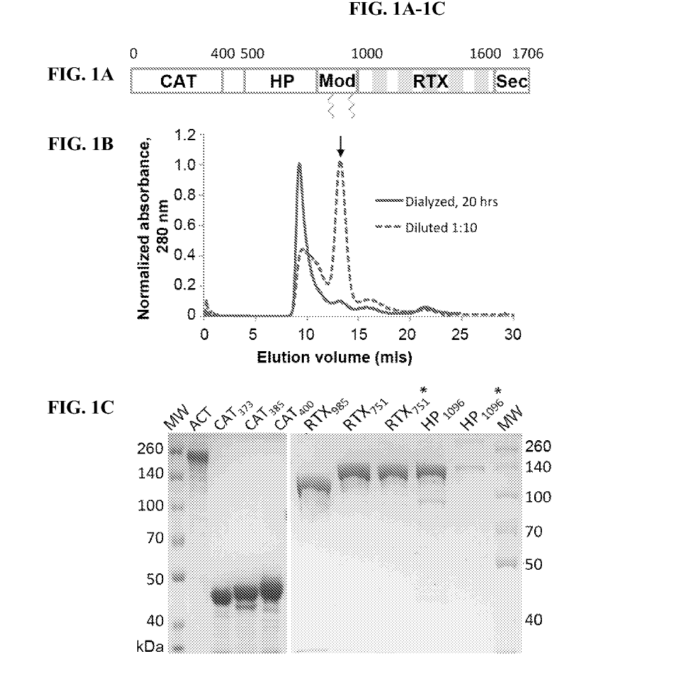

However, dialysis or dilution of ACT into buffer with no urea led to protein that was poorly behaved and retained by the SEC column (<5% of protein applied to the column eluted). In contrast, overnight dialysis into HBSC containing 1 M urea resulted in high molecular weight aggregates (˜600 kDa) that eluted off the SEC column. An alternate refolding approach, rapid 10-fold dilution into HBSC (final urea concentration 0.8 M) followed immediately by SEC, yielded a broader aggregate peak and a smaller peak corresponding to the expected size for monomer (FIG. 1B). The different monomer yields may reflect a time-dependent aggregation process or the presence of folding intermediates with different aggregation propensities in the two refolding procedures. A recent report reiterated the challenges of refolding ACT (43).

### Example 2: Individual ACT Domains are Biophysically Superior to ACT

To identify which, if any, ACT domains are predominantly recognized by polyclonal antibody responses, we expressed individual domains in E. coli with affinity tags to facilitate purification (Table 1). Based on prior reports (18,29,44-46), the n-terminal catalytic domains (residues 1-373 [CAT₃₇₃], 1-385 [CAT₃₈₅] and 1-400 [CAT₄₀₀]) and c-terminal RTX domains (residues 751-1706 [RTX₇₅₁] and 985-1706 [RTX₉₈₅]) were cloned into the pET28a vector for cytoplasmic expression with n-terminal His₆ tags to facilitate purification. In our hands, RTX₄₈₂₋₁₇₀₆ was poorly soluble and purified inefficiently; instead we selected RTX₉₈₅ as the largest fragment to exclude both acylation sites but retaining the N-terminus before the first Gly-Asp rich repeat. To enhance solubility, the hydrophobic domain (residues 399-1096 [HP₁₀₉₆], encompassing the region between the catalytic and RTX domains) was fused downstream of maltose binding protein (MBP), with a c-terminal His₆ tag and di-cistronic expression of the specific acylating enzyme CyaC (indicated by *). After cytoplasmic expression of each construct and cell lysis, a one-step affinity chromatography with a HisTrap column yielded ˜5-80 mg protein per liter culture with >90% purity as determined by SDS-PAGE (FIG. 1C). For HP-MBP-fusion proteins, a second chromatographic step with an MBPTrap column was required to reach a similar level of purity, although purity, proteolysis and solubility issues persisted.

TABLE 1
Biophysical analysis of ACT constructs

| | Theoretical MW | SEC calc. MW (kDa) | | Melting temp. |
|---|---|---|---|---|
| Domain | (kDa) | +Ca | +EGTA | (° C.) |
| ACT | 177 | >600 | ND | ND |
| CAT₄₀₀ | 45 | 40 | 40 | 43.3 |
| HP₁₀₉₆ | 118 | >600 | >600 | ND |
| HP₁₀₉₆* | 118 | >600 | >600 | ND |
| RTX₉₈₅ | 77 | 78 | 380 | 40.7 |
| RTX₇₅₁ | 102 | 108 | 115 | 57.7 |
| RTX₇₅₁* | 102 | 91 | >600 | 56.8 |

ACT and various derivatives were purified and subjected to size exclusion chromatography in the presence of 2 mM calcium or an excess of EGTA to chelate free calcium ions. The expected molecular weight for each construct is noted, as is the observed size and thermal melting temperature of the major peak. Constructs noted with * were co-expressed with CyaC to acylate residues K860 and K983.

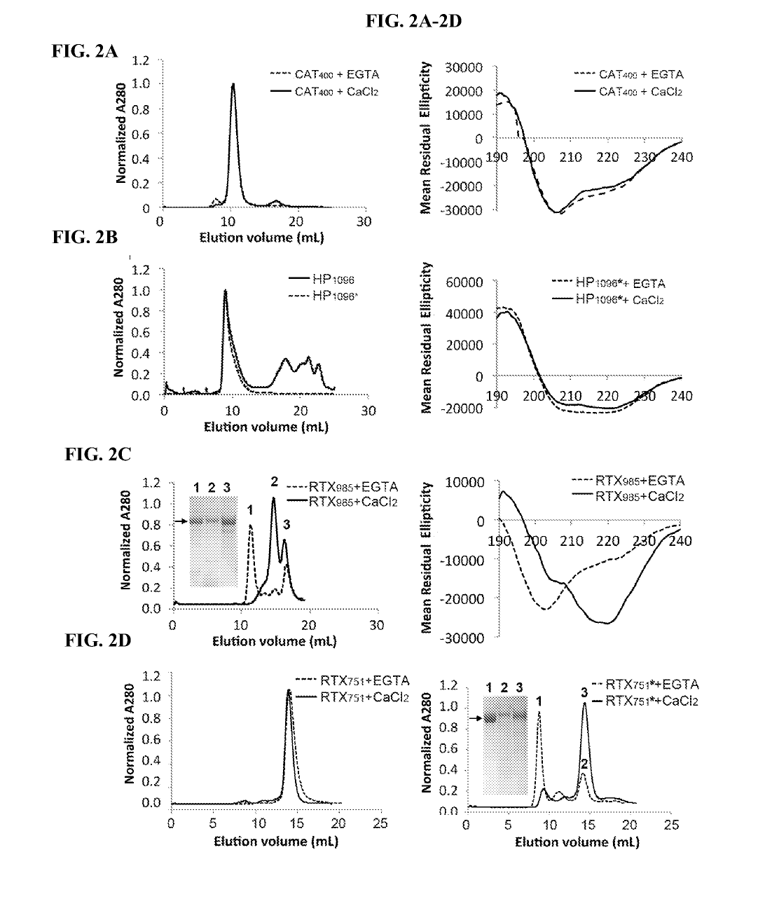

To determine whether the purified domains exhibited native-like structure and expected calcium-dependent structural changes, size exclusion chromatography (SEC) was used to assess the oligomeric state while circular dichroism (CD) spectroscopy was used to assess the secondary structure content. The catalytic domains eluted as a single peak of expected size (40 kDa) with the estimated composition of secondary structures (56% helix, 14% strands, 13% turns and 17% unordered) similar to that determined by X-ray crystallography (FIG. 2A) (18). While the catalytic domain formally encompasses residues 1-373, the construct encompassing residues 1-400 was selected for further use to include the neutralizing epitope recognized by antibody 3D1 (47).

The hydrophobic fusion proteins eluted as broad aggregate peaks when acylated or non-acylated. In the absence of acylation, multiple smaller peaks were observed, suggesting that acylation may stabilize folding of this domain and protect against proteolysis. In both cases, the CD spectra were not characteristic of unfolded or aggregated proteins (FIG. 2B).

The solubility and CD spectra of these constructs may be dominated by the MBP fusion partner, but we did not attempt to remove it, as a shorter hydrophobic domain was reported to further aggregate under these circumstances (48).

The RTX domain comprises ˜40 RTX motifs, grouped into five blocks of calcium binding Gly-Asp repeats, separated by non-RTX flanking regions (FIG. 1A). Structural data for RTX-containing proteins suggest the repeats fold into parallel beta-helix structures. In the presence of calcium ions, the protein converts from an intrinsically disordered domain into a compact β-roll structure with altered CD spectrum and reduced hydrodynamic radius which appears to be further stabilized by acylation (43,49,50). RTX₉₈₅ exhibited a shift from largely monomer in the presence of calcium (78 kDa) to a mixture of oligomers (˜380 kDa) upon the addition of EGTA to chelate calcium ions. These structural changes are captured by CD, which shows a more ordered state in the presence of calcium ions (FIG. 2C), consistent with that observed with a similar construct also lacking the acylation sites (residues 1006-1706) (51). SDS-PAGE indicates the two peaks observed with calcium have the same molecular weight suggesting that RTX₉₈₅ forms two stable states with different hydrodynamic radii (FIG. 2C).

Theorizing that these two forms are a consequence of the missing acylation sites, we generated a larger construct to include both sites. RTX₇₅₁ expressed without CyaC eluted as a single peak of expected size (˜110 kDa) in the presence or absence of calcium. When co-expressed with CyaC, presumably resulting in acylation at residues K860 and K983, RTX₇₅₁* exhibited a calcium-dependent conversion from a soluble higher-molecular weight aggregate (˜600 kDa) to a compact monomer (˜90 kDa) in the presence of calcium (FIG. 2D).

The catalytic and RTX domains retain much of the expected structural behavior, with RTX₇₅₁* appearing to better stabilize the monomeric form than RTX₉₈₅. Similar to previous reports on ACT behavior (26,27,51-53), the presence of calcium and acylation appears to stabilize the RTX monomers. Anecdotally, the RTX domains were stable for at least six months at 4° C. with minimal aggregation or degradation (RTX₇₅₁ and RTX₇₅₁* were slightly more stable than RTX₉₈₅), as measured by SDS-PAGE and SEC to assess the monomeric fraction. While the CAT₄₀₀ domain also remains monomeric, it starts to degrade after three months under the same conditions, as measured by SDS-PAGE. The compact β-roll structure RTX domains adopt in the presence of calcium may contribute to their overall higher melting temperature and resistance to proteolysis than CAT (Table 1).

### Example 3: ACT Domains are Biochemically Similar to ACT

To determine if our domain constructs retain structural elements present in ACT, we screened a panel of nine previously characterized monoclonal antibodies for binding to ACT and individual domains by ELISA (47). All nine antibodies tested recognized only the expected domain and did not distinguish between acylated and non-acylated domains (Table 2), supporting the notion that they are properly folded. One exception is 2B12, whose epitope includes residues 888-1006, did not recognize HP₁₀₉₆. This may be due to incomplete folding of HP₁₀₉₆ or the binding site may require additional residues distal to residue 1006 not present in this construct.

TABLE 2
Biochemical analysis of ACT constructs

| | | 3D1 | 2A12 | 10A1 | 2B12 | 6E1 | 9D4 | 7C7 | 1H6 | 10A8 |
|---|---|---|---|---|---|---|---|---|---|---|
| ACT | | 373- | 399- | 624- | 888- | 1320- | 1156- | 1320- | 1590- | 1590- |
| domain | Epitope | 399 | 828 | 780 | 1006 | 1489 | 1489 | 1627 | 1706 | 1706 |

| | | | | | | | | | |
|---|---|---|---|---|---|---|---|---|---|
| CAT₄₀₀ | +++ | | | | | | | | |
| HP₁₀₉₆ | | +++ | + | | | | | | |
| HP₁₀₉₆* | | +++ | + | | | | | | |
| RTX₉₈₅ | | | | | ++ | ++ | ++ | +++ | +++ |
| RTX₇₅₁ | | | | + | ++ | ++ | + | +++ | +++ |
| RTX₇₅₁* | | | | + | ++ | ++ | + | +++ | +++ |
| ACT | +++ | +++ | ++ | ++ | +++ | +++ | + | ++ | +++ |

Binding of previously characterized anti-ACT murine antibodies (47) to ACT and various derivatives described in this work in ELISA assays. ACT constructs were coated on ELISA plates, the antibodies titrated and detected with anti-mouse IgG-HRP.

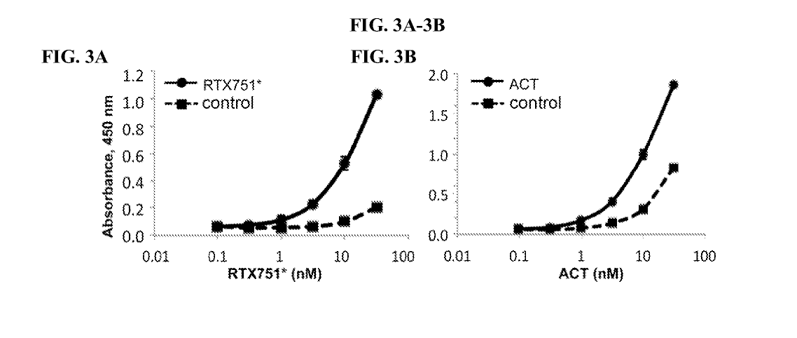

As the RTX domain harbors the receptor binding site between residues 1166-1281 (26), we assessed the ability of our RTX constructs to bind purified murine extracellular α_{M}β₂ receptor, a known ACT cell-surface receptor (25). While ACT and RTX₇₅₁* both bound the murine receptor when acylated and in the presence of calcium (apparent EC₅₀ ˜20 nM; FIG. 3), ACT exhibited considerable non-specific binding to wells without the receptor. This is similar to the sticky behavior observed when ACT without urea was applied to and retained by the SEC column and likely reflects solvent exposed hydrophobic patches in misfolded ACT molecules. The monomeric RTX₉₈₅ peak did not bind the α_{M}β₂ receptor, consistent with prior studies showing post-translational acylation is essential for receptor binding (29,54). In summary, the individual domains were readily purified, with yields of CAT₄₀₀ at ˜80 mg/L culture, non-acylated RTX and HP domains at ˜5 mg/L culture, and the acylated domains at <2 mg/L culture. The CAT and RTX domains share many structural features with ACT, while the HP domain is mostly aggregated.

### Example 4: Anti-ACT Antibodies Primarily Recognize RTX

To determine whether a single ACT domain dominates the immune response, we used phage display to analyze murine antibody repertoires after ACT immunization. Two mice were immunized intraperitoneally with 17 μg of ACT in complete Freund's adjuvant and boosted subcutaneously once with incomplete Freund's adjuvant. The resulting sera neutralized the toxic activities of ACT at a 1:400 dilution in an in vitro cAMP intoxication assay. The spleens were harvested, and each was used to construct an antibody phage-display library, each containing ˜10⁷ total transformants. After two rounds of panning against ACT, 90 individual clones from each library were grown in 96-well plates and assessed for ACT binding by phage ELISA. Of these, 57 and 60 clones from the two libraries respectively yielded signals two-fold above background, with 29 and 21 expressing unique sequences, as determined by BstNI fingerprinting and DNA sequencing.

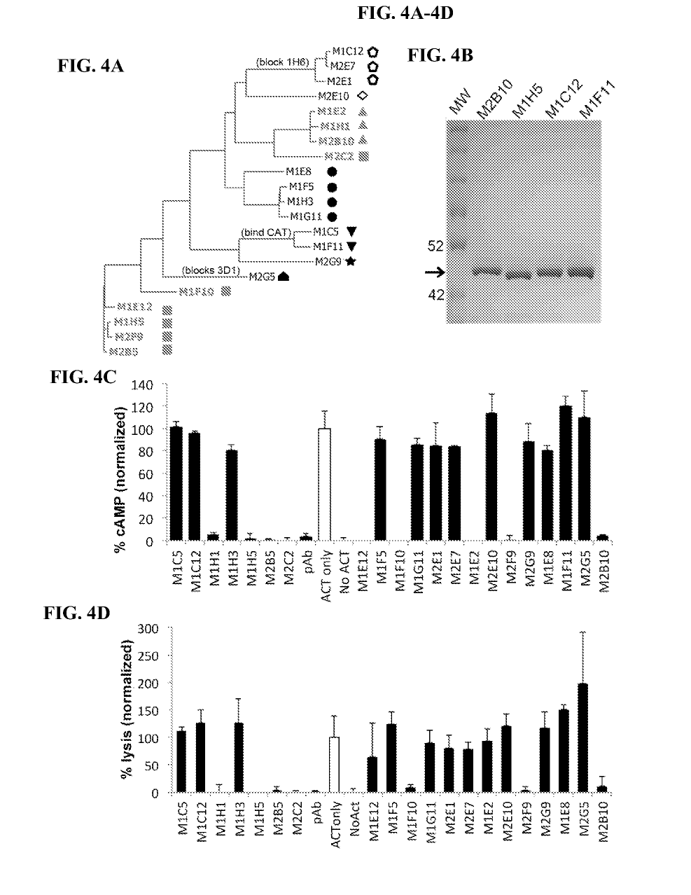

To determine the domain specificities of unique antibodies, monoclonal phage were assessed for binding to individual domains in ELISA (FIG. 4A). Few antibodies bound the catalytic domain; none bound the hydrophobic domain, while the rest (27 of 29 and 20 of 21, respectively) bound the RTX₉₈₅ domain.

### Example 5: Anti-RTX Antibodies can Neutralize ACT

Next, we screened unique antibodies identified from phage libraries for the ability to neutralize ACT activities using an in vitro cAMP assay. There are several steps during ACT intoxication of cells which are susceptible to antibody-mediated neutralization—including receptor binding, membrane insertion, and translocation—interference with any of these will be reflected in decreased intracellular cAMP accumulation in or reduced lysis of target cells. For this assay, we employed the murine macrophage cell line J774A.1 bearing the α_{M}β₂ integrin.

For the neutralization assay, antibodies were expressed as recombinant single-chain antibody fragments (scAb), comprised of the variable light chain (V_{L}) joined to a flexible (Gly₄Ser)₄ linker and the variable heavy chain (V_{H}), followed by a C-terminal human kappa chain constant region to increase solubility and serve as a detection handle (9). Based on multiple sequence alignment, 31 antibodies with unique CDR sequences were selected for scAb expression (FIG. 4B). Ten scAbs either expressed poorly (<200 μg/L culture) or bound ACT weakly (concentration >238 nM required for saturation) and were not tested further.

To identify antibodies neutralizing ACT function, ACT and individual scAbs were incubated at a 1:160 molar ratio before addition to adherent J774A.1 cells and determination of intracellular cAMP levels by competition ELISA. Of the 21 scAbs tested, nine reduced the cAMP level by more than 90%, as compared to cells treated with ACT alone, which we consider highly neutralizing in this assay (FIG. 4C). We also determined the ability of these scAbs to rescue J774A.1 cells from lysis using a lactate dehydrogenase release assay, observing a strong correlation with cAMP neutralization (FIG. 4D). This is in agreement with findings by Basler et al. that intracellular ATP depletion is sufficient to promote cell lysis (55). Notably, all neutralizing antibodies identified recognize the RTX domain.

### Example 6: Two Novel Neutralizing Epitopes in the RTX Domain

We next sought to classify neutralizing antibodies based on their recognition of unique or overlapping epitopes. Here, we used a competitive binding ELISA, in which a single phage-displayed antibody was mixed with buffer or a second antibody in the scAb format, added to an ELISA well coated with ACT, followed by detection of bound phage remaining in the well. Reduced signal in the presence of a second antibody compared to phage antibody alone indicates competition between the two antibodies for the same or overlapping epitopes. Using this approach, the nine neutralizing antibodies were divided into two groups binding non-overlapping epitopes, while non-neutralizing antibodies recognized four unique epitopes, for a total of six epitopes represented in this study (FIG. 4A).

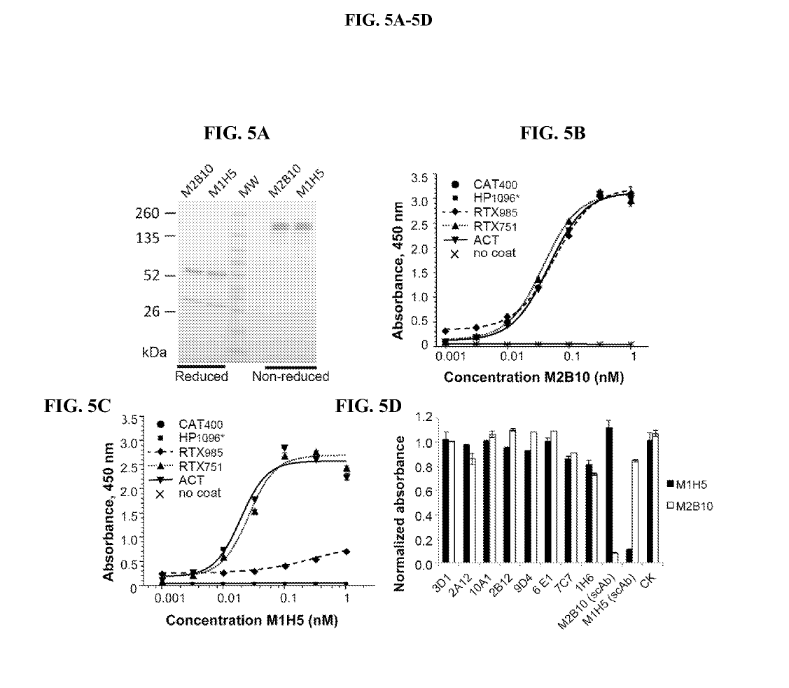

One representative antibody binding each neutralizing epitope was selected based on sequence uniqueness, expression level, and binding affinity for conversion into a full-length chimeric immunoglobulin, with human IgG1 and kappa constant domains (FIG. 5A). ELISA assays with the two RTX constructs helped to further define the epitopes recognized by these two antibodies, named M1H5 and M2B10. Both antibodies bound RTX₇₅₁* with almost the identical affinity as full-length ACT (FIG. 5B), while M1H5 bound the shorter RTX₉₈₅ domain weakly (FIG. 5C), suggesting that its epitope is not fully contained or properly presented in this construct.

To determine whether the antibodies identified here bind epitopes overlapping with those of previously defined murine monoclonal antibodies (47), we performed a second set of competition ELISAs (FIG. 5D). None of the murine antibodies competed with M2B10 or M1H5 for ACT binding, demonstrating that these antibodies recognize previously undescribed neutralizing epitopes in the RTX region.

Together, we observed antibodies binding six non-overlapping epitopes on RTX, two neutralizing and four non-neutralizing and all but two requiring the presence of calcium (FIG. 4A).

### Example 7: Antibodies Binding Novel RTX Neutralizing Epitopes Disrupt ACT-α_{M}β₂ Integrin Binding

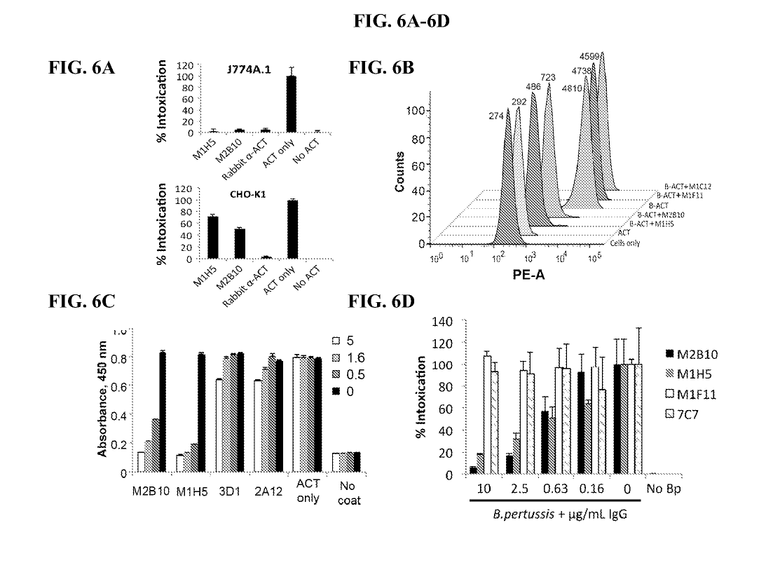

ACT primarily targets cells bearing the α_{M}β₂ receptor under conditions in which the RTX domain assumes a receptor-binding competent conformation mediated by the presence of calcium ions and post-translational acylation (26). Combining this with our observation that the M2B10 and M1H5 antibodies showed a much weaker neutralizing effect at the same 160-fold molar excess when CHO-K1 cells lacking this receptor were used than when J774A.1 cells expressing the receptor were used (FIG. 6A), we hypothesized that these two antibodies act by blocking the interaction between ACT and the α_{M}β₂ integrin.

To test this hypothesis, we used flow cytometry to monitor ACT bound to J774A.1 cells in the presence or absence of the M2B10 or M1H5 scAb. Biotinylated ACT was incubated with a 120-fold molar excess of neutralizing or non-neutralizing scAbs, added to J774A.1 cells and detected with phycoerythrin-conjugated streptavidin by FACS. The M2B10 and M1H5 antibodies significantly reduced binding of ACT-biotin to J774A.1 cells, while two non-neutralizing control scAbs (M1F11 and M1C12) had no significant effect (FIG. 6B). This difference was not due to affinity, as all four scAbs have similar affinities (EC₅₀=0.3-0.7 nM).

To further confirm that the diminished binding of ACT to J774A.1 cells was due to interference with a specific receptor, a competition ELISA with soluble α_{M}β₂ integrin was performed. ACT (0.5 μg/mL) was incubated with the M2B10, M1H5, M1F11 or 3D1 antibodies in molar ratios ranging from 5 to 0.5 and transferred to an α_{M}β₂ integrin-coated plate. The result was consistent with the flow cytometry assay: M2B10 and M1H5 reduced ACT binding to immobilized α_{M}β₂ integrin in a dose-dependent manner, >90% at a two-fold molar excess (FIG. 6C). In contrast, 3D1, a neutralizing IgG which blocks translocation of the catalytic domain (57) and 2A12, a neutralizing antibody with unclear mode-of-action, had no effect. Minimal non-specific binding was observed under these assay conditions.

To determine whether ACT neutralization occurs in the context of the whole bacterium, we repeated this assay with live B. pertussis instead of purified ACT. According to Gray et al., newly synthesized ACT is responsible for intoxication (28). Therefore, B. pertussis was washed in PBS to remove any secreted ACT. Bacteria (OD₆₀₀=0.2) added to J774A.1 cells resulted in cAMP levels similar to that induced by 125 ng/mL purified ACT. When the M2B10 and M1H5 but not the non-neutralizing M1F11 or 7C7 (58) antibodies were added with the bacteria, they resulted in dose-dependent reduction of cAMP levels (FIG. 6D). The result indicates that these antibodies can neutralize ACT in the context of active infection.

### Example 8: The RTX Domain is Immunodominant and Elicits Neutralizing Antibodies

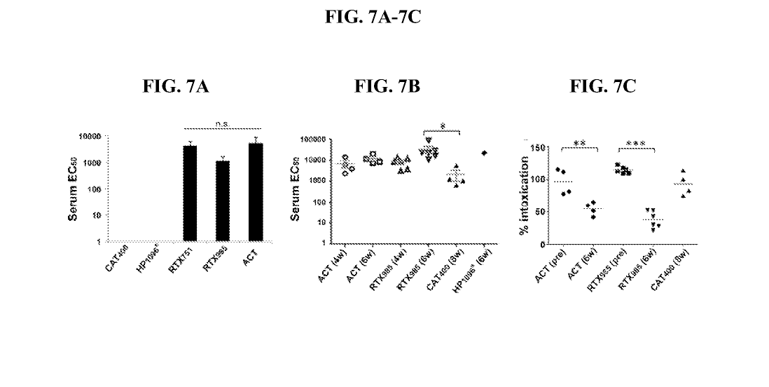

To determine if any ACT domain dominates the immune response, we immunized mice with ACT and tested the resulting sera four weeks after primary immunization for binding to different ACT domains. Interestingly, strong responses were observed for ACT, RTX₉₈₅ and RTX₇₅₁, but no responses were observed for the CAT₄₀₀ or HP₁₀₉₆* domains (FIG. 7A).

To determine whether this was the result of RTX immunodominance or a lack of immunogenicity by the CAT and HP domains, we immunized additional groups of mice with CAT₄₀, HP₁₀₉₆* or RTX₉₈₅. Here, RTX₉₈₅ was selected since it shares many features with RTX₇₅₁*, including recognition by at least one neutralizing antibody, yet lacks the acylation sites rendering it simpler to produce and less likely to engage the native receptor. The calcium concentration in extracellular fluid in the body is 2.2˜2.7 mM (59), which is sufficient to support bacterial secretion and folding of ACT during an infection and expected to support proper folding during immunization.

Mice immunized with ACT or RTX₉₈₅ showed high anti-ACT titers four weeks after the first injection, which increased after boosting (weeks 6 and 8, FIG. 7B). On the contrary, the catalytic domain was much less immunogenic, reaching a detectable anti-ACT titer of ˜1500 only after two boosts (week 8; FIG. 7B). This weak response may reflect structural similarities between CAT and eukaryotic adenylate cyclases, resulting in immunological tolerance or an evolutionary mechanism to protect key toxin components from neutralizing antibodies (60,61). Only one of the four mice immunized with the hydrophobic domain reacted with ACT, supporting the SEC data that this construct is poorly folded (FIG. 7B). Antibody responses in the three remaining mice were directed toward the MBP fusion, as determined by ELISA (data not shown).

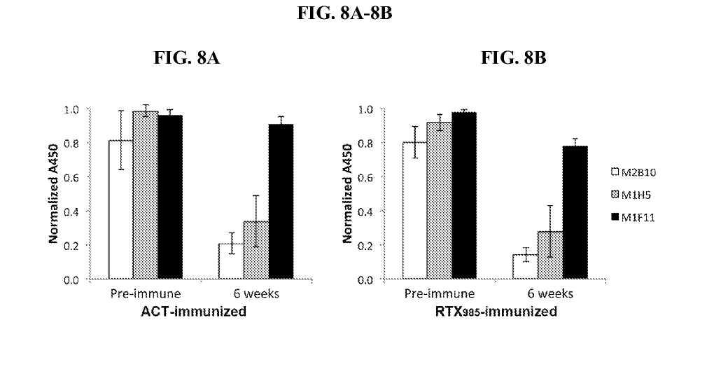

Next, we wanted to determine which domains induced sera best able to neutralize ACT cAMP intoxication activities in vitro with J774A.1 cells. Here, only immunization with RTX₉₈₅ elicited sera able to protect cells to a similar extent as sera elicited by full-length ACT (FIG. 7C). Although mechanisms other than receptor blockade may contribute to neutralization, the presence of M2B10- and M1H5-like antibodies in the sera of ACT- or RTX-immunized animals was confirmed by competition ELISA (FIG. 8). Since RTX₉₈₅ binds antibody M1H5 weakly, it is possible that this domain harbors critical residues and sufficient conformational similarities to induce overlapping but non-identical M1H5-like antibodies.

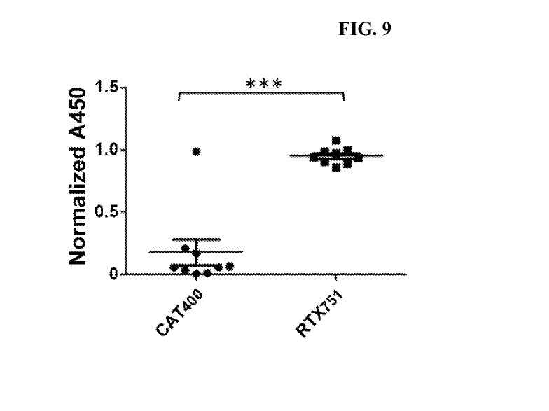

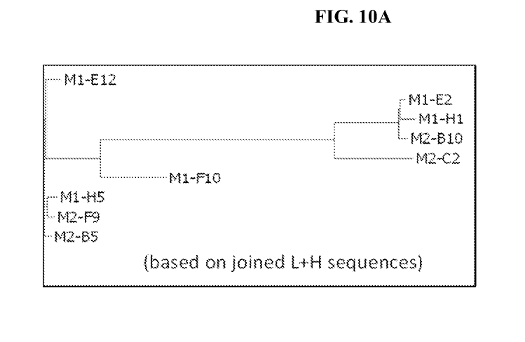

Finally, to determine whether humans show a similar bias towards RTX recognition, we tested nine serum samples from humans exposed to B. pertussis, selected randomly from a larger collection (62). All nine sera recognized RTX₇₅₁ at a similar level as full-length ACT, while sera from only one individual bound CAT (FIG. 9). Taken together, these data provide proof of-concept that RTX dominates the anti-ACT immune response and that the RTX domain can recapitulate the humoral immune responses induced by ACT.

C. Discussion

The recent surge in pertussis cases, coupled with increasing recognition of the current acellular vaccine's shortcomings has motivated design of third generation vaccines to prevent pertussis. In humans, even a single dose of whole cell vaccine significantly reduces the risk of illness (63), while in baboons, acellular immunization prevented the severe symptoms of disease, but allowed bacterial persistence and transmission to naïve animals (5). In order to design future vaccines which minimize sub-clinical disease and reduce transmission to susceptible infants, it is crucial to understand the roles played by various protective antigens. ACT has been shown to be protective in animal models and is immunogenic in humans (15,16,40,64,65). Since ACT activities hinder local anti-bacterial immune responses (66-68), anti-ACT antibodies may protect these cells, indirectly facilitating bacterial elimination. Here, we demonstrated that the RTX domain is able to largely recapitulate the protective humoral immune response induced by ACT in mice and is better expressed and more stable than the intact ACT.

ACT was first discovered based on its ability to increase cAMP levels in neutrophils, inhibiting their anti-bacterial functions, including phagocytosis and respiratory burst and promoting the early stages of disease establishment (66). At physiologically relevant concentrations (<50 ng/ml) (69), ACT results in cytotoxicity to the murine macrophage cell line J774A.1 after two hours exposure; IL-2 secretion and proliferation of T cells and chlorine efflux from polarized epithelial cells (42). More recently, ACT has been shown to suppress development of an IL-17 mediated immune response which appears key for bacterial clearance (21). As a result, passively administered antibodies blocking ACT function may be able to enhance neutrophil-mediated phagocytosis of opsonized bacteria (67). Murine studies have shown that immunization with ACT alone or as a supplement to the acellular vaccine reduces bacterial colonization, an effect which correlated with increased immunoglobulin levels and a Th1/Th2 cytokine phenotype (16,54). Finally, ACT is a highly conserved antigen, able to induce protective immunity in mouse models against the three dominant Bordetella species (B. pertussis, B. parapertussis, and B. bronchiseptica) (70-72). While ACT is unlikely to be highly protective as an isolated antigen, it may be a valuable addition to vaccines.

The complex mechanism by which ACT directly translocates its catalytic domain into the host cell cytosol remains incompletely understood. The current working model consists of three steps (48,73,74). First, in the presence of millimolar levels of calcium and acylation, the RTX domain forms a beta barrel which binds the α_{M}β₂ receptor on neutrophils or the β₂-containing integrin LFA receptor on T cells through N-linked oligosaccharides. Second, this is followed by insertion of two loops (four predicted transmembrane alpha helices between residues 502-522 and 565-591) into the host cell membrane, resulting in a translocation intermediate which permeabilizes the membrane to allow an influx of extracellular calcium ions, activating calpain-mediated cleavage of the integrin's talin tether. Third, the ACT-receptor complex is then free to diffuse to cholesterol-rich lipid rafts, which triggers complete translocation of the catalytic domain dependent on residues 375-485 (48). Interestingly, no specific sequence contained in the catalytic domain is required for translocation, allowing this region to be replaced with other sequences for intracellular delivery (75).

This structure-function information provides insight into epitopes required for cellular intoxication and those likely to induce protective responses. For instance, antibodies could block translocation steps, yet only one such antibody has been characterized. Immunization with ACT, followed by analysis of the resulting polyclonal serum suggested that antibodies recognizing the RTX domain dominate the response, as 6 of 12 monoclonal antibodies recognized this domain (47,72). Of these, the 3D1 antibody binds a conformational epitope between residues 373-399, adjacent to the catalytic domain, apparently trapping a translocation intermediate and preventing complete delivery of the catalytic domain to the host cytosol (48,73), while the anti-hydrophobic region antibody 2A12 inhibits intoxication and to a lesser extent, hemolysis, and the anti-RTX antibody 6E1 inhibits only hemolysis (47). Consistent with these prior reports, we observed that the majority of antibodies recovered from phage libraries bind the RTX domain, while sera from four mice immunized with the holo-toxin bind RTX only. To the best of our knowledge, no antibodies blocking ACT-receptor binding, such as M1H5 or M2B10, have been previously described.

A barrier to development of additional technologies based on ACT has been the challenges of recovering monomeric protein from in vitro refolding processes. Standard protocols, including dilution and dialysis from denaturing buffers containing 8 M urea recover high molecular weight species with variable activity levels. Recently, refolding on a size exclusion column was performed to prevent aggregation of partially folded species and resulted in purification of monomers with very high activity, dependent on the presence of calcium and acylation and molecular confinement (43). While promising, the yields and scalability of this process are currently unclear. As shown here, the RTX domain presents an alternative to ACT that retains many structural features, is more readily expressed and exhibits greater stability. Furthermore, since RTX lacks the catalytic domain, it has no homology to endogenous proteins and thus poses no potential autoimmunity concerns for human use. We evaluated two different RTX constructs, initially RTX₉₈₅ lacking the acylation sites and thus simpler to express and then the larger RTX₇₅₁* retaining the acylation sites. While both exhibited expected calcium dependent structural shifts and binding to previously described monoclonal antibodies, RTX₇₅₁* appears superior to RTX₉₈₅ in terms of monomericity, stability and recognition of soluble α_{M}β₂ receptor in vitro.

Since RTX₉₈₅ is well behaved and binds at least one neutralizing antibody (M2B10), we used this construct for immunization before discovering that RTX₇₅₁ and RTX₇₅₁* are better behaved and retain the ability to bind both neutralizing antibodies. Regardless, sera from mice immunized with RTX₉₈₅ neutralized cAMP intoxication in vitro as efficiently as sera from ACT-immunized mice (FIG. 7C). This may be because these non-overlapping epitopes induce antibodies neutralizing toxin via similar receptor blocking mechanisms. Supporting this idea, no synergy was observed when the M2B10 and M1H5 antibodies were combined in vitro. Thus, it may be possible to induce a strong neutralizing response when only one of the epitopes is structurally intact.

### Example 9: RTX Vaccines

RTX fragments are equally immunogenic as intact ACT and more immunogenic than the N-terminal catalytic and hydrophobic fragments (FIG. 11). In two separate mouse studies, the immunogenicities of ACT and RTX were evaluated. In the first experiment, mice were injected with antigens in complete Freund's adjuvant and 4 weeks later, they were boosted at two week intervals with incomplete Freund's adjuvant. In the second experiment, mice with injected with antigens in alum and 3 weeks later, they were boosted with antigens in alum again. One week later, sera were tested. The N-terminal catalytic domain (CAT, AA 1-400) and hydrophobic domain (HP, AA 384-1906) were less immunogenic than those including the RTX domain. Serum reactivities to full-length ACT and RTX fragments were indistinguishable. (4 w) refers to 4 weeks after the primary immunization, boosts were performed at 4 weeks & 6 weeks.

### VIII. References

1. Witt, M. A., Katz, P. H., and Witt, D. J. (2012) Unexpectedly limited durability of immunity following acellular pertussis vaccination in preadolescents in a North American outbreak. Clin Infect Dis 54, 1730-1735
2. Octavia, S., Sintchenko, V., Gilbert, G. L., Lawrence, A., Keil, A. D., Hogg, G., and Lan, R. (2012) Newly emerging clones of Bordetella pertussis carrying prn2 and ptxP3 alleles implicated in Australian pertussis epidemic in 2008-2010. J Infect Dis 205, 1220-1224
3. Higgs, R., Higgins, S. C., Ross, P. J., and Mills, K. H. (2012) Immunity to the respiratory pathogen Bordetella pertussis. Mucosal immunology 5, 485-500
4. Sheridan, S. L., Ware, R. S., Grimwood, K., and Lambert, S. B. (2012) Number and order of whole cell pertussis vaccines in infancy and disease protection. JAMA 308, 454-456
5. Warfel, J. M., Zimmerman, L. I., and Merkel, T. J. (2014) Acellular pertussis vaccines protect against disease but fail to prevent infection and transmission in a nonhuman primate model. Proc Natl Acad Sci USA 111, 787-792
6. Lim, A., Ng, J. K., Locht, C., and Alonso, S. (2014) Protective role of adenylate cyclase in the context of a live pertussis vaccine candidate. Microbes Infect 16, 51-60
7. Polewicz, M., Gracia, A., Garlapati, S., van Kessel, J., Strom, S., Halperin, S. A., Hancock, R. E., Potter, A. A., Babiuk, L. A., and Gerdts, V. (2013) Novel vaccine formulations against pertussis offer earlier onset of immunity and provide protection in the presence of maternal antibodies. Vaccine 31, 3148-3155
8. Cherry, J. D., Heininger, U., Richards, D. M., Storsaeter, J., Gustafsson, L., Ljungman, M., and Hallander, H. O. (2010) Antibody response patterns to Bordetella pertussis antigens in vaccinated (primed) and unvaccinated (unprimed) young children with pertussis. Clin Vaccine Immunol 17, 741-747
9. Berbers, G. A., de Greeff, S. C., and Mooi, F. R. (2009) Improving pertussis vaccination. Hum Vaccin 5, 497-503
10. Chenal-Francisque, V., Caro, V., Boursaux-Eude, C., and Guiso, N. (2009) Genomic analysis of the adenylate cyclase-hemolysin C-terminal region of Bordetella pertussis, Bordetella parapertussis and Bordetella bronchiseptica. Res Microbiol 160, 330-336
11. Parkhill, J., Sebaihia, M., Preston, A., Murphy, L. D., Thomson, N., Harris, D. E., Holden, M. T. G., Churcher, C. M., Bentley, S. D., Mungall, K. L., Cerdeno-Tarraga, A. M., Temple, L., James, K., Harris, B., Quail, M. A., Achtman, M., Atkin, R., Baker, S., Basham, D., Bason, N., Cherevach, I., Chillingworth, T., Collins, M., Cronin, A., Davis, P., Doggett, J., Feltwell, T., Goble, A., Hamlin, N., Hauser, H., Holroyd, S., Jagels, K., Leather, S., Moule, S., Norberczak, H., O'Neil, S., Ormond, D., Price, C., Rabbinowitsch, E., Rutter, S., Sanders, M., Saunders, D., Seeger, K., Sharp, S., Simmonds, M., Skelton, J., Squares, R., Squares, S., Stevens, K., Unwin, L., Whitehead, S., Barrell, B. G., and Maskell, D. J. (2003) Comparative analysis of the genome sequences of Bordetella pertussis, Bordetella parapertussis and Bordetella bronchiseptica. Nat Genet 35, 32-40
12. Carbonetti, N. H., Artamonova, G. V., Andreasen, C., and Bushar, N. (2005) Pertussis toxin and adenylate cyclase toxin provide a one-two punch for establishment of Bordetella pertussis infection of the respiratory tract. Infection and immunity 73, 2698
13. Goodwin, M., and Weiss, A. (1990) Adenylate cyclase toxin is critical for colonization and pertussis toxin is critical for lethal infection by Bordetella pertussis in infant mice. Infection and immunity 58, 3445
14. Khelef, N., Sakamoto, H., and Guiso, N. (1992) Both adenylate cyclase and hemolytic activities are required by Bordetella pertussis to initiate infection. Microbial pathogenesis 12, 227-235
15. Guiso, N., Rocancourt, M., Szatanik, M., and Alonso, J.-M. (1989) Bordetella adenylate cyclase is a virulence associated factor and an immunoprotective antigen. Microbial pathogenesis 7, 373-380
16. Guiso, N., Szatanik, M., and Rocancourt, M. (1991) Protective activity of Bordetella adenylate cyclase-hemolysin against bacterial colonization. Microbial pathogenesis 11, 423-431
17. Farfel, Z., Konen, S., Wiertz, E., Klapmuts, R., Addy, P. A. K., and Hanski, E. (1990) Antibodies to Bordetella pertussis adenylate cyclase are produced in man during pertussis infection and after vaccination. Journal of medical microbiology 32, 173-177
18. Guo, Q., Shen, Y., Lee, Y. S., Gibbs, C. S., Mrksich, M., and Tang, W. J. (2005) Structural basis for the interaction of Bordetella pertussis adenylyl cyclase toxin with calmodulin. EMBO J 24, 3190-3201
19. Raptis, A., Knipling, L., and Wolff, J. (1989) Dissociation of catalytic and invasive activities of Bordetella pertussis adenylate cyclase. Infect Immun 57, 1725-1730
20. Paccani, S. R., Dal Molin, F., Benagiano, M., Ladant, D., D'Elios, M. M., Montecucco, C., and Baldari, C. T. (2008) Suppression of T-lymphocyte activation and chemotaxis by the adenylate cyclase toxin of Bordetella pertussis. Infect Immun 76, 2822-2832
21. Henderson, M. W., Inatsuka, C. S., Sheets, A. J., Williams, C. L., Benaron, D. J., Donato, G. M., Gray, M. C., Hewlett, E. L., and Cotter, P. A. (2012) Contribution of Bordetella Filamentous Hemagglutinin and Adenylate Cyclase Toxin to Suppression and Evasion of Interleukin-17-Mediated Inflammation. Infection and immunity 80, 2061-2075
22. Rossi Paccani, S., Benagiano, M., Capitani, N., Zornetta, I., Ladant, D., Montecucco, C., D'Elios, M. M., and Baldari, C. T. (2009) The adenylate cyclase toxins of Bacillus anthracis and Bordetella pertussis promote Th2 cell development by shaping T cell antigen receptor signaling. PLoS Pathog 5, e1000325
23. Benz, R., Maier, E., Ladant, D., Ullmann, A., and Sebo, P. (1994) Adenylate cyclase toxin (CyaA) of Bordetella pertussis. Evidence for the formation of small ion-permeable channels and comparison with HlyA of Escherichia coli. J Biol Chem 269, 27231-27239
24. Hackett, M., Walker, C. B., Guo, L., Gray, M. C., Van Cuyk, S., Ullmann, A., Shabanowitz, J., Hunt, D. F., Hewlett, E. L., and Sebo, P. (1995) Hemolytic, but not cell-invasive activity, of adenylate cyclase toxin is selectively affected by differential fatty-acylation in Escherichia coli. Journal of Biological Chemistry 270, 20250-20253
25. Guermonprez, P., Khelef, N., Blouin, E., Rieu, P., Ricciardi-Castagnoli, P., Guiso, N., Ladant, D., and Leclerc, C. (2001) The adenylate cyclase toxin of Bordetella pertussis binds to target cells via the α_{M}β2 integrin (CD11b/CD18). The Journal of experimental medicine 193, 1035
26. El-Azami-El-Idrissi, M., Bauche, C., Loucka, J., Osicka, R., Sebo, P., Ladant, D., and Leclerc, C. (2003) Interaction of Bordetella pertussis adenylate cyclase with CD11b/CD18: Role of toxin acylation and identification of the main integrin interaction domain. J Biol Chem 278, 38514-38521
27. Basar, T., Havlicek, V., Bezouskova, S., Hackett, M., and Sebo, P. (2001) Acylation of lysine 983 is sufficient for toxin activity of Bordetella pertussis adenylate cyclase. Substitutions of alanine 140 modulate acylation site selectivity of the toxin acyltransferase CyaC. J Biol Chem 276, 348-354
28. Gray, M. C., Donato, G. M., Jones, F. R., Kim, T., and Hewlett, E. L. (2004) Newly secreted adenylate cyclase toxin is responsible for intoxication of target cells by Bordetella pertussis. Mol Microbiol 53, 1709-1719
29. Betsou, F., Sebo, P., and Guiso, N. (1993) CyaC-mediated activation is important not only for toxic but also for protective activities of Bordetella pertussis adenylate cyclase-hemolysin. Infection and immunity 61, 3583
30. Wilkins, M. R., Gasteiger, E., Bairoch, A., Sanchez, J. C., Williams, K. L., Appel, R. D., and Hochstrasser, D. F. (1999) Protein identification and analysis tools in the ExPASy server. Methods Mol Biol 112, 531-552
31. Whitmore, L., and Wallace, B. A. (2004) DICHROWEB, an online server for protein secondary structure analyses from circular dichroism spectroscopic data. Nucleic Acids Res 32, W668-673
32. Krebber, A., Bornhauser, S., Burmester, J., Honegger, A., Willuda, J., Bosshard, H. R., and Pluckthun, A. (1997) Reliable cloning of functional antibody variable domains from hybridomas and spleen cell repertoires employing a reengineered phage display system. J Immunol Methods 201, 35-55
33. Hayhurst, A., Happe, S., Mabry, R., Koch, Z., Iverson, B. L., and Georgiou, G. (2003) Isolation and expression of recombinant antibody fragments to the biological warfare pathogen Brucella melitensis. J Immunol Methods 276, 185-196
34. Maynard, J., Adams, E. J., Krogsgaard, M., Petersson, K., Liu, C. W., and Garcia, K. C. (2005) High-level bacterial secretion of single-chain alphabeta T-cell receptors. J Immunol Methods 306, 51-67
35. Smith, K., Garman, L., Wrammert, J., Zheng, N. Y., Capra, J. D., Ahmed, R., and Wilson, P. C. (2009) Rapid generation of fully human monoclonal antibodies specific to a vaccinating antigen. Nat Protoc 4, 372-384
36. Karimova, G., and Ladant, D. (2007) cAMP assays. CSH Protoc 2007, pdb prot4739
37. Glaser, P., Ladant, D., Sezer, O., Pichot, F., Ullmann, A., and Danchin, A. (1988) The calmodulin-sensitive adenylate cyclase of Bordetella pertussis: cloning and expression in Escherichia coli. Mol Microbiol 2, 19-30
38. Hewlett, E. L., Gordon, V. M., McCaffery, J. D., Sutherland, W. M., and Gray, M. C. (1989) Adenylate cyclase toxin from Bordetella pertussis. Identification and purification of the holotoxin molecule. J Biol Chem 264, 19379-19384
39. Rogel, A., Schultz, J. E., Brownlie, R. M., Coote, J. G., Parton, R., and Hanski, E. (1989) Bordetella pertussis adenylate cyclase: purification and characterization of the toxic form of the enzyme. EMBO J 8, 2755-2760
40. Brézin, C., Guiso, N., Ladant, D., Djavadi-Ohaniance, L., Megret, F., Onyeocha, I., and Alonso, J.-M. (1987) Protective effects of anti-Bordetella pertussis adenylate cyclase antibodies against lethal respiratory infection of the mouse. FEMS Microbiology Letters 42, 75-80
41. Rogel, A., Farfel, Z., Goldschmidt, S., Shiloach, J., and Hanski, E. (1988) Bordetella pertussis adenylate cyclase. Identification of multiple forms of the enzyme by antibodies. J Biol Chem 263, 13310-13316
42. Eby, J. C., Gray, M. C., Mangan, A. R., Donato, G. M., and Hewlett, E. L. (2012) Role of CD11b/CD18 in the process of intoxication by the adenylate cyclase toxin of Bordetella pertussis. Infect Immun 80, 850-859
43. Karst, J. C., Ntsogo Enguene, V. Y., Cannella, S. E., Subrini, O., Hessel, A., Debard, S., Ladant, D., and Chenal, A. (2014) Calcium, acylation and molecular confinement favor folding of Bordetella pertussis adenylate cyclase CyaA toxin into a monomeric and cytotoxic form. J Biol Chem 289:30702-16
44. Gallay, J., Vincent, M., Li de la Sierra, I. M., Munier-Lehmann, H., Renouard, M., Sakamoto, H., Bârzu, O., and Gilles, A. M. (2004) Insight into the activation mechanism of Bordetella pertussis adenylate cyclase by calmodulin using fluorescence spectroscopy. European Journal of Biochemistry 271, 821-833
45. Powthongchin, B., and Angsuthanasombat, C. (2008) High level of soluble expression in Escherichia coli and characterisation of the CyaA pore-forming fragment from a Bordetella pertussis Thai clinical isolate. Arch Microbiol 189, 169-174
46. Pojanapotha, P., Thamwiriyasati, N., Powthongchin, B., Katzenmeier, G., and Angsuthanasombat, C. (2011) Bordetella pertussis CyaA-RTX subdomain requires calcium ions for structural stability against proteolytic degradation. Protein Expr Purif 75, 127-132
47. Lee, S. J., Gray, M., Guo, L., Sebo, P., and Hewlett, E. (1999) Epitope mapping of monoclonal antibodies against Bordetella pertussis adenylate cyclase toxin. Infection and immunity 67, 2090
48. Karst, J. C., Barker, R., Devi, U., Swann, M. J., Davi, M., Roser, S. J., Ladant, D., and Chenal, A. (2012) Identification of a region that assists membrane insertion and translocation of the catalytic domain of Bordetella pertussis CyaA toxin. J Biol Chem 287, 9200-9212
49. Blenner, M. A., Shur, O., Szilvay, G. R., Cropek, D. M., and Banta, S. (2010) Calcium-induced folding of a beta roll motif requires C-terminal entropic stabilization. J Mol Biol 400, 244-256
50. Sotomayor Perez, A. C., Karst, J. C., Davi, M., Guijarro, J. I., Ladant, D., and Chenal, A. (2010) Characterization of the regions involved in the calcium-induced folding of the intrinsically disordered RTX motifs from the bordetella pertussis adenylate cyclase toxin. JMol Biol 397, 534-549
51. Bauche, C., Chenal, A., Knapp, O., Bodenreider, C., Benz, R., Chaffotte, A., and Ladant, D. (2006) Structural and functional characterization of an essential RTX subdomain of Bordetella pertussis adenylate cyclase toxin. J Biol Chem 281, 16914-16926
52. Chenal, A., Guijarro, J. I., Raynal, B., Delepierre, M., and Ladant, D. (2009) RTX calcium binding motifs are intrinsically disordered in the absence of calcium: implication for protein secretion. J Biol Chem 284, 1781-1789
53. Masin, J., Basler, M., Knapp, O., El-Azami-El-Idrissi, M., Maier, E., Konopasek, I., Benz, R., Leclerc, C., and Sebo, P. (2005) Acylation of lysine 860 allows tight binding and cytotoxicity of Bordetella adenylate cyclase on CD11b-expressing cells. Biochemistry 44, 12759-12766
54. Cheung, G. Y. C., Xing, D., Prior, S., Corbel, M. J., Parton, R., and Coote, J. G. (2006) Effect of different forms of adenylate cyclase toxin of Bordetella pertussis on protection afforded by an acellular pertussis vaccine in a murine model. Infection and immunity 74, 6797
55. Basler, M., Masin, J., Osicka, R., and Sebo, P. (2006) Pore-forming and enzymatic activities of Bordetella pertussis adenylate cyclase toxin synergize in promoting lysis of monocytes. Infect Immun 74, 2207-2214
56. Saggy, I., Wine, Y., Shefet-Carasso, L., Nahary, L., Georgiou, G., and Benhar, I. (2012) Antibody isolation from immunized animals: comparison of phage display and antibody discovery via V gene repertoire mining. Protein Eng Des Sel 25, 539-549
57. Gray, M. C., Lee, S. J., Gray, L. S., Zaretzky, F. R., Otero, A. S., Szabo, G., and Hewlett, E. L. (2001) Translocation-specific conformation of adenylate cyclase toxin from Bordetella pertussis inhibits toxin-mediated hemolysis. J Bacteriol 183, 5904-5910
58. Lee, S. J., Gray, M. C., Guo, L., Sebo, P., and Hewlett, E. L. (1999) Epitope mapping of monoclonal antibodies against Bordetella pertussis adenylate cyclase toxin. Infect Immun 67, 2090-2095
59. Goldstein, D. A. (1990) Serum Calcium. Clinical Methods: The History, Physical, and Laboratory Examinations. 3rd edition. Boston: Butterworths
60. Goyard, S., Orlando, C., Sabatier, J. M., Labruyere, E., d'Alayer, J., Fontan, G., van Rietschoten, J., Mock, M., Danchin, A., Ullmann, A., and et al. (1989) Identification of a common domain in calmodulin-activated eukaryotic and bacterial adenylate cyclases. Biochemistry 28, 1964-1967
61. Monneron, A., Ladant, D., d'Alayer, J., Bellalou, J., Barzu, O., and Ullmann, A. (1988) Immunological relatedness between Bordetella pertussis and rat brain adenylyl cyclases. Biochemistry 27, 536-539
62. Sutherland, J. N., Chang, C., Yoder, S. M., Rock, M. T., and Maynard, J. A. (2011) Antibodies recognizing protective pertussis toxin epitopes are preferentially elicited by natural infection versus acellular immunization. Clin Vaccine Immunol 18, 954-962
63. Witt, M. A., Arias, L., Katz, P. H., Truong, E. T., and Witt, D. J. (2013) Reduced risk of pertussis among persons ever vaccinated with whole cell pertussis vaccine compared to recipients of acellular pertussis vaccines in a large US cohort. Clin Infect Dis 56, 1248-1254
64. Watanabe, M., Connelly, B., and Weiss, A. A. (2006) Characterization of serological responses to pertussis. Clin Vaccine Immunol 13, 341-348
65. Hormozi, K., Parton, R., and Coote, J. (1999) Adjuvant and protective properties of native and recombinant Bordetella pertussis adenylate cyclase toxin preparations in mice. FEMS Immunol Med Microbiol 23, 273-282
66. Confer, D. L., and Eaton, J. W. (1982) Phagocyte impotence caused by an invasive bacterial adenylate cyclase. Science 217, 948-950
67. Weingart, C. L., Mobberley-Schuman, P. S., Hewlett, E. L., Gray, M. C., and Weiss, A. A. (2000) Neutralizing antibodies to adenylate cyclase toxin promote phagocytosis of Bordetella pertussis by human neutrophils. Infection and immunity 68, 7152
68. Weingart, C. L., and Weiss, A. A. (2000) Bordetella pertussis virulence factors affect phagocytosis by human neutrophils. Infection and immunity 68, 1735
69. Eby, J. C., Gray, M. C., Warfel, J. M., Paddock, C. D., Jones, T. F., Day, S. R., Bowden, J., Poulter, M. D., Donato, G. M., Merkel, T. J., and Hewlett, E. L. (2013) Quantification of the adenylate cyclase toxin of Bordetella pertussis in vitro and during respiratory infection. Infect Immun 81, 1390-1398
70. Gueirard, P., and Guiso, N. (1993) Virulence of Bordetella bronchiseptica: role of adenylate cyclase-hemolysin. Infect Immun 61, 4072-4078
71. Khelef, N., Danve, B., Quentin-Millet, M. J., and Guiso, N. (1993) Bordetella pertussis and Bordetella parapertussis: two immunologically distinct species. Infect Immun 61, 486-490
72. Betsou, F., Sebo, P., and Guiso, N. (1995) The C-terminal domain is essential for protective activity of the Bordetella pertussis adenylate cyclase-hemolysin. Infection and immunity 63, 3309
73. Bumba, L., Masin, J., Fiser, R., and Sebo, P. (2010) Bordetella adenylate cyclase toxin mobilizes its beta2 integrin receptor into lipid rafts to accomplish translocation across target cell membrane in two steps. PLoS Pathog 6, e1000901
74. Basler, M., Knapp, O., Masin, J., Fiser, R., Maier, E., Benz, R., Sebo, P., and Osicka, R. (2007) Segments crucial for membrane translocation and pore-forming activity of Bordetella adenylate cyclase toxin. Journal of Biological Chemistry 282, 12419
75. Preville, X., Ladant, D., Timmerman, B., and Leclerc, C. (2005) Eradication of established tumors by vaccination with recombinant Bordetella pertussis adenylate cyclase carrying the human papillomavirus 16 E7 oncoprotein. Cancer Res 65, 641-649
76. Scarselli, M., Arico, B., Brunelli, B., Savino, S., Di Marcello, F., Palumbo, E., Veggi, D., Ciucchi, L., Cartocci, E., Bottomley, M. J., Malito, E., Lo Surdo, P., Comanducci, M., Giuliani, M. M., Cantini, F., Dragonetti, S., Colaprico, A., Doro, F., Giannetti, P., Pallaoro, M., Brogioni, B., Tontini, M., Hilleringmann, M., Nardi-Dei, V., Banci, L., Pizza, M., and Rappuoli, R. (2011) Rational design of a meningococcal antigen inducing broad protective immunity. Sci Transl Med 3, 91ra62
77. McLellan, J. S., Chen, M., Joyce, M. G., Sastry, M., Stewart-Jones, G. B., Yang, Y., Zhang, B., Chen, L., Srivatsan, S., Zheng, A., Zhou, T., Graepel, K. W., Kumar, A., Moin, S., Boyington, J. C., Chuang, G. Y., Soto, C., Baxa, U., Bakker, A. Q., Spits, H., Beaumont, T., Zheng, Z., Xia, N., Ko, S. Y., Todd, J. P., Rao, S., Graham, B. S., and Kwong, P. D. (2013) Structure-based design of a fusion glycoprotein vaccine for respiratory syncytial virus. Science 342, 592-598
78. Angkawidjaja, C., You, D. J., Matsumura, H., Kuwahara, K., Koga, Y., Takano, K., and Kanaya, S. (2007) Crystal structure of a family 1.3 lipase from Pseudomonas sp. MIS38 in a closed conformation. FEBS Lett 581, 5060-5064
79. Lupardus, P. J., Shen, A., Bogyo, M., and Garcia, K. C. (2008) Small molecule-induced allosteric activation of the Vibrio cholerae RTX cysteine protease domain. Science 322, 265-268

### IX. P Embodiments

### Embodiment P1

An antibody that competes for binding to the repeat in toxin (RTX) domain of Bordetella adenylate cyclase toxin (ACT) with an antibody comprising sequences selected from the group consisting of: light chain CDR1, CDR2, and CDR3 sequences of SEQ ID NO:1 and heavy chain CDR1, CDR2, and CDR3 sequences of SEQ ID NO:2; light chain CDR1, CDR2, and CDR3 sequences of SEQ ID NO:3 and heavy chain CDR1, CDR2, and CDR3 sequences of SEQ ID NO:4; light chain CDR1, CDR2, and CDR3 sequences of SEQ ID NO:5 and heavy chain CDR1, CDR2, and CDR3 sequences of SEQ ID NO:6; light chain CDR1, CDR2, and CDR3 sequences of SEQ ID NO:7 and heavy chain CDR1, CDR2, and CDR3 sequences of SEQ ID NO:9; light chain CDR1, CDR2, and CDR3 sequences of SEQ ID NO:9 and heavy chain CDR1, CDR2, and CDR3 sequences of SEQ ID NO:10; and light chain CDR1, CDR2, and CDR3 sequences of SEQ ID NO:11 and heavy chain CDR1, CDR2, and CDR3 sequences of SEQ ID NO:12.

### Embodiment P2

The antibody of embodiment P1, wherein the antibody comprises sequences selected from the group consisting of: light chain CDR1, CDR2, and CDR3 sequences of SEQ ID NO:1 and heavy chain CDR1, CDR2, and CDR3 sequences of SEQ ID NO:2; light chain CDR1, CDR2, and CDR3 sequences of SEQ ID NO:3 and heavy chain CDR1, CDR2, and CDR3 sequences of SEQ ID NO:4; light chain CDR1, CDR2, and CDR3 sequences of SEQ ID NO:5 and heavy chain CDR1, CDR2, and CDR3 sequences of SEQ ID NO:6; light chain CDR1, CDR2, and CDR3 sequences of SEQ ID NO:7 and heavy chain CDR1, CDR2, and CDR3 sequences of SEQ ID NO:9; light chain CDR1, CDR2, and CDR3 sequences of SEQ ID NO:9 and heavy chain CDR1, CDR2, and CDR3 sequences of SEQ ID NO:10; and light chain CDR1, CDR2, and CDR3 sequences of SEQ ID NO:11 and heavy chain CDR1, CDR2, and CDR3 sequences of SEQ ID NO:12.

### Embodiment P3

The antibody of embodiment P1 or P2, wherein the antibody comprises sequences selected from the group consisting of: light chain variable region sequence of SEQ ID NO:1 and heavy chain variable region sequence of SEQ ID NO:2; light chain variable region sequence of SEQ ID NO:3 and heavy chain variable region sequence of SEQ ID NO:4; light chain variable region sequence of SEQ ID NO:5 and heavy chain variable region sequence of SEQ ID NO:6; light chain variable region sequence of SEQ ID NO:7 and heavy chain variable region sequence of SEQ ID NO:9; light chain variable region sequence of SEQ ID NO:9 and heavy chain variable region sequence of SEQ ID NO:10; and light chain variable region sequence of SEQ ID NO:11 and heavy chain variable region sequence of SEQ ID NO:12.

### Embodiment P4

An antibody that competes for binding to the repeat in toxin (RTX) domain of Bordetella adenylate cyclase toxin (ACT) with an antibody comprising sequences selected from the group consisting of: light chain CDR1, CDR2, and CDR3 sequences of SEQ ID NO:13 and heavy chain CDR1, CDR2, and CDR3 sequences of SEQ ID NO:14; light chain CDR1, CDR2, and CDR3 sequences of SEQ ID NO:15 and heavy chain CDR1, CDR2, and CDR3 sequences of SEQ ID NO:16; and light chain CDR1, CDR2, and CDR3 sequences of SEQ ID NO:17 and heavy chain CDR1, CDR2, and CDR3 sequences of SEQ ID NO:18.

### Embodiment P5

The antibody of embodiment P4, wherein the antibody comprises sequences selected from the group consisting of: light chain CDR1, CDR2, and CDR3 sequences of SEQ ID NO:13 and heavy chain CDR1, CDR2, and CDR3 sequences of SEQ ID NO:14; light chain CDR1, CDR2, and CDR3 sequences of SEQ ID NO:15 and heavy chain CDR1, CDR2, and CDR3 sequences of SEQ ID NO:16; and light chain CDR1, CDR2, and CDR3 sequences of SEQ ID NO:17 and heavy chain CDR1, CDR2, and CDR3 sequences of SEQ ID NO:18.

### Embodiment P6

The antibody of embodiment P4 or P5, wherein the antibody comprises sequences selected from the group consisting of: light chain variable region sequence of SEQ ID NO:13 and heavy chain variable region sequence of SEQ ID NO:14; light chain variable region sequence of SEQ ID NO:15 and heavy chain variable region sequence of SEQ ID NO:16; and light chain variable region sequence of SEQ ID NO:17 and heavy chain variable region sequence of SEQ ID NO:18.

### Embodiment P7

The antibody of any one of the foregoing embodiments, wherein the antibody is humanized.

### Embodiment P8

The antibody of any one of the foregoing embodiments, wherein the antibody is a single chain antibody (scAb).

### Embodiment P9

The antibody of embodiment P8, wherein the scAb comprises a light chain variable region, a linker consisting of 4-40 amino acids, and a heavy chain variable region.

### Embodiment P10

The antibody of embodiment P8 or P9, wherein the scAb further comprises a constant region.

### Embodiment P11

The antibody of any one of embodiments P8-P10, wherein the linker consists of 16-24 amino acids.

### Embodiment P12

The antibody of any one of the foregoing embodiments, further comprising a detectable label.

### Embodiment P13

A pharmaceutical composition comprising the antibody of any one of the foregoing embodiments and a pharmaceutically acceptable excipient.

### Embodiment P14

The pharmaceutical composition of embodiment P13, comprising at least one additional antibody specific for the RTX domain.

### Embodiment P15

A method of treating an individual exposed to Bordetella bacteria comprising administering the pharmaceutical composition of any one of embodiments P13-P14 to the individual, thereby treating the individual.

### X. Embodiments

### Embodiment 1

A vaccine composition comprising a recombinant Bordetella adenylate cyclase toxin polypeptide consisting essentially of a sequence corresponding to amino acid residues 700-1706 of Bordetella adenylate cyclase toxin protein or an immunogenic fragment thereof and a pharmaceutically acceptable vaccine excipient.

### Embodiment 2

The vaccine composition of embodiment 1, wherein said recombinant Bordetella adenylate cyclase toxin polypeptide consists of a sequence corresponding to amino acid residues 700-1706 of Bordetella adenylate cyclase toxin protein or an immunogenic fragment thereof and a pharmaceutically acceptable vaccine excipient.

### Embodiment 3

The vaccine composition of embodiment 1 or 2, wherein said sequence corresponds to amino acid residues 751-1706 of Bordetella adenylate cyclase toxin protein or a fragment thereof.

### Embodiment 4

The vaccine composition of one of embodiments 1-3, wherein said sequence corresponds to amino acid residues 985-1706 of Bordetella adenylate cyclase toxin protein or a fragment thereof.

### Embodiment 5

The vaccine composition of one of embodiments 1-4, wherein said sequence corresponds to amino acid residues 1000-1706 of Bordetella adenylate cyclase toxin protein or a fragment thereof.

### Embodiment 6

The vaccine composition of one of embodiments 1-5, wherein said sequence is SEQ ID NO:31, SEQ ID NO:32, SEQ ID NO:33, SEQ ID NO:34, SEQ ID NO:35, SEQ ID NO:36, SEQ ID NO:37 or SEQ ID NO:38.

### Embodiment 7

The vaccine composition of one of embodiments 1-6, wherein said sequence comprises an acylated amino acid residue.

### Embodiment 8

The vaccine composition of embodiment 7, wherein said sequence comprises an acylated amino acid residue at a position corresponding to K983 or K860.

### Embodiment 9

The vaccine composition of embodiment 7, wherein said acylated residue is a lysine.

### Embodiment 10

The vaccine composition of one of embodiments 1-9, wherein said Bordetella adenylate cyclase toxin polypeptide binds α_{M}β₂ integrin.

### Embodiment 11

The vaccine composition of one of embodiments 1-6, wherein said sequence is a non-acylated sequence.

### Embodiment 12

The vaccine composition of one of embodiments 1-6 or 11, wherein said sequence comprises a non-acylated amino acid residue at a position corresponding to K983 or K860.

### Embodiment 13

The vaccine composition of embodiment 11, wherein said non-acylated residue is a lysine.

### Embodiment 14

The vaccine composition of one of embodiments 11-13, wherein said Bordetella adenylate cyclase toxin polypeptide does not bind α_{M}β₂ integrin.

### Embodiment 15

The vaccine composition of one of embodiments 1-14, further comprising an adjuvant.

### Embodiment 16

A method for immunizing a subject in need thereof against Bordetella pertussis, the method comprising administering to said subject an effective amount of the vaccine of one of embodiments 1-15.

### Embodiment 17

A method of preventing or treating whooping cough in a subject in need thereof, the method comprising administering to said subject a therapeutically effective amount of the vaccine of one of embodiments 1-15.

1. A vaccine composition comprising a recombinant Bordetella adenylate cyclase toxin polypeptide consisting essentially of a sequence corresponding to amino acid residues 700-1706 of Bordetella adenylate cyclase toxin protein or an immunogenic fragment thereof and a pharmaceutically acceptable vaccine excipient.

2. The vaccine composition of claim 1, wherein said recombinant Bordetella adenylate cyclase toxin polypeptide consists of a sequence corresponding to amino acid residues 700-1706 of Bordetella adenylate cyclase toxin protein or an immunogenic fragment thereof and a pharmaceutically acceptable vaccine excipient.

3. The vaccine composition of claim 1, wherein said sequence corresponds to amino acid residues 751-1706 of Bordetella adenylate cyclase toxin protein or a fragment thereof.

4. The vaccine composition of claim 1, wherein said sequence corresponds to amino acid residues 985-1706 of Bordetella adenylate cyclase toxin protein or a fragment thereof.

5. The vaccine composition of claim 1, wherein said sequence corresponds to amino acid residues 1000-1706 of Bordetella adenylate cyclase toxin protein or a fragment thereof.

6. The vaccine composition of claim 1, wherein said sequence is SEQ ID NO:31, SEQ ID NO:32, SEQ ID NO:33, SEQ ID NO:34, SEQ ID NO:35, SEQ ID NO:36, SEQ ID NO:37 or SEQ ID NO:38.

7. The vaccine composition of claim 1, wherein said sequence comprises an acylated amino acid residue.

8. The vaccine composition of claim 7, wherein said sequence comprises an acylated amino acid residue at a position corresponding to K983 or K860.

9. The vaccine composition of claim 7, wherein said acylated residue is a lysine.

10. The vaccine composition of claim 1, wherein said Bordetella adenylate cyclase toxin polypeptide binds αMβ2 integrin.

11. The vaccine composition of claim 1, wherein said sequence is a non-acylated sequence.

12. The vaccine composition of claim 1, wherein said sequence comprises a non-acylated amino acid residue at a position corresponding to K983 or K860.

13. (canceled)

14. The vaccine composition of claim 11, wherein said Bordetella adenylate cyclase toxin polypeptide does not bind αMβ2 integrin.

15. The vaccine composition of claim 1, further comprising an adjuvant.

16. A method for immunizing a subject in need thereof against Bordetella pertussis, the method comprising administering to said subject an effective amount of the vaccine of claim 1.

17. A method of preventing or treating whooping cough in a subject in need thereof, the method comprising administering to said subject a therapeutically effective amount of the vaccine of claim 1.

18. An antibody that competes for binding to the repeat in toxin (RTX) domain of Bordetella adenylate cylase toxin (ACT) with an antibody comprising sequences selected from the group consisting of:
light chain CDR1, CDR2, and CDR3 sequences of SEQ ID NO: 1 and heavy chain CDR1, CDR2, and CDR3 sequences of SEQ ID NO:2;
light chain CDR1, CDR2, and CDR3 sequences of SEQ ID NO:3 and heavy chain CDR1, CDR2, and CDR3 sequences of SEQ ID NO:4;
light chain CDR1, CDR2, and CDR3 sequences of SEQ ID NO:5 and heavy chain CDR1, CDR2, and CDR3 sequences of SEQ ID NO:6;
light chain CDR1, CDR2, and CDR3 sequences of SEQ ID NO:7 and heavy chain CDR1, CDR2, and CDR3 sequences of SEQ ID NO:9;
light chain CDR1, CDR2, and CDR3 sequences of SEQ ID NO:9 and heavy chain CDR1, CDR2, and CDR3 sequences of SEQ ID NO: 10; and
light chain CDR1, CDR2, and CDR3 sequences of SEQ ID NO: 11 and heavy chain CDR1, CDR2, and CDR3 sequences of SEQ ID NO: 12.

19. An antibody that competes for binding to the repeat in toxin (RTX) domain of Bordetella adenylate cylase toxin (ACT) with an antibody comprising sequences selected from the group consisting of:
light chain CDR1, CDR2, and CDR3 sequences of SEQ ID NO: 13 and heavy chain CDR1, CDR2, and CDR3 sequences of SEQ ID NO:14;
light chain CDR1, CDR2, and CDR3 sequences of SEQ ID NO:15 and heavy chain CDR1, CDR2, and CDR3 sequences of SEQ ID NO:16; and
light chain CDR1, CDR2, and CDR3 sequences of SEQ ID NO: 17 and heavy chain CDR1, CDR2, and CDR3 sequences of SEQ ID NO:18.

20. A pharmaceutical composition comprising the antibody of claim 18 and a pharmaceutically acceptable excipient.

21. A method of treating an individual exposed to Bordetella bacteria comprising administering the pharmaceutical composition of claim 20 to the individual, thereby treating the individual.


SEQUENCE LISTING (38 sequences)

SEQ ID NO: 1 - protein, 110 aa, Artificial Sequence
  Synthetic polypeptide
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRGG

SEQ ID NO: 2 - protein, 121 aa, Artificial Sequence
  Synthetic polypeptide
EVNLVESGGDLVKPGGSLKLSCAASGFTFSSYGMSWVRQTPDKRLEWVATISSGGTYTYYPDSVKGRFTISRDNAKNTLYLQMSSLKSEDTAMYYCAREIMRGGGYYFDYWSQGTTLTVSS

SEQ ID NO: 3 - protein, 111 aa, Artificial Sequence
  Synthetic polypeptide
DIVLTQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIKRGG

SEQ ID NO: 4 - protein, 118 aa, Artificial Sequence
  Synthetic polypeptide
QVQLQQSGPELGKPGASVKISCKASGYLFTGYNIYWVKQSHRKSLEWIGYINPYNGDTSYNQKSKGKATLTVDRSSSTAYMHLNSLTSEDSAIYFCARGYYVRPLDYWGQGTTLTVSS

SEQ ID NO: 5 - protein, 114 aa, Artificial Sequence
  Synthetic polypeptide
DIVMTQSPASLAVSLGQRATISCRASESVGNYGISFTNWYQQKPGQPPKLLIYAASNLESGIPARFSGSGSGTDFTLNIHPVEEEDAATYYCQQSNEDPYTFGGGTKLEIKRGG

SEQ ID NO: 6 - protein, 121 aa, Artificial Sequence
  Synthetic polypeptide
EVKLVESGGDLVKPGGSLKLSCAASGFTFSSYGMSWVRQTPDKRLEWVATISSGGTYTYYPDSVKGRFTISRDNAKNTLYLQMSSLKSEDTAMYYCAREIMRGGGYYFDYWGQGTTLTVSS

SEQ ID NO: 7 - protein, 110 aa, Artificial Sequence
  Synthetic polypeptide
DIVMTQATSSLSASLGDRVTISCRASQDISNYLNWYQQKPDGTVKLLIYYTSRSHSGVPSRFSGSGSGTDYSLTISNLEQEDIATYSCQQGNTLPYTFGGGTKLEIKRGG

SEQ ID NO: 8 - protein, 121 aa, Artificial Sequence
  Synthetic polypeptide
  misc_feature (114)..(115): Xaa can be any naturally occurring amino acid
EVKLMESGGDLVKPGGSLKLSCAASGFTFSSYGMSWVRQTQDKRLEWVATISSGGTYTYYPDSVKGRFTISRDNAKNTLYLQMSSLKSEDTAMYYCAREIMRGGGYYFDYWGQXXTLTVSS

SEQ ID NO: 9 - protein, 109 aa, Artificial Sequence
  Synthetic polypeptide
DIQMTQSTSSLSASLGDRVTISCRASQDISNYLNWYQQKPDGTVKLLIYYTSRLHSGVPSRFSGRGSGTDYSLTISNLEQEDIATYFCQQGNTLYTFGGGTKLEIKRGG

SEQ ID NO: 10 - protein, 121 aa, Artificial Sequence
  Synthetic polypeptide
  misc_feature (89)..(89): Xaa can be any naturally occurring amino acid
EVKLVESGGDLVKPGGSLKLSCAASGFTFSSYGMSWVRQTPDKRLEWVATISSGGTYTYYPDSVKGRFTISRDNAKNTLYLQMSSLKSXDTAMYYCAREIMRGGDYYFDYWGQGTTLTVSS

SEQ ID NO: 11 - protein, 110 aa, Artificial Sequence
  Synthetic polypeptide
DIVMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELKRGG

SEQ ID NO: 12 - protein, 121 aa, Artificial Sequence
  Synthetic polypeptide
  misc_feature (113)..(113): Xaa can be any naturally occurring amino acid
EVKLVESGGDLVKPGGSLKLSCAASGFTFSSYGMSWVRQTPDKRLEWVATISSGGTYTYYPDSVKGRFTISRDNAKNTLYLQMSSLKSEDTAMYYCAREIMRGGGYYFDYWGXGTTLTVSS

SEQ ID NO: 13 - protein, 111 aa, Artificial Sequence
  Synthetic polypeptide
DIVMTQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLEIKRGG

SEQ ID NO: 14 - protein, 123 aa, Artificial Sequence
  Synthetic polypeptide
QVQLKQSGAELVRPGTSVKVSCKASGYAFTNYLIEWVKQRPGQGLEWIGVINPGIGNTNYNEKFKGKATLTADKSSSTVYMQLSSLTSDDSAVYFCARGLNYGSSQHWYFDVWGAGTTVTVSS

SEQ ID NO: 15 - protein, 111 aa, Artificial Sequence
  Synthetic polypeptide
DIVMTQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLELKRGG

SEQ ID NO: 16 - protein, 123 aa, Artificial Sequence
  Synthetic polypeptide
QVQLQQSGAELVRPGTSVKVSCKASGYAFTNYLIEWVKQRPGQGLEWIGVINPGIGNTNYNEKFKGKATLTADKFSSTASMQLSSLTSDDSAVYFCARGLNYGSSQHWYFDVWGAGTSVTVSS

SEQ ID NO: 17 - protein, 111 aa, Artificial Sequence
  Synthetic polypeptide
DIVMTQSPAILSASLGERVTMTCTASSSVSSSYLHWYQQKPGSSPKLWIYSTSNLASGVPARFSGSGSGTSYSLTISSMEAEDAATYYCHQYHRSPPTFGAGTKLEIKRGG

SEQ ID NO: 18 - protein, 123 aa, Artificial Sequence
  Synthetic polypeptide
EVQLQQSGAELVRPGTSVKVSCKASGYAFTNYLIEWVKQRPGQGLEWIGVINPGIGNTNYNEKFKGKATLTADKSSSTVYMQLSSLTSDDSAVYFCARGLNYGSSQHWYFDVWGAGTSVTVSS

SEQ ID NO: 19 - DNA, 34 bp, Artificial Sequence
  Synthetic polynucleotide
aggaaacacatatgcagcaatcgcatcaggctgg

SEQ ID NO: 20 - DNA, 32 bp, Artificial Sequence
  Synthetic polynucleotide
actagaattcttacgaacgtccgctcggcacg

SEQ ID NO: 21 - DNA, 37 bp, Artificial Sequence
  Synthetic polynucleotide
cacagaattcttacgccggcaccgtttccagtacatc

SEQ ID NO: 22 - DNA, 31 bp, Artificial Sequence
  Synthetic polynucleotide
catagaattcttactggcgttccactgcgcc

SEQ ID NO: 23 - DNA, 26 bp, Artificial Sequence
  Synthetic polynucleotide
tcacgaacatatggccaattcggacg

SEQ ID NO: 24 - DNA, 27 bp, Artificial Sequence
  Synthetic polynucleotide
ctacggccatatgacggagaatgtcca

SEQ ID NO: 25 - DNA, 26 bp, Artificial Sequence
  Synthetic polynucleotide
ataggatcctcagcgccagttgacag

SEQ ID NO: 26 - DNA, 29 bp, Artificial Sequence
  Synthetic polynucleotide
gggcgcacatatgcgccaggattccggct

SEQ ID NO: 27 - DNA, 60 bp, Artificial Sequence
  Synthetic polynucleotide
atcggcggatccttaatggtgatgatggtgatgggcgctggcctcggaaggctggtgcac

SEQ ID NO: 28 - protein, 1706 aa, Bordetella pertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 29 - protein, 1706 aa, Bordetella bronchiseptica
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 30 - protein, 1706 aa, Bordetella parapertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAVGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 31 - protein, 956 aa, Bordetella pertussis
ANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 32 - protein, 977 aa, Artificial Sequence
  Synthetic polypeptide
MGSSHHHHHHSSGLVPRGSHMANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 33 - protein, 722 aa, Bordetella pertussis
TENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 34 - protein, 743 aa, Artificial Sequence
  Synthetic polypeptide
MGSSHHHHHHSSGLVPRGSHMTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 35 - protein, 956 aa, Bordetella bronchiseptica
ANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 36 - protein, 979 aa, Artificial Sequence
  Synthetic polypeptide
MGSSHHHHHHSSGLVPRGSHMASANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 37 - protein, 956 aa, Bordetellaparapertussis
ANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 38 - protein, 979 aa, Artificial Sequence
  Synthetic polypeptide
MGSSHHHHHHSSGLVPRGSHMASANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR